SEQUENCE LISTING (54 sequences)

SEQ ID NO: 1 - DNA, 1374 bp, Quillaja saponaria
  misc_feature: A nucleic acid molecule comprising a nucleotide which encodes the enzyme according to SEQ ID NO 2.
atggagaatgggagagtttacaaatcccatgtcgtggtgctcgcatttcacgggcaaggccatattgttccgttaatccaattatccagacgattggcctggaaaggcatcaaaatcacatttgctacaacacattcttgcaccaaggccattcaaacaggaagtgattcaatttcacttttatcaatttatgatgacataactgatggtgggtttcaaggagaaggaggattcaagggcttccttcagagatttgaagccagtaccacaaggatcttacacgaattcgtcaagaatcatgaaaactcaaagaacccagtaaaatgcttaatatatgatgctaacttaatatgggctctggaaatggcaaagcaattgggtattgctactgctgcatttgtgtttccttcttgggctgccattgccacctactatcccttttatttagaggtgtatgcggatcagcagataaagaaggtagatcctttcacaatgcctgacttacctccacaacttggacttccaaatatggcatctctcggttcagattcgggtcaacactcccccatactcaaactcatgttgcaacagttagaaaattttgggaaagctgactggatcctgtctcacgcatttgaacagtttgaacaagaggtacttgactggatgagaaatatcagcccagtaacaacaattggtccaactctgccatctgtttatcttgatggtaggctaaaagatgacacagattacggttacaatttgtacaagccagatagtgatacctgcatgaagtggctagacactaaggaaactgaatcagtggtttatatatcatttggcagtgttgcagatttgatcccagaacagatgacagaaataacaaactccctgaagaaaatgagcagcaactttctgtgggtggtgaaggaaactgaaaaaaacaacctccctagcagctttgttgaggagacaaaagaaaagggattggtagtgacttggtgcccccagttgaaggtgttgtctcatcctgcagtgggttgtttcattacacactgtggaacaaattccatatttgagtcagtatgctttgcagtgccaatggtgggaatgccacagttttgtgatcaaatgcctaatgcatatttcatggagaaggtctggaaagtaggtgttaggccaagtttggatgacaatggtgtcgtcactggagaagaaattgagcgatgtataaaagtagttaccgaaggagagagtgggcaagagattaagaagaaacttgtgcagtggaaagagcttgcaaaagaggcagtggacgagggtggaagttcagataagcacattgatgaattcattgctggaatcacaacttga

SEQ ID NO: 2 - protein, 457 aa, Quillaja saponaria
  misc_feature: A fucosyltransferase enzyme capable of transferring beta-d-fucopyranose to the C-28 position of Quillaic acid (Qs-28-O-FucT).
MENGRVYKSHVVVLAFHGQGHIVPLIQLSRRLAWKGIKITFATTHSCTKAIQTGSDSISLLSIYDDITDGGFQGEGGFKGFLQRFEASTTRILHEFVKNHENSKNPVKCLIYDANLIWALEMAKQLGIATAAFVFPSWAAIATYYPFYLEVYADQQIKKVDPFTMPDLPPQLGLPNMASLGSDSGQHSPILKLMLQQLENFGKADWILSHAFEQFEQEVLDWMRNISPVTTIGPTLPSVYLDGRLKDDTDYGYNLYKPDSDTCMKWLDTKETESVVYISFGSVADLIPEQMTEITNSLKKMSSNFLWVVKETEKNNLPSSFVEETKEKGLVVTWCPQLKVLSHPAVGCFITHCGTNSIFESVCFAVPMVGMPQFCDQMPNAYFMEKVWKVGVRPSLDDNGVVTGEEIERCIKVVTEGESGQEIKKKLVQWKELAKEAVDEGGSSDKHIDEFIAGITT

SEQ ID NO: 3 - DNA, 1422 bp, Quillaja saponaria
  misc_feature: A nucleic acid molecule comprising a nucleotide which encodes the enzyme according to SEQ ID NO 4.
atggcaaaaactgataagcagcttcacatcgccatgttcccatggctagctatgggtcatatattcccaaactttgagctcgctaagctctttgctcaaaagggtcactcaattactttaatctccaccccacgaaacatcagtcgtctccctcaaatccctacacatttagagcaattgattaaattagtcagcttgcctatattacccaaacacaaagcaaatctcccagagaatgcagagtccaccatggacgttacccccaataaagtcccataccttaagatggcctatgatggtcttcaggagtcgttgactcaattactgaaatcttcagctcccgattggattctatatgactttgctgctgactggttaccaccacttgttcacagccttcaaatccgctgtgttttcttcgtggtatcccctgcgtggaatctttgcttctttgacactcccaaaccacagttgggcagtgctgctgtttttcgaacaaagcctgaagactatcttcgccctcccagttgggttcctttccattcaaatattgggctaaagcttcacgaggtgaagaaaatgtttgaaggggtttcagataaagaaacaggggtcactgtaagttttaacttcaacaaagcagtttcgagctgtgacttgttttctttccgcagctgctatgaactcgaatcagaatggctgaacctggtggaggatatttacaagaggcctgtagttccagtgggcgtaattccaccctcttttcaagtcagaattgtgaatgaagaagacaacaaaccagagtggttaaagatccaatcttggttagataaacaagagcaaggatcggtggtatacatagcatttggcagtgagcttaagctgggccaacaagatctcaccgaattagctcttggacttgagctttctgggttgccattcttttgggcacttagaaagcagcaagacagctcatcagtagatttaccagatgggtttgaggaccgagtcagtgatcgtggagttgtttgcagagactgggtgccccaacttaagatcctagctcacgggtcaattgggggttatttgactcactgtggttcaggttcagtgatagagggacttcattttgggcgtgttcttgttatgctgccctatttactagaccaagcattatatgctagagtattggaggagaaaaagctgggggttgagataccaaggaacgaacaagatgggtcttttactaggagctcagtggccaagtctgtgaagttggccatagtggatgaggggggaagtatttacagggacaaagccaaagagatgggcttggtattcagtgacaaagatcgtcatgaacaatacattgagaatttccttcaacaccttcaacacaaaagggaacctttccaaatttaa

SEQ ID NO: 4 - protein, 473 aa, Quillaja saponaria
  misc_feature: A rhamnosyltransferase enzyme, capable of transferring alpha-1,2-l-rhamnopyranose to QA-F (Qs-28-O-RhaT).
MAKTDKQLHIAMFPWLAMGHIFPNFELAKLFAQKGHSITLISTPRNISRLPQIPTHLEQLIKLVSLPILPKHKANLPENAESTMDVTPNKVPYLKMAYDGLQESLTQLLKSSAPDWILYDFAADWLPPLVHSLQIRCVFFVVSPAWNLCFFDTPKPQLGSAAVFRTKPEDYLRPPSWVPFHSNIGLKLHEVKKMFEGVSDKETGVTVSFNFNKAVSSCDLFSFRSCYELESEWLNLVEDIYKRPVVPVGVIPPSFQVRIVNEEDNKPEWLKIQSWLDKQEQGSVVYIAFGSELKLGQQDLTELALGLELSGLPFFWALRKQQDSSSVDLPDGFEDRVSDRGVVCRDWVPQLKILAHGSIGGYLTHCGSGSVIEGLHFGRVLVMLPYLLDQALYARVLEEKKLGVEIPRNEQDGSFTRSSVAKSVKLAIVDEGGSIYRDKAKEMGLVFSDKDRHEQYIENFLQHLQHKREPFQI

SEQ ID NO: 5 - DNA, 1395 bp, Quillaja saponaria
  misc_feature: A nucleic acid molecule comprising a nucleotide which encodes the enzyme according to SEQ ID NO 6.
atggctgctgcagctcccaatcacaggctccacatagcattcttcccatggttagcatttggtcacataaaccctttctttgagcttgcgaagctcattgctcaaaagggtcatcatatttctttcatttccaccccaagaaacatccaacgcctttcacaagttcctccacaattagcagattccatagatctagtgagcttaccagtaatccataattcaaacctcccagaaaacgcagagtccaccatggacattccacctgataaaaccccttaccttgggatgcttcacgacagtctcaaagaaccccttactcaattccttcaaactcattcccctgattggattctgtatgacttttcagctggttggcttgctgccatagtagaagaccttggtatctcccacggctacttttctatcattccctgttggaacataggcttcaatggacgccaaatgaatggtttccaaaagccagatatttcattgccctccgccgtgtcgcttaagaaatatgaggtgaagaaaatcatggatttggtcaaatcttttcccaaaattttggatgagtcggccactaaatccatagcttcgcattcgacctgtgaagtaatttttatacgaaattgccccgagattgaagcagattggttcgactatacttcgaaaattttcgataaaccagtggttccggtgggcgtagtgcccccatctgtgcacataactaacaaagagaaggacgagcatttcaacaaatggttggagatcaaagaatggttggatcaacaagacagaggttctgtaatttatatagcttttggaactgaatcactgccaaatcaggatgaaatcaccatgcttgctcaagggcttgagctatgtgggcttcctttcttttgggcattaaggaagtctaatgtggcttctgatcagccaaattcagactcagttgagctaccggagggatttgaagaacaaaccaaaggtcgtggaattgtgtggacgagttgggcacctcaacagagaattctgggtcacaattcaattgggggttttgtgactcactgtggttggagttcagtaatagaaggaattcactatggacggccactgattatgttccctctaacagtcgaacagtctctgaatgctaggattttgggggagaagaagttgggtatggaagtacccagagaagatgatgggtcttttacaggtgaagttgtggcagagacattgaagctggtattgctggaccaagatgggaaagtttacagggacaaggtaacagagatgagtaaggtatttggggacaaagacaaacatgagaaatacatgggtgatctacttgaattcttcaaaaattacaggtctcttaagaggaattag

SEQ ID NO: 6 - protein, 464 aa, Quillaja saponaria
  misc_feature: A xylosyltransferase enzyme capable of transferring beta-1,4-d-xylopyranose to QA-FR (Qs-28-O-XylT3)
MAAAAPNHRLHIAFFPWLAFGHINPFFELAKLIAQKGHHISFISTPRNIQRLSQVPPQLADSIDLVSLPVIHNSNLPENAESTMDIPPDKTPYLGMLHDSLKEPLTQFLQTHSPDWILYDFSAGWLAAIVEDLGISHGYFSIIPCWNIGFNGRQMNGFQKPDISLPSAVSLKKYEVKKIMDLVKSFPKILDESATKSIASHSTCEVIFIRNCPEIEADWFDYTSKIFDKPVVPVGVVPPSVHITNKEKDEHFNKWLEIKEWLDQQDRGSVIYIAFGTESLPNQDEITMLAQGLELCGLPFFWALRKSNVASDQPNSDSVELPEGFEEQTKGRGIVWTSWAPQQRILGHNSIGGFVTHCGWSSVIEGIHYGRPLIMFPLTVEQSLNARILGEKKLGMEVPREDDGSFTGEVVAETLKLVLLDQDGKVYRDKVTEMSKVFGDKDKHEKYMGDLLEFFKNYRSLKRN

SEQ ID NO: 7 - DNA, 1530 bp, Quillaja saponaria
  misc_feature: A nucleic acid molecule comprising a nucleotide which encodes the enzyme according to SEQ ID NO 8.
atggactccacccacttgcagccggccactactcctctgaaaattcacttcatacctttcatatctccgggtcacatcatcccactctctgaattggctcgtatctttgcttcacgcggtgagcatgtgactatcattaccactcctttcaacgctgacctacttcagaaatccattgacgaagacagagattccggcgagcacattggcatccacaccgttgaatggtcagccacagagctaggccttcctcatgggattgaaaatctcagcaacgtcactgacttggagaccgccattaagctacacagggccctcatgctaatgcagaaacagatggaagatttcatgacccgaaatccacccgattgtataattgccgacacgttttacccgtgggcctccgaatttgctaatcggatgggtatcccgagactcattttctacccgtggagtactttcgcgctctgtttgatggaatctattcgatctcccgactccccgcaccgaagattgagttcggattcggatccatttgtggttccgggtcttcctcacccgatcatcttgacccgttctcagcttccggaacacgatcgaaaggacatagcggacccggctgcccaactcatggatcagcataaagaaactgagatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagagagggtattcaagagtgatgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttgtttgggtggttcgcggaaaagataaacaaatcgatgttaatgacgatggggagaagacatggttgcctaaagggtttgaggaaagaatgaaaacagaaaataaaggtttgattgtaaggggatgggccccacaggtgctggttttggatcatccatcattgggatgtttcttgacgcattgcggctggaattctacgattgagggaatcacagcaggcgttcctttgatcacgtggccagtattcgccgagcaattctataatgagaagctaatcacgcaggtgcatgggaatggggtggtggttggttcagaggagtggatcatgttgttcaccgtcgctaaaagcttggtaagtagagacaaaattgagaatgctgtgaggaagataatggacggtggtgatgaggctgtacaaatcagaaggcgggcccgggaacttggagaaaaagcttggaaagctgcttcaactggggggtcctcctacaataatctaaccgcagcaattgaagaccttaagcggttgagagaggaccgttcgaagctgaaaacaaaaacaatttga

SEQ ID NO: 8 - protein, 509 aa, Quillaja saponaria
  misc_feature: A xylosyltransferase enzyme capable of transferring beta-1,3-d-xylopyranose to QA-FRX (Qs-28-O-XylT4)
MDSTHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 9 - DNA, 1530 bp, Quillaja saponaria
  misc_feature: A nucleic acid molecule comprising a nucleotide which encodes the enzyme according to SEQ ID NO 10.
atggactccacccacttgcagccggccactactcctctgaaaattcacgtcatacctttcatagctccgggtcacattatcccactctctgaattggctcgtatctttgcttcacgcggtgagcatgtgactatcataaccactcctttcaacgctgacctacttcagaaatccattgacgaagacagagattccggcgagcacattggcatccacaccgttgaatggtcagccacagagctaggccttcctcatggggttgaaaatctcagcaacgtcactgacttggagaccggcattaagctacacagggccctcgtgctaatgcagaaacagatggaagatttcatgacccgaaatccacccgattgtataattgccgacacgttttacccgtgggcctccgaatttgctaatcggatgggtatcccgagactcattttcttcccggggtgtactttcgcgctctgtttgatggaatctattcgatctcccgactccccgcaccgaagattgagttcggattcggacccatttgtggttccgggtcttcctcacccgatcatcttgacccgttctcaacttccggaacacgatcgagaggacatagcggacccggctgcccaattcatggatcagtgtaaagaagctgcgatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagaggggcttttcaaaagtgacgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttatttgggtggttcgcggcaaagataaacaaatcgatgttaatgacgatgaggagaagacatggttgcctaaagggtttgaggaaagaatgaaaacagaaaataaaggtttgattgtaaggggatgggccccacaggtgctggttttggatcatccatcgttgggatgtttcttgacgcattgcggctggaattctacgattgagggaatcacagcaggtgttcctttgatcacgtggccagtatactccgagcaattctataatgagaagctaatcacgcaggtgcatggtaatggggtgggggttggttcagaggagtggatcatgctgttcagcgtcgctaaaagtttggtaagtagagacaaaattgagaatgctgtgaggaagataatggacggtggtgatgaggctttagaaatcagaaggcgggcccgggaacttggagaaaaagctaggaaagctgcttcaattggggggtcctccgacaataatctaaccgcagcaattgaagaccttaagcggttgagagaggaccgttcgaagctgaaaacaaaaacaatttga

SEQ ID NO: 10 - protein, 509 aa, Quillaja saponaria
  misc_feature: An apiosyltransferase enzyme capable of transferring beta-1,3-d-apiofuranose to QA-FRX (Qs-28-O-ApiT4).
MDSTHLQPATTPLKIHVIPFIAPGHIIPLSELARIFASRGEHVTIITTPFNADLLQKSIDEDRDSGEHIGIHTVEWSATELGLPHGVENLSNVTDLETGIKLHRALVLMQKQMEDFMTRNPPDCIIADTFYPWASEFANRMGIPRLIFFPGCTFALCLMESIRSPDSPHRRLSSDSDPFVVPGLPHPIILTRSQLPEHDREDIADPAAQFMDQCKEAAMKSYGIILNNFAEIETEYTEHYKKITGHKVWHIGPAAAIVHRNAKEKAEGLFKSDEHDNNLVINWLNSKEPNSVVYVCFGSGCQFPDKQLYEIACGLELSGHQFIWVVRGKDKQIDVNDDEEKTWLPKGFEERMKTENKGLIVRGWAPQVLVLDHPSLGCFLTHCGWNSTIEGITAGVPLITWPVYSEQFYNEKLITQVHGNGVGVGSEEWIMLFSVAKSLVSRDKIENAVRKIMDGGDEALEIRRRARELGEKARKAASIGGSSDNNLTAAIEDLKRLREDRSKLKTKTI

SEQ ID NO: 11 - DNA, 897 bp, Quillaja saponaria
  misc_feature: A nucleic acid molecule comprising a nucleotide which encodes the enzyme according to SEQ ID NO 12.
atggcagaagcaacgcagaggtatgctgttgtgacaggatctaataagggaattggatttgggatatgcaagcagttggcttctaagggaatcaaagtagtgttaacagctagagatgagaagagaggtcttgaagcagttgagaaattgaaagaaattagtctggctggtcatgtggtttttcatcaactcgatgtgtctgatcctgctagtgttactagccttgaagatttcatcaaaacccagtttgggaagctagatattctggtaaacaatgctgggataacaggaacaactgtagatgctgatgctttagcagcttcaggcttcggtacagggggtgaacgtaagcctattgattggagtaagttagtgatacagacttatgaatcagttgaaaaagctttcaacacaaactattacggtggcaaaagaatgacagaagcacttatacccctcctccagctatcagactcacccaggattgttaatgtttcctctgctatgggacagttagagaatatacctagtggatgggcaaaggaagtgctcacagatgttgataacctaacagaaggaaaattggatgaggtttcaacccagtttttgaaagatttcaaagagggttcattggaaaccaaaggctggcctagtcttatgtcttcttatatagtctcaaaagctgttttaaatgcctacacaaggattcttgctaagaaatacccagctttctgcatcaattgtgtagatcctggctatgtgaagacagacataaaccatcatactggccaattaagtgttgatgaaggtgctgaaagtcctgtaagactggccttgctgcctaatggtggtccttctggcgtgttcttctccaggacagaagaagcaccattttga

SEQ ID NO: 12 - protein, 298 aa, Quillaja saponaria
  misc_feature: An oxidoreductase enzyme capable of enhancing the activity of a fucosyltransferase (QsFucSyn)
MAEATQRYAVVTGSNKGIGFGICKQLASKGIKVVLTARDEKRGLEAVEKLKEISLAGHVVFHQLDVSDPASVTSLEDFIKTQFGKLDILVNNAGITGTTVDADALAASGFGTGGERKPIDWSKLVIQTYESVEKAFNTNYYGGKRMTEALIPLLQLSDSPRIVNVSSAMGQLENIPSGWAKEVLTDVDNLTEGKLDEVSTQFLKDFKEGSLETKGWPSLMSSYIVSKAVLNAYTRILAKKYPAFCINCVDPGYVKTDINHHTGQLSVDEGAESPVRLALLPNGGPSGVFFSRTEEAPF

SEQ ID NO: 13 - DNA, 1197 bp, Quillaja saponaria
  misc_feature: A nucleic acid molecule comprising a nucleotide which encodes the enzyme according to SEQ ID NO 14.
atgccaactttgtacaaaaaagcaggcttaatggcgtcggcgtcaagggtagatctggatggtaatcagataaagccgatgacaatttgcatgatcggtgccggtgggttcattgggtcccacctctgcgagaagattatggcggagacaccgcacaaggttctggcattagatgtctacaatgacaagatcaagcacttactggagccggattctcttcaatggaaagatcgcatccaattccaccgcatcaacattaagcacgattcgaggctcgaaggtctcatcaagatggcagatctgactataaatctggctgctatttgtactcccgcggattacaacacccgtcctctggacacaatttatagcaatttcattgacgctcttcctgtggtaaagtactgttcggagaataacaagcgtctcattcatttctctacgtgtgaagtgtatgggaaaacgattgggagctttctcccaaaagacagccctcttctaaaggatcctgaatattttgttcttaaagaagatgcctccccatgcatatttggtcctattgaaaagcagagatggtcctacgcatgtgcaaagcaattgattgagaggctggtttatgctgagggtgctgagaatggccttgagttcactattgtgcgacctttcaattggattggccccagaatggatttcatacctggcattgatggtccaagtgaaggtgttccacgggttctggcatgctttagtaacaatctccttcgcggtgagccactcaaactcgttgatggtggccaatcccagagaacttttgtttatatcaaggatgcaattgaagctgttttgttgatgattgaaaaccctgccagggctaatggtcatatttttaatgtgggtaaccctcacaatgaagttacagttcggcaacttgctgaaatgatgaccgaggtctattctaaggtaagtggagaaccgtctcttgaggtgcctaccattgatgtaagctccaaagaattttatggtgaaggatacgatgatagtgacaagagaattcctgacatgaccataatcaacaggcaacttggctggaaccctaagacatcgctctgggatcttcttgagtcgaccctcacctatcaacataggacatatgcagaagctattaagaaatcaattgcgaaaccagttgccagctag

SEQ ID NO: 14 - protein, 398 aa, Quillaja saponaria
  misc_feature: An enzyme capable of enhancing the activity of an apiosyltransferase (QsAXS1).
MPTLYKKAGLMASASRVDLDGNQIKPMTICMIGAGGFIGSHLCEKIMAETPHKVLALDVYNDKIKHLLEPDSLQWKDRIQFHRINIKHDSRLEGLIKMADLTINLAAICTPADYNTRPLDTIYSNFIDALPVVKYCSENNKRLIHFSTCEVYGKTIGSFLPKDSPLLKDPEYFVLKEDASPCIFGPIEKQRWSYACAKQLIERLVYAEGAENGLEFTIVRPFNWIGPRMDFIPGIDGPSEGVPRVLACFSNNLLRGEPLKLVDGGQSQRTFVYIKDAIEAVLLMIENPARANGHIFNVGNPHNEVTVRQLAEMMTEVYSKVSGEPSLEVPTIDVSSKEFYGEGYDDSDKRIPDMTIINRQLGWNPKTSLWDLLESTLTYQHRTYAEAIKKSIAKPVAS

SEQ ID NO: 15 - DNA, 1275 bp, Avena strigosa
  misc_feature: A nucleic acid molecule comprising a nucleotide which encodes the enzyme according to SEQ ID NO 16.
atggcgcccgagaaaatgcccgaggaggacgaggaaatcgtcgccggggtcgtcgcagggaagatcccctcctacgtgctcgagaccaggctaggcgactgccgcagggcagccgggatccgccgcgaggcgctgcgccggatcaccggcagggagatcgacggccttcccctcgacggcttcgactacgactcgattctcggacagtgctgcgagatgcccgtcgggtacgtgcagctgccggtcggcgtcgcggggccgctcgtcctcgacggccgccgcatatacgtcccgatggccaccacggagggctgcctaatcgccagcaccaaccgcggatgcaaggccattgccgagtccggaggcgcatccagcgtcgtgtaccgcgacgggatgacccgcgcccccgtagcccgcttcccctccgcacgacgcgccgcagagctcaagggcttcctggagaatccggccaactacgacaccctgtccgtggtctttaacagatcaagcagatttgcaaggctgcagggggtcaagtgcgccatggctgggaggaacttgtacatgaggttcacctgcagcaccggggatgccatggggatgaacatggtctccaagggcgtccaaaatgtgctcgactatctgcaggaggacttccctgacatggacgttgtcagcatctcaggcaacttttgttccgacaagaaatcagctgctgtaaactggattgaaggccgtggaaagtccgtggtttgtgaggcagtaatcagagaggaagttgtccacaaggttctcaagaccaacgttcagtcactcgtggagttgaatgtgatcaagaaccttgctggctcagcagttgctggtgctcttgggggtttcaacgcccacgcaagcaacatcgtaacggctatcttcattgccactggtcaggatcctgcacagaatgtggagagctcacagtgtatcactatgttggaagctgtaaatgatggcagagaccttcacatctccgttacaatgccatctatcgaggtgggcacagttggtggaggcacgcagctggcctcacagtcggcctgcttggacctactgggcgtcaaaggcgccaacagggaatctccggggtcgaacgctaggctgctggccacggtggtggctggtgccgtcctagctggggagctgtccctcatctccgcccaagctgccggccatctggtccagagccacatgaaatacaacagatccagcaaggacatgtccaagatcgcctgctga

SEQ ID NO: 16 - protein, 424 aa, Avena strigosa
  misc_feature: AstHMGR (Avena strigosa truncated HMG-CoA reductase) translated nucleotide sequence
MAPEKMPEEDEEIVAGVVAGKIPSYVLETRLGDCRRAAGIRREALRRITGREIDGLPLDGFDYDSILGQCCEMPVGYVQLPVGVAGPLVLDGRRIYVPMATTEGCLIASTNRGCKAIAESGGASSVVYRDGMTRAPVARFPSARRAAELKGFLENPANYDTLSVVFNRSSRFARLQGVKCAMAGRNLYMRFTCSTGDAMGMNMVSKGVQNVLDYLQEDFPDMDVVSISGNFCSDKKSAAVNWIEGRGKSVVCEAVIREEVVHKVLKTNVQSLVELNVIKNLAGSAVAGALGGFNAHASNIVTAIFIATGQDPAQNVESSQCITMLEAVNDGRDLHISVTMPSIEVGTVGGGTQLASQSACLDLLGVKGANRESPGSNARLLATVVAGAVLAGELSLISAQAAGHLVQSHMKYNRSSKDMSKIAC

SEQ ID NO: 17 - DNA, 2277 bp, Quillaja saponaria
  misc_feature: A nucleic acid molecule comprising a nucleotide which encodes the enzyme according to SEQ ID NO 18.
atgtggaggctgaagatagcagaaggtggttccgatccatatctgttcagcacaaacaacttcgtgggtcgccagacatgggagttcgaaccggaggccggcacacctgaggagcgagcagaggtcgaagctgcccgccaaaacttttacaacaaccgttaccaggtcaagccctgtgacgacctcctttggagatatcagttcctgagagagaagaatttcaaacaaacaataccgcctgtcaaggttgaagatggccaagaaattacttatgagatggccacaacctcaatgcagagggcggcccgtcacctatcagccttgcaggccagcgatggccattggccagctcaaattgctggccccttgttcttcatgccacccttggtcttttgtgtgtacattactgggcatcttaatacagtattcccatctgaacatcgcaaagaaatccttcgttacatgtactatcaccagaacgaagatggtgggtggggactgcacatagagggtcacagcaccatgttttgcacagcactcaactacatttgtatgcgtatccttggggaaggaccagaggggggtcaagacaatgcttgtgccagagcacgaatgtggattcttgatcatggtggtgtaacacatattccatcttggggaaagacctggctttcgatacttggtctatttgagtggtctggaagcaatccaatgcctccagagttttggatccttccttcatttcttcctatgcatccagcaaaaatgtggtgctattgccggatggtttacatgcccatgtcttatttatatgggaaaaggtttgttggcccaatcacgcctctcattgttcagttaagagaggaaatacacactcaaaattaccatgaaatcaactggaagtcagtccgccatctatgtgcaaaggaggatatctactatccccatccactcatccaagatttgatttgggacagtttgtacatactaacggagcctcttctcactcgctggcccttgaacaagttggtgcgggagagggctctccaagtaacaatgaagcatatccactatgaagatgaaaatagtcgatacataaccattggatgtgtggaaaaggtgttatgtatgcttgcttgttgggttgatgatccaaatggagatgctttcaagaagcaccttgctcgagtcccagattacgtatgggtctctgaagatggaattactatgcagagttttggtagtcaagaatgggatgctggctttgccgtccaggctctgcttgcttctaatcttaccgaggaacttggccctgctcttgccaaaggacatgacttcataaagcaatctcaggttaaggacaatccttcaggtgacttcaaaagcatgtatcgtcacatttctagaggatcatggaccttctctgaccaagatcatggatggcaagtttctgattgcactgcagaaggtctgaagtgttgcctgcttttgtcgatgttgccaccagaaattgttggtgaaaaaatggaaccacaaaggctatttgattctgtcaatgtgctgctctctctacagagcaaaaaaggtggtttagctgcctgggagccagcaggggcgcaagattggttggaattactcaatcccacagaattttttgcggacattgtcgttgagcatgaatatgttgaatgtactggatcagcaattcaggcattagttttgttcaagaagctgtatccggggcacaggaaaaaagagattgacagtttcattacaaatgctgtccggttccttgagaatacacaaacggcagatggctcttggtatggaaactggggagtttgcttcacctatggttgttggttcgcactgggagggctagcagcagctggcaagacttacaacaactgtcctgcaatacgcaaagctgttaatttcctacttacaacacaaagagaagacggtggttggggagaaagctatctttcaagcccaaaaaagatatatgtacccctggaaggaagccgatcaaatgtggtacatactgcatgggctatgatgggtctaattcatgctgggcaggctgaaagagactcaactcctcttcatcgtgcagcaaagttgatcatcaattatcaactagaaaatggcgattggccgcaacaggaaatcactggagtattcatgaaaaactgcatgttacattaccctatgtacagaaacatctacccaatgtgggctcttgcagaataccggaggcgggttccattgccttaa

SEQ ID NO: 18 - protein, 758 aa, Quillaja saponaria
  misc_feature: An enzyme involved in making beta-amyrin from 2,3-oxidosqualene (QsbAS)
MWRLKIAEGGSDPYLFSTNNFVGRQTWEFEPEAGTPEERAEVEAARQNFYNNRYQVKPCDDLLWRYQFLREKNFKQTIPPVKVEDGQEITYEMATTSMQRAARHLSALQASDGHWPAQIAGPLFFMPPLVFCVYITGHLNTVFPSEHRKEILRYMYYHQNEDGGWGLHIEGHSTMFCTALNYICMRILGEGPEGGQDNACARARMWILDHGGVTHIPSWGKTWLSILGLFEWSGSNPMPPEFWILPSFLPMHPAKMWCYCRMVYMPMSYLYGKRFVGPITPLIVQLREEIHTQNYHEINWKSVRHLCAKEDIYYPHPLIQDLIWDSLYILTEPLLTRWPLNKLVRERALQVTMKHIHYEDENSRYITIGCVEKVLCMLACWVDDPNGDAFKKHLARVPDYVWVSEDGITMQSFGSQEWDAGFAVQALLASNLTEELGPALAKGHDFIKQSQVKDNPSGDFKSMYRHISRGSWTFSDQDHGWQVSDCTAEGLKCCLLLSMLPPEIVGEKMEPQRLFDSVNVLLSLQSKKGGLAAWEPAGAQDWLELLNPTEFFADIVVEHEYVECTGSAIQALVLFKKLYPGHRKKEIDSFITNAVRFLENTQTADGSWYGNWGVCFTYGCWFALGGLAAAGKTYNNCPAIRKAVNFLLTTQREDGGWGESYLSSPKKIYVPLEGSRSNVVHTAWAMMGLIHAGQAERDSTPLHRAAKLIINYQLENGDWPQQEITGVFMKNCMLHYPMYRNIYPMWALAEYRRRVPLP

SEQ ID NO: 19 - DNA, 1443 bp, Quillaja saponaria
  misc_feature: A nucleic acid molecule comprising a nucleotide which encodes the enzyme according to SEQ ID NO 20
atggagcacttgtatctctcccttgtgctcctgtttgtttcctcaatctccctctccctcttcttcctgttctacaaacacaaatctatgttcaccggggccaacctaccacctggtaaaatcggttacccattgatcggagagagcttggagttcttgtccacgggatggaagggccacccggagaaattcatcttcgatcgcatgagcaagtactcatcccaaatcttcaagacctcgattttaggggaaccaacggcggtgttcccgggagccgtatgcaacaagttcctcttctccaacgagaacaagctggtgaatgcatggtggcctgcctccgtggacaagatctttccttcctcactccagacatcctccaaagaagaggccaagaagatgaggaagttgcttcctcagtttctcaagcccgaagctctgcaccgctacattggtattatggattctattgcccagagacactttgccgatagctgggaaaacaaaaaccaagtcattgtctttcctctagcaaagaggtatactttctggctggcttgccgtttgttcattagcgtcgaggatccgacccacgtatccagatttgctgacccgttccaacttttggccgccggaatcatatcaatcccaatcgacttgccagggacaccgttccgcaaggcaatcaatgcgtcccagttcatcaggaaggaattgttggccatcatcaggcagagaaagatcgatttgggtgaagggaaggcatctccgacgcaggacatactgtctcacatgttgctcacatgcgacgagaacggacaatacatgaatgaattggacattgccgacaagattcttggcttgttggtcggcggacatgacactgccagtgccgcttgcactttcattgtcaagttcctcgctgagcttccccacatttatgaacaagtctacaaggagcaaatggagattgcaaaatcaaaagtgccaggagagttgttgaattgggaggacatccaaaagatgaaatattcgtggaacgtagcttgtgaagtgatgagacttgcccctccactccaaggagctttcagggaagccattactgacttcgtcttcaacggtttctccattccaaaaggctggaagttgtactggagcgcaaattccacccacaaaagtccggattatttccctgagcccgacaagttcgacccaactagattcgaaggaaatggacctgcgccttacacctttgttccatttgggggaggacccaggatgtgcccgggcaaagagtatgcccgattggaaatacttgtgttcatgcataacttggtgaagaggttcaagtgggagaaattggttcctgatgaaaagattgtggttgatccaatgcccattccagcaaagggtcttcctgttcgcctttatcctcacaaagcttga

SEQ ID NO: 20 - protein, 480 aa, Quillaja saponaria
  misc_feature: An enzyme involved in making Oleanolic acid from beta-amyrin (QsCYP716-C-28)
MEHLYLSLVLLFVSSISLSLFFLFYKHKSMFTGANLPPGKIGYPLIGESLEFLSTGWKGHPEKFIFDRMSKYSSQIFKTSILGEPTAVFPGAVCNKFLFSNENKLVNAWWPASVDKIFPSSLQTSSKEEAKKMRKLLPQFLKPEALHRYIGIMDSIAQRHFADSWENKNQVIVFPLAKRYTFWLACRLFISVEDPTHVSRFADPFQLLAAGIISIPIDLPGTPFRKAINASQFIRKELLAIIRQRKIDLGEGKASPTQDILSHMLLTCDENGQYMNELDIADKILGLLVGGHDTASAACTFIVKFLAELPHIYEQVYKEQMEIAKSKVPGELLNWEDIQKMKYSWNVACEVMRLAPPLQGAFREAITDFVFNGFSIPKGWKLYWSANSTHKSPDYFPEPDKFDPTRFEGNGPAPYTFVPFGGGPRMCPGKEYARLEILVFMHNLVKRFKWEKLVPDEKIVVDPMPIPAKGLPVRLYPHKA

SEQ ID NO: 21 - DNA, 1506 bp, Quillaja saponaria
  misc_feature: A nucleic acid molecule comprising a nucleotide which encodes the enzyme according to SEQ ID NO 22.
atgatatataataatgatagtaatgataatgaattagtaatcagctcagttcagcaaccatccatggatcctttcttcatttttggcttacttctcttggctctctttctctctgtttcttttcttctctacctttcccgtagagcctatgcttctctccccaaccctccgccggggaagctcggcttccccgtcgtcggcgagagtctcgaatttctctccacccgacgcaaaggtgttcctgagaaattcgtcttcgacagaatggccaaatactgtcgggatgtctttaagacatcaatattgggagcaaccaccgccgtcatgtgcggcaccgccggtaacaaattcttgttctccaacgagaaaaaacacgtcactggttggtggccgaaatctgtagagctgattttcccaacctcacttgagaaatcatccaacgaagaatccatcatgatgaaacaattccttcccaacttcttgaaaccagaacctttgcagaagtacatacccgttatggacataattacccaaagacacttcaatacaagctgggaaggacgcaacgtggtcaaagtgtttcctacggctgccgaattcaccacgttgctggcttgtcgggtattcctcagtgttgaggatcccattgaagtagccaagatttcagagccatttgaaatcttagctgctgggtttctttcaatacccataaatcttccgggtaccaaattaaataaagcggttaaggcagcggatcagattagagacgcaattgtacagattttgaaacggagaagggttgaaattgcggagaataaagcaaatggaatgcaagatatagcgtccatgttgttgacgacaccaactaatgctgggttttatatgaccgaggctcacatttctgagaaaattttgggtatgattgttggtggccgtgatactgctagtactgttatcaccttcatcatcaagtatttggcagagaatcctgaaatttataataaggtctatgaggagcaaatggaagtggtaaagtcaaagaaaccaggtgagttgctgaactgggaagatgtgcagaaaatgaagtactcttggtgcgtagcatgtgaagctatgcgacttgctcctcctgttcaaggtggtttcaaggtggccattaatgactttgtgtattctgggttcaacattcgcaagggttggaagttatattggagtgccattgcaacacacatgaatccagaatatttcccagaacctgagaaattcaacccctcaaggtttgaagggaagggaccagtaccttacagcttcgtacccttcggaggcggacctcggatgtgtcccgggaaagagtattcccggctggaaacacttgttttcatgcatcatttggtgacgaggtacaattgggagaaagtgtatcccacagagaagataacagtggatccaatgccattccctgtcaacggcctccccattcgccttattcctcacaagcaccaatga

SEQ ID NO: 22 - protein, 501 aa, Quillaja saponaria
  misc_feature: An enzyme involved in making Echinocystic acid from Oleanolic acid (QsCYP716-C-16alpha)
MIYNNDSNDNELVISSVQQPSMDPFFIFGLLLLALFLSVSFLLYLSRRAYASLPNPPPGKLGFPVVGESLEFLSTRRKGVPEKFVFDRMAKYCRDVFKTSILGATTAVMCGTAGNKFLFSNEKKHVTGWWPKSVELIFPTSLEKSSNEESIMMKQFLPNFLKPEPLQKYIPVMDIITQRHFNTSWEGRNVVKVFPTAAEFTTLLACRVFLSVEDPIEVAKISEPFEILAAGFLSIPINLPGTKLNKAVKAADQIRDAIVQILKRRRVEIAENKANGMQDIASMLLTTPTNAGFYMTEAHISEKILGMIVGGRDTASTVITFIIKYLAENPEIYNKVYEEQMEVVKSKKPGELLNWEDVQKMKYSWCVACEAMRLAPPVQGGFKVAINDFVYSGFNIRKGWKLYWSAIATHMNPEYFPEPEKFNPSRFEGKGPVPYSFVPFGGGPRMCPGKEYSRLETLVFMHHLVTRYNWEKVYPTEKITVDPMPFPVNGLPIRLIPHKHQ

SEQ ID NO: 23 - DNA, 1524 bp, Quillaja saponaria
  misc_feature: A nucleic acid molecule comprising a nucleotide which encodes the enzyme according to SEQ ID NO 24.
atgtggttcacagtaggattggtcttggttttcgccctattcatacgtctctacagcagtctgtggttgaagcctcgtgcaactcggattaagcttagcaatcaaggaattaaaggtccaaaaccagcatttcttctgggtaatgttgcagagatgagaagatttcaatctaagcttccaaaatctgaactcaaacaaggccaagtttctcatgattgggcttctaaatctctgtttccatttttcagtctttggtcccagaaatacggaaatacgttcgtgttctcattggggaacatacaggtgctctatgtttctgatcatgagttggtgaaagaaattaatcagaatacctctttagatttgggcaaacccaagtacctgcagaaggagcgtggccctttgctgggacaaggtattttgacctccaatggacagctttgggcgtaccagagaaaaatcatgactcctgaactctacaaggagaaaatcaagggcatgtgcgagttgatggtggaatctgtagcttggttggttgaggaatggggaacgaagatccaagctgagggtggggcagcagacattagaatagacgaggatcttagaagcttctctggtgatgtaatttcaaaagcttgttttgggagctgctatgccggagggagggaaatctttcttaggctcagagctcttcaacaccaaattgcttccaaagccttactcatgggcttccctggattaaagtacctgcccattaagagcaacagagagatatggagattggagaaggagatcttccagctgattatgaagctggctgaagatagaaaaaaagaacaacatgagagagacctattacagattataattgagggagctaaaagtagtgatctgagttcggaagcaatggcaaaattcattgtggacaactgcaagaatgtctacttggctggccatgaaactactgcaatgtctgctggttggactttgcttctcttggctaatcatcctgagtggcaagcccgtgtccgtgatgagattttacaagtcaccgagggccgcaatcctgattttgacatgctgcacaagatgaaactgttaacaatggtaattcaggaggcactgcgactctacccaacagtcatattcatgtcaagagaagcattggaagatattaatgttggaaacatccaagttccaaaaggtgttaacatatggatacctgtggtaaatcttcaaagggacacaacggtatggggtgcagacgcaaacgagtttaatcctgaaaggtttgccaatggagttaacaattcatgcaaggttccacaactttacctaccatttggagctggacctcgcatttgtcctggaattaatctggccatgactgagatcaagatacttctgtgtatcctgctcaccaagttttcgttttcagtttcacccaactatcgccactcaccggtgtttaaattggtgcttgagcctgaaaatggaatcaatgtcatcatgaagaagctctaa

SEQ ID NO: 24 - protein, 507 aa, Quillaja saponaria
  misc_feature: An enzyme involved in making Quillaic acid from Echinocystic acid (QsCYP714-C-23).
MWFTVGLVLVFALFIRLYSSLWLKPRATRIKLSNQGIKGPKPAFLLGNVAEMRRFQSKLPKSELKQGQVSHDWASKSLFPFFSLWSQKYGNTFVFSLGNIQVLYVSDHELVKEINQNTSLDLGKPKYLQKERGPLLGQGILTSNGQLWAYQRKIMTPELYKEKIKGMCELMVESVAWLVEEWGTKIQAEGGAADIRIDEDLRSFSGDVISKACFGSCYAGGREIFLRLRALQHQIASKALLMGFPGLKYLPIKSNREIWRLEKEIFQLIMKLAEDRKKEQHERDLLQIIIEGAKSSDLSSEAMAKFIVDNCKNVYLAGHETTAMSAGWTLLLLANHPEWQARVRDEILQVTEGRNPDFDMLHKMKLLTMVIQEALRLYPTVIFMSREALEDINVGNIQVPKGVNIWIPVVNLQRDTTVWGADANEFNPERFANGVNNSCKVPQLYLPFGAGPRICPGINLAMTEIKILLCILLTKFSFSVSPNYRHSPVFKLVLEPENGINVIMKKL

SEQ ID NO: 25 - DNA, 2142 bp, Quillaja saponaria
  misc_feature: A nucleic acid molecule comprising a nucleotide which encodes the enzyme according to SEQ ID NO 26.
atgaaatccccctctaacccaaatcagaaacccatcctccacacttgtacaattcagcagcctcgtgctacccttaacaaaattcatagtcttattcatttctcagccatacttgtcctattttattaccggataacccgtctattcttcaccgacgatttcaaggtacccaagttactatggactctaatgacaatctccgagttcattcttgccttcatttgggttctcatccaacctttccggtggcgaccggtgtcccgttccgtcataccagagaatatgccgaaggacatcagtttgccggcggtggacgtgtttgtatgcacagctgaccctcaaaaagaacccacagtggaggtgatgaacacaattttatcagccatggctttagactacccggcggagaagctcgccgtgtatctttccgatgatgggggttctgctgtcaccttatatgctataaaagaagcttgttgttttgctaagatgtggcttccgttttgtaacaagtatgggatcaaatcaaggtgtcccgaggcttatttttcaaagcttgccgctgacgagtggcttcaccggagtgtggaattcgtggcagaagaaaaggaggtcaaggctaattatgaagagttcaagagaaatgtgcagaaatttggtgagcaacaagaaaacagtcgtgttgtgcatgatcgtcaccctcatgttgagattatacacaataattggaataacgaagaccaagctcatgagatgccactccttgtttatgtctctcgtgaaagaagaccatctcaccatcctcgattcaaagctggagctcttaacacccttcttcgagtttctggcatcatcagcaacagcccctacatactggttctagactgtgacatgtactgcaatgacccaacctcagctagacaagcaatgtgcttccatcttgatccccaactgtctaaaaatcttgcttttgtacaattccctcaaatattctataacgctagtaagaatgacgtctatgatgcccaagtcagggcggcataccagacaaagtggcagggtatggatggacttcaaggaccaattttttctggcactggcttttacttaaagaggaaggcaatgtatggaaaccctgatcaagatgataattgtctactcaagccatataagaaatttggcatgtctggagaatttgtagaatcacttaaggtccttaacgaacaagatggtacccagaagaaattattggatggatttttacaagaggccaaactattggcctcgtgtgcctatgaaacaaagacaagttggggtaaagagattggattctcatatgactgtttaatagagagcactttcactggttatcttttgcactgcagagggtggatatctgtttatctttatcccaagagaccatgttttttaggatgctgtcctactgatatgaaggatgccatggttcaatataccaagtggatgtctgagctattttcaattgctatctcaagattcaatcctctgctctatggggtggcaagaatgtccattcttcaaagcctgtgttatggatcctttacactggcgcctattttgtcatttcctttgttcttatatggaacggttcctcaattatgcctcttgaaaggcatatctttgtttccaaaggtttcggacccatggtttgctgtgtttgcagctatctttgtatcctccctgtgtcaacactggttcgaggtcctctcttgtgatggtacatttacgacttggtgtaatgaacagcggagttggcttataaagtcggtttccggtagtttgtttggagttgtgggcgcaatcttgcagcggctaggcttgaagacaaagtttagtttatcaaacaaagccatggacaaagaaaagctggagaaatatgaaaagggtaaatttaatttccaaggggctgccatgttcatggttcctgtgtctattttagtcatactgaacacattttgcttcctcggtgggttttggaaagtgatcataatgaagaatatcctggacatgtttggacaactttctctctctgcctacgttctggttctcagttgtccagttcttgaagggatgttaactagaatcagcaagaaaatggtctga

SEQ ID NO: 26 - protein, 713 aa, Quillaja saponaria
  misc_feature: An enzyme involved in making QA-mono from Quillaic acid (QsCSL1).
MKSPSNPNQKPILHTCTIQQPRATLNKIHSLIHFSAILVLFYYRITRLFFTDDFKVPKLLWTLMTISEFILAFIWVLIQPFRWRPVSRSVIPENMPKDISLPAVDVFVCTADPQKEPTVEVMNTILSAMALDYPAEKLAVYLSDDGGSAVTLYAIKEACCFAKMWLPFCNKYGIKSRCPEAYFSKLAADEWLHRSVEFVAEEKEVKANYEEFKRNVQKFGEQQENSRVVHDRHPHVEIIHNNWNNEDQAHEMPLLVYVSRERRPSHHPRFKAGALNTLLRVSGIISNSPYILVLDCDMYCNDPTSARQAMCFHLDPQLSKNLAFVQFPQIFYNASKNDVYDAQVRAAYQTKWQGMDGLQGPIFSGTGFYLKRKAMYGNPDQDDNCLLKPYKKFGMSGEFVESLKVLNEQDGTQKKLLDGFLQEAKLLASCAYETKTSWGKEIGFSYDCLIESTFTGYLLHCRGWISVYLYPKRPCFLGCCPTDMKDAMVQYTKWMSELFSIAISRFNPLLYGVARMSILQSLCYGSFTLAPILSFPLFLYGTVPQLCLLKGISLFPKVSDPWFAVFAAIFVSSLCQHWFEVLSCDGTFTTWCNEQRSWLIKSVSGSLFGVVGAILQRLGLKTKFSLSNKAMDKEKLEKYEKGKFNFQGAAMFMVPVSILVILNTFCFLGGFWKVIIMKNILDMFGQLSLSAYVLVLSCPVLEGMLTRISKKMV

SEQ ID NO: 27 - DNA, 2124 bp, Quillaja saponaria
  misc_feature: A nucleic acid molecule comprising a nucleotide which encodes the enzyme according to SEQ ID NO 28.
atggcgaccgtctcctccctccacacttgcactgtacagcaaccccgtgcagccattaatcgaattcacattttcttacactttattgccatacttttcctcttttactaccgggtcaccggtcttttctatgacaatgcagtacccactttagcttggtctctaatgaccttagctgagttgattttcgccttcgtttgggtgctcagccaagccttccggtggcgcccggtgttgcgttcagttattcctgagaggattcccaaagatgtacgattgcccgcggtggatatcttaatttgtacggctgacccattaaaggaaccgacggtggaggtgatgaacactgtcttgtccgccatggcattggactatcctgcggagaatctggctgtatatctttctgatgacgggggttctccggtcaccttatttgctatgaagcaagtgggtccgtttgctaagctgtggcttccgttttgcaacaagtacggaatcaaaacaaggcatcctgagtcttttttctcggcatttgcggatgacgaaaggcttcaccggagtgatgaattcagggcagaggaggaggcgatcaaggacaaatatgaagaatttaagagaactatagagaaatatggtggagaaggaaaaaatagtcatgttgtacaagaccggcctcctcatgtggagattatacatgacactaggaagattagagagaacagtgaagaccaagctgtgcctcttcttgtctacgtctctcgtgagaaaagaccatcctacaattctcggttcaaagcaggagctctgaacacccttcttcgagtttctggggtaatcagcaatagcccatatgtattggtgttagactgtgacatgtactgcaatgatccaacatcagctagacaagcaatgtgcttccatcttgatccacaaatgtctcgcactctctcttttgtacaattcccccaggttttctacaatgttagtaaaaatgatatctatgatggccaagctagggcagcctttaagacaaagtggcaaggtatggatggactacgtgggccactgctttctggtactggcttttatttgaagaggaagtccttgtatggaagtccaaaccaagaagatgattgtttacttgagccccataagaattttggaaagtgtgacaagctcatagaatcagtaaaggtcatttatgaacgtgatgtttcaataaaggcagattcatcagatgccattttgcaagatgccaaacaattagcatcttgtccctatgaaacaaacacaagctggggcaaagaggttgggttctcgtatgactgcttattagagagtacattcacaggttatctgttgcactgcagagggtggacatcagtttatctttatccaaagaagccatgtttcttagggtgtactccagttgatatgaaggaagccatggttcagtatacgaagtggatttctgaattatttttacttgctatctcaagattcaaccctctgacatttgggatatccagaatgtccattctccagagcatgtgttacggataccttacaatcatgcccattttatctgttgctatgatcttctatgccacagttcctcaattgtgcctcttgagaggcgtacctctgtttcccaaggtttcagacccatggtttgcagtgttcctagcaatatttgtgtcctccctctgtcagcacttaattgaagtcctcacgagtgatggcacgctcaagacttggtggaatgaacaaagaaattgggtgataaagtctggttccggtagcgtatttggagctctgagtggaatattgaagtggtttggcatgaagattaaatttggtttatcaaacaaagccgtggacaaagaaaagcttgagaaatatgaaaagggtaagtttgatttccaaggggctgccatgtttatggttcccttaactatatcagtcatcttgaacacattatgccttatcggtggtttatggagagtaatcacacttaaaaacttcgaagagatgtcagggcagttcatcatctccttgtactttctagctctcagctatccaattcttgaagggttactaagaaaaggcaagggaaaggcctaa

SEQ ID NO: 28 - protein, 707 aa, Quillaja saponaria
  misc_feature: An enzyme involved in making QA-mono from Quillaic acid (QsCslG2).
MATVSSLHTCTVQQPRAAINRIHIFLHFIAILFLFYYRVTGLFYDNAVPTLAWSLMTLAELIFAFVWVLSQAFRWRPVLRSVIPERIPKDVRLPAVDILICTADPLKEPTVEVMNTVLSAMALDYPAENLAVYLSDDGGSPVTLFAMKQVGPFAKLWLPFCNKYGIKTRHPESFFSAFADDERLHRSDEFRAEEEAIKDKYEEFKRTIEKYGGEGKNSHVVQDRPPHVEIIHDTRKIRENSEDQAVPLLVYVSREKRPSYNSRFKAGALNTLLRVSGVISNSPYVLVLDCDMYCNDPTSARQAMCFHLDPQMSRTLSFVQFPQVFYNVSKNDIYDGQARAAFKTKWQGMDGLRGPLLSGTGFYLKRKSLYGSPNQEDDCLLEPHKNFGKCDKLIESVKVIYERDVSIKADSSDAILQDAKQLASCPYETNTSWGKEVGFSYDCLLESTFTGYLLHCRGWTSVYLYPKKPCFLGCTPVDMKEAMVQYTKWISELFLLAISRFNPLTFGISRMSILQSMCYGYLTIMPILSVAMIFYATVPQLCLLRGVPLFPKVSDPWFAVFLAIFVSSLCQHLIEVLTSDGTLKTWWNEQRNWVIKSGSGSVFGALSGILKWFGMKIKFGLSNKAVDKEKLEKYEKGKFDFQGAAMFMVPLTISVILNTLCLIGGLWRVITLKNFEEMSGQFIISLYFLALSYPILEGLLRKGKGKA

SEQ ID NO: 29 - DNA, 1479 bp, Quillaja saponaria
  misc_feature: A nucleic acid molecule comprising a nucleotide which encodes the enzyme according to SEQ ID NO 30.
atggtggagtctccagcagatcatgatgtgctcaaaatcattgtccttccatgggtaacctcaggtcacatgattcccatggtagatgcagccagactatttgctatgcatggtgcagatgttaccatcatcaccaccccagctaatgcccttacattccagaaatccgtcgaccgtgatttcaattccggtcgtttaatcagaactcacacccttaaattccctgcagcagaagttggtgtacctgaaggagttgaaaacttcaacaatacttcccctgaaatgacctccaaagtctaccttggagtctcaatgctccgagaaccaacccaacaattgattgaggatctgcgtccagattgtcttatcactgatatgttctatccttgggctgtggatgttgctgacaaattaggcattccaaggctaatttttcaaggtcctggaagttttggtttgtcagctatgcattctatcaaacagtatgagccctttaagtcagtaacttcagatactgagacattcccactacctggattgccgcataaggtagagatgacaaggttgcagataccaaaatgggttcgtgagccaaatgggtacactcaattgatgggcagggtaaaagattcggagagaagaagctatgggtcattggtgaatagcttttatgacttcgaaggcccttatgaagagcactataggaaggcaacaggacagagggtttggagcattggaccagtttcagtttgggtgaaccaagatgctgcagataaggttggaagaggacaggatcttgttgctgaagaccaaaacagctggttgaattggctcaattccaaagagaaaaactctgttctgtatgtaagttttgggagcatggccaagttcccatctgctcagcttcttgaaatagctcatgggcttgaagcttcaggtcatagtttcatctgggttgtcagaaaagttgacggggatgatgatgtagacgtgtggcttccagattttgagaagaaaatgaaagagaacaacaagggtttcatcataaggaattgggcaccacaattgctcatattggaccatccagcaattggaggtttgctgaatcacagtggatggaattcagtactggaaggtgctacagcaggcttgccaatgatcacttggcctctgtatgccgagcatttttacaatgaaaggttggttctagatgtgttgaaaattggagtaccagttggggtgaaggagtggaagaacttgcatgaggtgggtgagttggtgagaagggatgcaattgccaaggcaattaaattgttaatgggtagtggagaagaagctgaggtaatgaggaaaaaagccaaagagcttggtgttggagcaaagaaaggtattcaggttggaggttcttctcataccaatttgatagcagtgattgatgagttaaagtcactaaagaaatcaagaattcagggtgtctga

SEQ ID NO: 30 - protein, 492 aa, Quillaja saponaria
  misc_feature: An enzyme involved in making QA-Di from QA-Mono (Qs-3-O-GalT).
MVESPADHDVLKIIVLPWVTSGHMIPMVDAARLFAMHGADVTIITTPANALTFQKSVDRDFNSGRLIRTHTLKFPAAEVGVPEGVENFNNTSPEMTSKVYLGVSMLREPTQQLIEDLRPDCLITDMFYPWAVDVADKLGIPRLIFQGPGSFGLSAMHSIKQYEPFKSVTSDTETFPLPGLPHKVEMTRLQIPKWVREPNGYTQLMGRVKDSERRSYGSLVNSFYDFEGPYEEHYRKATGQRVWSIGPVSVWVNQDAADKVGRGQDLVAEDQNSWLNWLNSKEKNSVLYVSFGSMAKFPSAQLLEIAHGLEASGHSFIWVVRKVDGDDDVDVWLPDFEKKMKENNKGFIIRNWAPQLLILDHPAIGGLLNHSGWNSVLEGATAGLPMITWPLYAEHFYNERLVLDVLKIGVPVGVKEWKNLHEVGELVRRDAIAKAIKLLMGSGEEAEVMRKKAKELGVGAKKGIQVGGSSHTNLIAVIDELKSLKKSRIQGV

SEQ ID NO: 31 - DNA, 1515 bp, Quillaja saponaria
  misc_feature: A nucleic acid molecule comprising a nucleotide which encodes the enzyme according to SEQ ID NO 32.
atggtctccggcgacgacgatgtttctcgtcggccactgaaagtttacttcattgcacacccctcacctggccatattgcccctctgaccaaaatagcccatctcttcgctgccctcggtgagcacgtgactattctcactactcccgccaatgtccacttccatgagaaatccatcgacaaaggaaaggcttccggctatcatgttaacatccacaccgttaaatttccttctaaagaggtcggtctccctgacggcatcgaaaacttctcttacgcctccgatgttgaaacagcagctaaaatttgggctggattcgccatgctacaaactgaaatggagcaatatatggagcttaacccacccgattgcatcgttgccgacatgttcacctcctggacctccgactttgctatcaaattgggaatcacaagaatcgttttcaacgtctattgtattttcacacgctgtttggaagaagccatccgatcaccggactcgccacacttgaacaaagaaatctctgataatgaaccgtttgttatcccgggtctaccagaccccataacaattacccgagctcaactgcccgacggtaccttttctcccatgaaagaactagctagaacagctgagttgaagagctttggaatggtgatcaacgggttttccgaactcgaaaccgattacatcgagcattacaagaaaatcatgggtcacaaacggatttggcatgtcggaccccttcagctaatccaccgtaacgatgaagacaaaattcagaggagccacaagacagcggtgctgagtgataacgataacgagttagtgagttggcttaactcgaagaaacccgactcagttatttacatttgcttcggtagtgcaactcgtttctctaatcaccagctctatgaaatcgcctgtggattagaagcttccgggcacccatttttgtggggcctactttgggtgccagaagatgaagataacgatgacgtgggcaacaaatggttgccagctttcgaagaaagaattaaaaaggaaaataagggaatgattttaagggggtgggctccacagatgttaatcttaaaccacccggcgatcggtggtttcatgacgcattgtggttggaatgcggtggtggaagcactttcattcggtgttccgactattacgcttccagttttctcggagcagttttatactgagagactgatatcacaagtgctcaagactggtgtggaggttggtgcagagaagtggacctatgcatttgatgcggggaaatatccggtgagtagggaaaagatagcgacggcggtgaagaagatattagacgatggagaagaggcagaaggaatgagaaagcgggccagggagatgaaagaaaaagcccaaaaaagtgttgaagaaggtggatcctcttataataatttaacggctatgattgaagatcttaaagaatttagggctaacaatggcaaggctgcacaagatcatgaatcgtga

SEQ ID NO: 32 - protein, 504 aa, Quillaja saponaria
  misc_feature: An enzyme involved in making QA-TriR or QA-TriX from QA-Di (Qs-3-O-RhaT/XylT).
MVSGDDDVSRRPLKVYFIAHPSPGHIAPLTKIAHLFAALGEHVTILTTPANVHFHEKSIDKGKASGYHVNIHTVKFPSKEVGLPDGIENFSYASDVETAAKIWAGFAMLQTEMEQYMELNPPDCIVADMFTSWTSDFAIKLGITRIVFNVYCIFTRCLEEAIRSPDSPHLNKEISDNEPFVIPGLPDPITITRAQLPDGTFSPMKELARTAELKSFGMVINGFSELETDYIEHYKKIMGHKRIWHVGPLQLIHRNDEDKIQRSHKTAVLSDNDNELVSWLNSKKPDSVIYICFGSATRFSNHQLYEIACGLEASGHPFLWGLLWVPEDEDNDDVGNKWLPAFEERIKKENKGMILRGWAPQMLILNHPAIGGFMTHCGWNAVVEALSFGVPTITLPVFSEQFYTERLISQVLKTGVEVGAEKWTYAFDAGKYPVSREKIATAVKKILDDGEEAEGMRKRAREMKEKAQKSVEEGGSSYNNLTAMIEDLKEFRANNGKAAQDHES

SEQ ID NO: 33 - DNA, 1485 bp, Quillaja saponaria
  misc_feature: A nucleic acid molecule comprising a nucleotide which encodes the enzyme according to SEQ ID NO 34.
atggtctccggcgacgacgacgtttctcgtcggccactgaaagtttactttattgcacacccctcacctggccatattgcccctctaaccaaaatagcccaactctttgctgcacgtggtgagcacgtgactattcttactactcccgccaatgtccacttccatgagaaatccatcgacaaaggaaagacttccggctatcatgttaacatccacgccgttaaatttccttctaaagaggtcggtctccccgacggcatcgaaaacttctctcacgcctccgataatgaaacagcagccaaaatttgggccggattctccatgcttcaaactgaaatggagcaatatatggaacaaaacccacccgattgcattgttgccgacatgttcaaccgctggacttccgacttcgctatcaaattgggaatcccgagaatagttttcaacgtctactgtattttcacacgctgtttggaagaagcaatcagatcacctgactcgccacacttgaaactaaactccgataatgaacagtttattattccgggtctacccgaccccataacaattacccgagctcaactgcccgacggtgccttttctgtcgtcaaagaacaagttagtgaagctgagttgaaaagcttcggaatggtgatcaacgggttttccgaactcgaaaccgaatacatcgagtattacaagaatatcatgggtcgaaaacggatttggcatgtcggaccccttcagctcatttaccaaaacgatgaccccaaagttcagaggagccagaagacagcggtcgtgagtgacaacgagttagtgagttggcttgactcgaagaaacccgactcagtgatttacatttccttcggtagtgcaattcgtttctctaataagcagctctatgaaatagcatgtggattagaagcttccggctacccatttttgtgggccttactttgggtgccagaagatgacgacgacgtgggcaacaaatggttgcctgatttcgaagaaagaataaaaagagaaaataagggaataattttcagggggtgggccccacagatgttaatcttaaaccacccggcgatcggtggtttcatgacgcattgtggttggaatgcggtggtggaagcgctttctttcggtgttccgactattacgcttccggttttctcggagcagttttatactgagagactgatatcacaagtgctcaagactggtgtcgaggtcggtgcagagaagtggacctatgcatttgatgcggggaaatatccggtgagtcgggaaaagatagcgacggcggtgaagaagatattagactgtggagaagaggcagaaggaatgagaaagcgggccagggagatgaaagaaaaagcccaaaaaagtgttgaagaaggtgggtcctcttataataatttaacggctatgattgaagatcttaaagaatttagggctaacaatggcaaggttgcatga

SEQ ID NO: 34 - protein, 494 aa, Quillaja saponaria
  misc_feature: An enzyme involved in making QA-TriR from QA-Di (Qs_0283850).
MVSGDDDVSRRPLKVYFIAHPSPGHIAPLTKIAQLFAARGEHVTILTTPANVHFHEKSIDKGKTSGYHVNIHAVKFPSKEVGLPDGIENFSHASDNETAAKIWAGFSMLQTEMEQYMEQNPPDCIVADMFNRWTSDFAIKLGIPRIVFNVYCIFTRCLEEAIRSPDSPHLKLNSDNEQFIIPGLPDPITITRAQLPDGAFSVVKEQVSEAELKSFGMVINGFSELETEYIEYYKNIMGRKRIWHVGPLQLIYQNDDPKVQRSQKTAVVSDNELVSWLDSKKPDSVIYISFGSAIRFSNKQLYEIACGLEASGYPFLWALLWVPEDDDDVGNKWLPDFEERIKRENKGIIFRGWAPQMLILNHPAIGGFMTHCGWNAVVEALSFGVPTITLPVFSEQFYTERLISQVLKTGVEVGAEKWTYAFDAGKYPVSREKIATAVKKILDCGEEAEGMRKRAREMKEKAQKSVEEGGSSYNNLTAMIEDLKEFRANNGKVA

SEQ ID NO: 35 - DNA, 1491 bp, Quillaja saponaria
  misc_feature: A nucleic acid molecule comprising a nucleotide which encodes the enzyme according to SEQ ID NO 36.
atggtctccggcgacgataccgtttcacggccactgatagtttactttattgcacacccctcacctggccatattgcccctctaaccaaaatagcccaactcttcgctgcacgtggtgagcacgtcactattcttactactcccgccaatgtccacttccatgagaaatccatcgacaaaagaaagaattccggctatcatgttaacatccacaccgttaaatttccttctaaagaggtcggtctccctgacggcatcgaaaacttctctcacgcctccgataatgaaacagcagccaaaatttgggccggattctccatgcttcaaactgaaatggagcaatatatggaacaaaacccacccgattgcatcgttgccgacatgttcaaccgctggacttccgacttcgctatcaaattgggaatcccgagaatagttttcaacgtctactgtattttcacacgctgtttggaagaagcaatcagatcacctgactcgccacacttgaaactaaactccgataatgaacagtttattattcccggtctacccgaccccataacaattacccgagctcaactccccgacggtgccttttctgtcgtcaaagaacaagttagtgaagctgagttgaaaagcttcggaatggtgatcaacgggttttccgaactcgaaactgaatacatcgagtattacaagaatatcatgggtcgcaaacggatttggcatgtcggaccccttcagctaatttaccaaaacgacgaccccaaagttcagaggagccagaagacagcggtcttgagtgacaacgagttagtgagttggcttgactcgaagaaacccgactcagtgatttacatttccttcggtagtgcaattcgtttctctaataagcagctctatgaaatcgcatgtggattagaagcttccggctacccatttttgtgggccttactttgggtgccagaagatgatgacgacgtgggcaacaaatggttgccgggtttcgaagaaagaataaaaagagaaaataagggaataattttcagggggtgggccccacagatgttaatcttaaaccacccggcgatcggtggtttcatgacgcattgtggttggaatgcggtggtggaagcactttcattcggtgttccgactattacgcttccagttttctcggagcagttttatactgagagactgatatcacaagtgctcaagactggtgtggaggttggtgcagagaagtggacctatgcatttgatgcggggaaatatccggtgagtagggaaaagatagcgacggcggtgaagaagatattagacgatggagaagaggcagaaggaatgagaaagcgggccagggagatgaaagaaaaagcccaaaaaagtgttgaagaaggtggatcctcttataataatttaacggctatgattgaagatcttaaagaatttagggctaacaatggcaaggctgcaatgaaatcatga

SEQ ID NO: 36 - protein, 496 aa, Quillaja saponaria
  misc_feature: An enzyme involved in making QA-TriR from QA-Di (DN20529_c0_g2_i8).
MVSGDDTVSRPLIVYFIAHPSPGHIAPLTKIAQLFAARGEHVTILTTPANVHFHEKSIDKRKNSGYHVNIHTVKFPSKEVGLPDGIENFSHASDNETAAKIWAGFSMLQTEMEQYMEQNPPDCIVADMFNRWTSDFAIKLGIPRIVFNVYCIFTRCLEEAIRSPDSPHLKLNSDNEQFIIPGLPDPITITRAQLPDGAFSVVKEQVSEAELKSFGMVINGFSELETEYIEYYKNIMGRKRIWHVGPLQLIYQNDDPKVQRSQKTAVLSDNELVSWLDSKKPDSVIYISFGSAIRFSNKQLYEIACGLEASGYPFLWALLWVPEDDDDVGNKWLPGFEERIKRENKGIIFRGWAPQMLILNHPAIGGFMTHCGWNAVVEALSFGVPTITLPVFSEQFYTERLISQVLKTGVEVGAEKWTYAFDAGKYPVSREKIATAVKKILDDGEEAEGMRKRAREMKEKAQKSVEEGGSSYNNLTAMIEDLKEFRANNGKAAMKS

SEQ ID NO: 37 - DNA, 1515 bp, Quillaja saponaria
  misc_feature: A nucleic acid molecule comprising a nucleotide which encodes the enzyme according to SEQ ID NO 38.
atggtctccggcgacgacgatgtttctcgtcggccactgaaagtttacttcattgcacacccctcacctggccatattgcccctctgaccaaaatagcccatctcttcgctgccctcggtgagcacgtgactattctcactactcccgccaatgtccacttccatgagaaatccatcgacaaaggaaaggcttccggctatcatgttaacatccacaccgttaaatttccttctaaagaggtcggtctccctgacggcatcgaaaacttctcttacgcctccgatgttgaaacagcagctaaaatttgggctggattcgccatgctacaaactgaaatggagcaatatatggagcttaacccacccgattgcatcgttgccgacatgttcacctcctggacctccgactttgctatcaaattgggaatcacaagaatcgttttcaacgtctattgtattttcacacgctgtttggaagaagccatccgatcaccggactcgccacacttgaacaaagaaatctctgataatgaaccgtttgttatcccgggtctaccagaccccataacaattacccgagctcaactgcccgacggtaccttttctcccatgaaagaactagctagaacagctgagttgaagagctttggaatggtgatcaacgggttttccgaactcgaaaccgattacatcgagcattacaagaaaatcatgggtcacaaacggatttggcatgtcggaccccttcagctaatccaccgtaacgatgaagacaaaattcagaggagccacaagacagcggtgctgagtgataacgataacgagttagtgagttggcttaactcgaagaaacccgactcagttatttacatttgcttcggtagtgcaactcgtttctctaatcaccagctctatgaaatcgcctgtggattagaagcttccgggcacccatttttgtggggcctactttgggtgccagaagatgaagataacgatgacgtgggcaacaaatggttgccagctttcgaagaaagaattaaaaaggaaaataagggaatgattttaagggggtgggctccacagatgttaatcttgaatcacccggcgatcggtggtttcatgacgcattgtggttggaatgcggcggtggaggcgctttcttccggtgttccgattattacatttccggttttctcggatcagttttataatgaaaggctgatatcacaagtgcataagtgtggggtgggggttggtacggaggcgtggagctatgcattcgatgccgggaagaatccggtgggtcgggaaaagataatgacggcggtgaagaagatattagacggtggagaagaggcggaaggaatgagaaagagggcccgggagctgaaagaaatagctaaaagaagtgtggaagaaggtgggtcctcttataataatttaacggctatgattcaagatctgaaagaatttagagctaacaatggcaaggctgcacaagatcatgaatcgtga

SEQ ID NO: 38 - protein, 504 aa, Quillaja saponaria
  misc_feature: An enzyme involved in making QA-TriX from QA-Di (Qs_0283870).
MVSGDDDVSRRPLKVYFIAHPSPGHIAPLTKIAHLFAALGEHVTILTTPANVHFHEKSIDKGKASGYHVNIHTVKFPSKEVGLPDGIENFSYASDVETAAKIWAGFAMLQTEMEQYMELNPPDCIVADMFTSWTSDFAIKLGITRIVFNVYCIFTRCLEEAIRSPDSPHLNKEISDNEPFVIPGLPDPITITRAQLPDGTFSPMKELARTAELKSFGMVINGFSELETDYIEHYKKIMGHKRIWHVGPLQLIHRNDEDKIQRSHKTAVLSDNDNELVSWLNSKKPDSVIYICFGSATRFSNHQLYEIACGLEASGHPFLWGLLWVPEDEDNDDVGNKWLPAFEERIKKENKGMILRGWAPQMLILNHPAIGGFMTHCGWNAAVEALSSGVPIITFPVFSDQFYNERLISQVHKCGVGVGTEAWSYAFDAGKNPVGREKIMTAVKKILDGGEEAEGMRKRARELKEIAKRSVEEGGSSYNNLTAMIQDLKEFRANNGKAAQDHES

SEQ ID NO: 39 - DNA, 1053 bp, Artificial Sequence
  Acanthocystis turfacea chlorella virus 1 UDP-d-glucose 4,6-dehydratase (ATCV-1) coding sequence (1053 bp) NB This sequence was codon-optimised for expression in N. benthamiana. The original sequence can be found as Genbank ID NC_008724.1
atgaatagtcaggagtatactcctaaatcagtccttgtaactggtggggcaggtttcattggcagccatgtcgtgatgaaattggtccagaggtatccggaatgcaaggtcgttgttcttgacaaaatggattactgcgctaccttaaataatcttgctacggtacgagatgcccccaattttaaatttgtaaaaggtgatatacaaagcactgatcttttagctcacgtgctcaaacaggaaaagatagataccattatgcactttgccgcccagacccatgtagataatagctttggcaatagtttagcatttacgatgaacaacgtatacggcactcacgtccttctcgaatgtgcccgattgtatggtggtgttcagagattcattaacgtgtcaactgatgaggtctacggtgagagttccttgggaaagaaggaggggttggacgaacactcctccctcgaaccgacaaatccttatgccgccgcaaaggctggagctgaaatgatggctagagcatatcatacgtcatacaaactcccggtaatagtcacgcgtggcaataatgtatatggtccgcaccagttccccgaaaaaatgatccccaaatttattctcagggcaaccagaggcctcgatttgccaatacatggtgatggcggcgccctgcgatcatacctttacgtcgatgatgttgccgaagcttatattacaattcttttaaagggcaatgttggagagacctataacatcggcacgcaaaaggagcgatccgttgtggacgtggcccatgacatctgcaagatttttaaccgagactcagatacagctatatggcacgttaaagaccgagccttcaatgatcgacgttattttatttctgataaaaaattacttgacctcggctggcaagaaaaaaccacctgggaggacggtcttaagcaaactgtagggtggtatttgcaacatgcaactaggtcctactgggatcacggcaacatggaattagctttagacgcccatccgacacttcaagttcctaaattctaa

SEQ ID NO: 40 - protein, 350 aa, Artificial Sequence [sequence omitted]

SEQ ID NO: 41 - DNA, 693 bp, Artificial Sequence
  Aggregatibacter actinomycetemcomitans NDP-4-keto-6-deoxy-glucose 4-ketoreductase (AaFCD) coding sequence (693 bp) NB This sequence was codon-optimised for expression in N. benthamiana. The original sequence can be found as Genbank ID AB002668.1
atgataattggcaacggtatgctggcaaaagcctttgaatcattccataagcgaacttacaattacataatatttgcatccggagtgagcaactcaaacgaaacttccttcgagaatttcaacagagagaaggaattgcttcttgaagtcctggagcaatataaagacaaaactatcgtttactttagttcctgctccatatacgattctagtttgacgaattctttgtatgtctaccacaaaatgtgtatggagagactggtgcgtgaaaactccaagaattatctcatagcccgtctcccccaagttattggtaaaacgtattcaccaaccattgtcaacttcctttttaacaaaatcaaaaatagggagtgtttcagcatattcggaaaagctcaccgaaattttatcgacgtggatgatgtcgttaaggtcaccaattacttattgaaggagggtctgttcattaacagtattgtgaacttggcaagcacgcaccatacctccatgtacgaattaatcttatatttggaaaaaataagtaatcaacgtgccttctataatgttgagaacaaagggtctaggtactttattgatgtttcaatactgcaggatgtttatcagaagctggggatcaaatttgacaaagattacgtagaaaaggttatcaacaagtactacgctattaagtaa

SEQ ID NO: 42 - protein, 230 aa, Artificial Sequence
  Aggregatibacter actinomycetemcomitans NDP-4-keto-6-deoxy-glucose 4-ketoreductase (AaFCD) translated nucleotide sequence (230 aa)
MIIGNGMLAKAFESFHKRTYNYIIFASGVSNSNETSFENFNREKELLLEVLEQYKDKTIVYFSSCSIYDSSLTNSLYVYHKMCMERLVRENSKNYLIARLPQVIGKTYSPTIVNFLFNKIKNRECFSIFGKAHRNFIDVDDVVKVTNYLLKEGLFINSIVNLASTHHTSMYELILYLEKISNQRAFYNVENKGSRYFIDVSILQDVYQKLGIKFDKDYVEKVINKYYAIK

SEQ ID NO: 43 - DNA, 927 bp, Artificial Sequence
  Anoxybacillus tepidamans NDP-4-keto-6-deoxy-glucose 4-ketoreductase (AtFCD) coding sequence (927 bp) NB This sequence was codon-optimised for expression in N. benthamiana. The original sequence can be found as Genbank ID AY883421.5
atgaagaggatactgatactcggatgcggttacctgggtttaaatctcgcaaactatttttgtaaaaaaaattatgatgtctcagtgatagggagaaagtctgtctatagcaattttttggaagaggagatagagttcatagaagatgatatcaaaaatataaatagttataagcacatgtttaatgaggagacaaccgtcatttacgccataggaagtattaacgcaaataactattttatggacctgaggaatgatatagaaaactcatacatccccttcattaacctccttaactttctttccgaaaagtatattcaaaagttcgtctttctctcttcagccggaacagtctatgggaacgtgaataagaattatataagcgagaatgagattcttaacccaatttcaatctatggtttgcagaaagccttctttgaacaactgataaggattaaaaacaatgaggctagccatttcaggtatttgatcttcagaatatctaacccctatgggggaatcaacattccgaacaagaatcagggaattattccgacgttagtgtacaaagccgtgaacaatgagcctttcgaactttgggcatcaatcaataccatccgtgattatatttacatcgatgaccttagcgaattgatctacaaaacaatctatctggacatttataacgagaccctcaatctcgggtccggtaaaggaacatcaatcaagcaactcattagcctcgtggaggagattttgggaaagaagatcactattcttgaaaagccccccataaagactaacgttttgaaaaatatacttgatatttctaagctcgtcaacaccgtaggctacgaaccaaagatcagcattgaagagggtattagccgttacatcaacactattttaacgaagaacattttttaa

SEQ ID NO: 44 - protein, 308 aa, Artificial Sequence
  Anoxybacillus tepidamans NDP-4-keto-6-deoxy-glucose 4-ketoreductase (AtFCD) translated nucleotide sequence (308 aa)
MKRILILGCGYLGLNLANYFCKKNYDVSVIGRKSVYSNFLEEEIEFIEDDIKNINSYKHMFNEETTVIYAIGSINANNYFMDLRNDIENSYIPFINLLNFLSEKYIQKFVFLSSAGTVYGNVNKNYISENEILNPISIYGLQKAFFEQLIRIKNNEASHFRYLIFRISNPYGGINIPNKNQGIIPTLVYKAVNNEPFELWASINTIRDYIYIDDLSELIYKTIYLDIYNETLNLGSGKGTSIKQLISLVEEILGKKITILEKPPIKTNVLKNILDISKLVNTVGYEPKISIEEGISRYINTILTKNIF

SEQ ID NO: 45 - DNA, 951 bp, Artificial Sequence
  Escherichia coli NDP-4-keto-6-deoxy-glucose 4-ketoreductase (EcFCD) coding sequence (951 bp) NB This sequence was codon-optimised for expression in N. benthamiana. The original sequence can be found as Genbank ID AY528413.1
atggatgctcgtaaaaatggggtattaataaccggtggagctgggttcataggtaaagccttaataactgaaatggtcgaacgtcaaattcccctggtgtcatttgacatcagcgataagcccgacagtttgccagagctttccgaatatttcaactggtataaattctcataccttgagagttcacagaggattaaagagcttcacgaaatagtttccaggcataacatcaaaacggtcatccatttagctacaactatgtttccccacgaatccaaaaagaacatcgataaggattgcttagaaaacgtttatgccaacgtgtgtttctttaagaatttatatgaaaacggctgtgaaaaaattatcttcgcctcatcaggtggcaccgtatatgggaagtctgatacacccttctccgaagacgatgccctgcttcccgaaatttcctacggactgtccaaggttatgactgaaacttatctccgattcatagccaaggaattgaatgggaagtccatctctctcagaatatctaacccctatggtgaggggcaaaggattgacgggaaacaaggagtcattccaattttcctcaataaaatcagcaacgacatccccatcgacatcattggctctatcgaatcaaagcgagactacatttatatttcagatctcgtacaagctttcatgtgctctctggaatatgaaggtcacgaagacatatttaatataggttctggggaaagcataactctgaagaaattgatcgagacgattgagttcaagctgaacaagaaggctgtgattggatttcaagatccgatccacaccaatgccaatggtataattctcgacatcaaacgagccatggcagaactcggctggaggcccaccgtggtcctggatgatggcatcgataaattaatcaagagcattcgatgcaagtaa

SEQ ID NO: 46 - protein, 316 aa, Artificial Sequence
  Echerichia coli NDP-4-keto-6-deoxy-glucose 4-ketoreductase (EcFCD) translated nucleotide sequence (316 aa)
MDARKNGVLITGGAGFIGKALITEMVERQIPLVSFDISDKPDSLPELSEYFNWYKFSYLESSQRIKELHEIVSRHNIKTVIHLATTMFPHESKKNIDKDCLENVYANVCFFKNLYENGCEKIIFASSGGTVYGKSDTPFSEDDALLPEISYGLSKVMTETYLRFIAKELNGKSISLRISNPYGEGQRIDGKQGVIPIFLNKISNDIPIDIIGSIESKRDYIYISDLVQAFMCSLEYEGHEDIFNIGSGESITLKKLIETIEFKLNKKAVIGFQDPIHTNANGIILDIKRAMAELGWRPTVVLDDGIDKLIKSIRCK

SEQ ID NO: 47 - DNA, 897 bp, Quillaja saponaria
  misc_feature: A nucleic acid molecule comprising a nucleotide which encodes the QsFSL-1 enzyme according to SEQ ID NO 48. QsFSL-1
atggcagaagcaacagagaggtatgctgttgtgacaggatctaataaaggaattggatttgggatatgcaagcagctggcttctaaggggattacagtagtgctaacagctagagatgataagagaggtcttgaagcagttgagaaattgaaagaatttgatctgcatggtcatgtgctttttcatcaacttgatgtgtctgatacagctagtgttactagccttgcagattttatcaaaacccagtttgggaaactagatatcttggtaaacaatgcaggtataactggaaccactgtagatgctgatgctttagcatcttcaggctatggtactgggggtgaacgtaaacctattgattggaataaaatagtgatagagacttatgaatcagttgaaaaagctatcaacaccaactattatggagccaaaagaatggctgaagcacttataccccttcttcaagtatcagactcaccaaggattgttaatgcttcctctcctatggcaaagctagagaatattccaagtggatggggtaaggaagtgctaagtgatgttgatagcctaacagaagagaaacttgatgagatgttgacccaattattgaaagatttcaaagagggttcattagaaaccaaaggctggcctactcttatgtcttcgtatataatctcaaaagctgctttaaatgcctacacaaggattcttgctaagaagtacccatctttctgcatcaattgtgtagaccctggtcatgtgaagactgacataaatcgtcacaccggccacttaagtattgatgaaggtgctgaaagccatgtgagattggccctgctgcctgatggtggcccttctggacatttcttctccaggactgaagagacaccattttga

SEQ ID NO: 48 - protein, 298 aa, Quillaja saponaria
  misc_feature: An oxidoreductase enzyme capable of enhancing the activity of a fucosyltransferase (QsFSL-1)
MAEATERYAVVTGSNKGIGFGICKQLASKGITVVLTARDDKRGLEAVEKLKEFDLHGHVLFHQLDVSDTASVTSLADFIKTQFGKLDILVNNAGITGTTVDADALASSGYGTGGERKPIDWNKIVIETYESVEKAINTNYYGAKRMAEALIPLLQVSDSPRIVNASSPMAKLENIPSGWGKEVLSDVDSLTEEKLDEMLTQLLKDFKEGSLETKGWPTLMSSYIISKAALNAYTRILAKKYPSFCINCVDPGHVKTDINRHTGHLSIDEGAESHVRLALLPDGGPSGHFFSRTEETPF

SEQ ID NO: 49 - DNA, 915 bp, Quillaja saponaria
  misc_feature: A nucleic acid molecule comprising a nucleotide which encodes the enzyme according to SEQ ID NO 50. QsFSL-2
atgggttcagatggaagggatgtagcagagaggtatgcagtggttacaggtgcaaacaaaggcataggcctagaaaccgtgcggcaactagcgtctcacggcattacagttgtgttgacagctcgagatgagaagagagggactgaagccacaagaaagctccaccagctgggtttgtcaaatttgattttccatcagctggatgttttagaccctgttagcattcagtcactggccaagttcatccaagacaaatttggcaggcttgatatcctggttaataatgctggagcatctggacttgcagctgatgagaaagctctgaaggcattaaacatagataatgcagcttggctctcaggcaaggccgccaatttagttcaaggaattgtcacacatacctatgagcaaggcgaagaatgcataaatacaaactattatggtgtcaaaagggtgacggaagctctcctaccgctgttacaactttcccctataggagcaaggataataaatgtttcctcttgcaggggtgagctaaagaggattccaatgaacgtaagaaatgaactgggcgacatcaaagttctgactgaaggcagaatagatgcaattttgatgaaatttctacacgattttaaggataatgcacttgagtccaacggatggacattgatggggcctgcttatagcatttcgaaggccagtctcaatgcctacactagacttcttgccaaaaagtaccccgagatgctcattaactgtgttcatcctggttatgtcaacacagatatgacttggcatagagggatactgacggtagaagagggtgctaaaggcccagccatgctagctcttttgcaagatggaggacctacaggttgctattttgatagtactcaacaggcagaattttaa

SEQ ID NO: 50 - protein, 304 aa, Quillaja saponaria
  misc_feature: An oxidoreductase enzyme capable of enhancing the activity of a fucosyltransferase (QsFSL-2)
MGSDGRDVAERYAVVTGANKGIGLETVRQLASHGITVVLTARDEKRGTEATRKLHQLGLSNLIFHQLDVLDPVSIQSLAKFIQDKFGRLDILVNNAGASGLAADEKALKALNIDNAAWLSGKAANLVQGIVTHTYEQGEECINTNYYGVKRVTEALLPLLQLSPIGARIINVSSCRGELKRIPMNVRNELGDIKVLTEGRIDAILMKFLHDFKDNALESNGWTLMGPAYSISKASLNAYTRLLAKKYPEMLINCVHPGYVNTDMTWHRGILTVEEGAKGPAMLALLQDGGPTGCYFDSTQQAEF

SEQ ID NO: 51 - DNA, 909 bp, Salvia officinalis
  misc_feature: A nucleic acid molecule comprising a nucleotide which encodes the enzyme according to SEQ ID NO 52. SoFSL-1
atggctgaagcatcctcatttcttgcacagaaaaggtatgcggtcgtgacaggagcaaacaaaggactaggactagaaatatgcggacagcttgcttcacagggggtgacggtactgctgacatccagagatgaaaaacgaggcttagaagccattgaggagcttaagaaatcggggattaattcggaaaatcttgaatatcatcagctggatgttactaagccagctagtttcgcttctctggccgatttcatcaaggccaaatttggcaagcttgatatcctggtgaacaatgcagggatcagcggtgttattgtagattatgcagctttaatggaagccattcgccgtcgaggggcagagatcaattacgatggagtgatgaaacagacctacgagctagcagaggaatgcttgcaaacaaattactatggtgtgaaaagaaccattaatgctctccttccgctacttcagttttccgattcaccaaggatcgtcaatgtttcctccgatgttggcctccttaagaaaatacccggcgagagaatcagagaagccttaggcgacgtggaaaaacttacggaagaaagcgtggacgggattttagacgagtttctaagagatttcaaggaaggcaagatcgcagagaaaggttggcctacgtttaagagcgcctattcaatctcaaaggcggcgctcaattcgtacacgagggttttagcacggaaatacccgtcgatcatcatcaactgtgtctgcccgggtgtcgtcaaaaccgatatcaatcttaaaatgggccacttgacggttgaagaaggcgcggccagtcccgtgaggttagcactcatgccccttggttcgccttccggcctgttctatactcgaaacgaagtaactccatttgaatga

SEQ ID NO: 52 - protein, 302 aa, Salvia officinalis
  misc_feature: An oxidoreductase enzyme capable of enhancing the activity of a fucosyltransferase (SoFSL-1)
MAEASSFLAQKRYAVVTGANKGLGLEICGQLASQGVTVLLTSRDEKRGLEAIEELKKSGINSENLEYHQLDVTKPASFASLADFIKAKFGKLDILVNNAGISGVIVDYAALMEAIRRRGAEINYDGVMKQTYELAEECLQTNYYGVKRTINALLPLLQFSDSPRIVNVSSDVGLLKKIPGERIREALGDVEKLTEESVDGILDEFLRDFKEGKIAEKGWPTFKSAYSISKAALNSYTRVLARKYPSIIINCVCPGVVKTDINLKMGHLTVEEGAASPVRLALMPLGSPSGLFYTRNEVTPFE

SEQ ID NO: 53 - DNA, 900 bp, Spinacia oleracea
  misc_feature: A nucleic acid molecule comprising a nucleotide which encodes the enzyme according to SEQ ID NO 54. SpolFSL
atggctgaacaatccaactttctggctgaaaaaaggtatgcagtagtgacaggtgcaaacaaaggaatagggcttgaaatatgcagacagcttgcttctcaaggtgtgattgtacttatcacttctagagatggaaagaaaggattagaagcccttaatgatctcattaaatctggaattagctctgataatcttcattatcatcagcttgatgttactgaccctatgagtattactgctcttgctggtttcatcaattccaaatttggcaagcttgatattctggtgaacaatgctgggataggtggatttataattgactacgatgctatcaaagcaataggttttcgcaatatcaattatgacgagatgatgacacaaacatatgagcttgcaaaagaatgcttggaaacaaactactatggagttaagagaacaactgaagctttgcttcctcagctggagttatcggattcaccaaggatcatcaatgtctcctcttctacggggatgttgaagaatataccaaatgagaggatcagaggagtcttgggtgatgcagagaatcttacagaagaaaaagttgaagcgattttgaatgagttactgacagatttcaaagatggttcattcaaagagaaagaatggccttctagaatggcagcttatacactgtcaaaggcggctttgaatgcatatgcaagaatattggctaagaaatacccgtcaattatcatcagttgtgtttgtcctggtgttactaagacagatatgaacggaaacttgggacaattaacagttgaagaaggggccgcaagtccggtgagagtagcattgatgcctcatggttcaccttccggtcttttctatgcaagaagcgaagtttcttcatatgaataa

SEQ ID NO: 54 - protein, 299 aa, Spinacia oleracea
  misc_feature: An oxidoreductase enzyme capable of enhancing the activity of a fucosyltransferase (SpolFSL).
MAEQSNFLAEKRYAVVTGANKGIGLEICRQLASQGVIVLITSRDGKKGLEALNDLIKSGISSDNLHYHQLDVTDPMSITALAGFINSKFGKLDILVNNAGIGGFIIDYDAIKAIGFRNINYDEMMTQTYELAKECLETNYYGVKRTTEALLPQLELSDSPRIINVSSSTGMLKNIPNERIRGVLGDAENLTEEKVEAILNELLTDFKDGSFKEKEWPSRMAAYTLSKAALNAYARILAKKYPSIIISCVCPGVTKTDMNGNLGQLTVEEGAASPVRVALMPHGSPSGLFYARSEVSSYE

(19) United States
(12) Patent Application Publication
(10) Pub. No.: US 2024/0102069 A1
(43) Pub. Date: Mar. 28, 2024

(54) METHODS AND COMPOSITIONS

(71) Applicant: PLANT BIOSCIENCE LIMITED, Norwich (GB)

(72) Inventors: Anne OSBOURN, Norwich (GB); James REED, Norwich (GB); Anastasia ORME, Norwich (GB)

(21) Appl. No.: 18/258,931

(22) PCT Filed: Dec. 22, 2021
(86) PCT No.: PCT/EP2021/087323

§ 371 Date: Jun. 22, 2023

(30) Foreign Application Priority Data
Dec. 24, 2020 (GB) 2020623.1
Nov. 17, 2021 (GB) 2116554.3

(51) Int. Cl. C12P 19/56; C12N 15/82

(52) U.S. Cl. CPC C12P 19/56; C12N 15/8205; C12N 15/8257

(57) ABSTRACT

The present invention relates to a biosynthetic route to intermediates of the QS-21 molecule which are triterpenes from a saponin extract of the tree Quillaja saponaria comprising a quillaic acid backbone, as well as routes to make the QS-21 molecule, enzymes involved, the products produced and uses of the product.

The present invention relates to a biosynthetic route to intermediates of the QS-21 molecule, as well as routes to make the QS-21 molecule, enzymes involved, the products produced and uses of the product.

### BACKGROUND

QS-21 is a natural saponin extract from the bark of the Chilean ‘soapbark’ tree, Quillaja saponaria. QS-21 extract was originally identified as a purified fraction of a crude bark extract of Quillaja Saponaria Molina obtained by RP-HPLC purification (peak 21) (Kensil et al. 1991). QS-21 extract, or fraction, comprises several distinct saponin molecules. Two principal isomeric molecular constituents of the fraction were reported (Ragupathi et al. 2011) and are depicted in FIG. 1. Both incorporate a central triterpene core, to which a branched trisaccharide is attached at the terpene C-3 oxygen functionality, and a linear tetrasaccharide is linked to the triterpene C-28 carboxylate group. A fourth component within the saponin structure is a glycosylated pseudo-dimeric acyl chain attached to the fucose moiety via a hydrolytically labile ester linkage. The isomeric components differ in the constitution of the terminal sugar residue of the tetrasaccharide, in which the major and minor compounds incorporate either an apiose (65%) or a xylose (35%) carbohydrate, respectively.

Saponins from Q. saponaria, including QS-21 have been known for many years to have potent immunostimulatory properties, capable of enhancing antibody production and specific T-cell responses. These properties have resulted in the development of Quillaja saponin-based adjuvants for vaccines. Of particular note, the ASO1 adjuvant features a liposomal formulation of QS-21 and 3-O-desacyl-4′-monophosphoryl lipid A (the production of which is described in WO2013/041572) and is currently licenced in vaccine formulations for diseases including shingles (Shingrix) and malaria (Mosquirix).

The present invention describes methods to synthesise intermediates of the QS-21 molecule as well as the QS-21 molecule other than by purification from the native Q. saponaria plant and the resulting product, which is useful as an adjuvant in vaccine formulations. The present invention also relates to enzymes involved in the methods, vectors, host cells and biological systems to produce the product.

### BRIEF DESCRIPTION OF THE INVENTION

The present invention relates, in particular, to the biosynthetic addition of the C-28 linear tetrasaccharide to a molecule comprising a quillaic acid backbone (QA) and the resulting QA derivative. The invention includes the biosynthetic preparation of intermediates of the QS-21 molecule, such as, for example, QA-FRX(X/A) or QA-Tri(X/R)-FRX(X/A), as well as chemical routes to make the QS-21 molecule, all component parts to make the derivatives and molecules, as well as uses thereof.

QA biosynthesis derives from the simple triterpene β-amyrin, which is synthesised through cyclisation of the universal linear precursor 2,3-oxidosqualene (OS) by an oxidosqualene cyclase (OSC). This biosynthesis is known in the art, such as WO2019/122259, the content of which is incorporated by reference. This β-amyrin scaffold is further oxidised with a carboxylic acid, alcohol and aldehyde at the C-28, C-16a and C-23 positions, respectively, by a series of three cytochrome P450 monooxygenases, forming quillaic acid (QA). The OSC and C-28, C16α and C-23 oxidases are referred to herein as QsbAS (β-amyrin synthase), QsCYP716-C-28, QsCYP716-C-16a and QsCYP714-C-23 oxidases, respectively. A biosynthetic pathway for this is given in FIG. 2.

The branched trisaccharide chain in QS-21 is initiated with a D-glucopyranuronic acid (D-GlcpA) residue attached with a β-linkage at the C-3 position of the QA backbone. The D-GlcpA residue has two sugars linked to it: a D-galactopyranose (D-Galp) attached with a β-1,2-linkage and either a D-xylopyranose (D-Xylp) or an L-rhamnopyranose (L-Rhap) attached with a β-1,3-linkage or an α-1,3-linkage, respectively. A schematic for the glycosylation of QA to 3-O-{α-L-rhamnopyranosyl-(1->3)-[3-D-galactopyranosyl-(1->2)]-β-D-glucopyranosiduronic acid}-quillaic acid (QA-TriR) or 3-O-{β-D-xylopyranosyl-(1->3)-[β-D-galactopyranosyl-(1->2)]-β-D-glucopyranosiduronic acid}-quillaic acid (QA-TriX) is shown in FIG. 3. Seven enzymes have been identified that have activity relevant to the production of the QA 3-0 trisaccharide, such as in PCT/EP2020/067866 (published as WO2020/260475). These include two functionally-redundant glucuronosyltransferases, CSL1 and CslG2, that can add the initial β-D-glucopyranuronic acid at the C-3 position of quillaic acid; a galactosyltransferase, Qs-3-O-GalT, that adds the β-D-galactopyranose to the C-2 position of the β-D-glucopyranuronic acid; a xylosyltransferase, Qs_0283870, that adds the β-D-xylopyranose at the C-3 position of the β-D-glucopyranuronic acid; two rhamnosyltransferases, DN20529_c0_g2_i8 and Qs_0283850, that add an α-L-rhamnopyranose at the C-3 position of the β-D-glucopyranuronic acid; and a bifunctional enzyme, Qs-3-O-RhaT/XylT that can add either a β-D-xylopyranose or a α-L-rhamnopyranose to the C-3 position of the β-D-glucopyranuronic acid (FIG. 3). For simplicity, throughout the application, a QA derivative including the branched trisaccharide at position C-3 may be designated as “QA-TriX”, “QA-TriR” or “QA-Tri(X/R)”.

The present invention describes, for the first time, the biosynthetic route of the addition of the linear tetrasaccharide at the C-28 position of the QA backbone and the resulting derivatives, such as, for example, QA-FRX(X/A) or QA-Tri(X/R)-FRX(X/A), including those to chemically produce the QS-21 molecule, other than by purification from the native Q. saponaria plant.

Accordingly, the present invention provides methods for making QA derivatives, QA derivatives obtainable therefrom, enzymes used in the methods, nucleic acids encoding the enzymes, vectors comprising the nucleic acids, host cells transformed with the vectors.

### DESCRIPTION OF THE FIGURES

FIG. 1 shows the structure of QS-21. The core backbone is formed from the triterpene quillaic acid (QA). The C-3 position features a branched trisaccharide consisting of β-D-glucopyranuronic acid (D-GlcpA), β-D-galactopyranose (D-Galp) and either a β-D-xylopyranose (D-xylp) or α-L-rhamnopyranose (L-rhap) at (R₁). The C-28 position features a linear tetrasaccharide consisting of β-D-fucopyranose (D-fucp), α-L-rhamnopyranose, β-D-xylopyranose and either a terminal β-D-apiofuranose (D-apif) or β-D-xylopyranose at (R₂). The D-fucose also features an 18-carbon acyl chain which terminates with α-L-arabinofuranose (L-Araf). Carbon numbering is indicated in FIG. 2.

FIG. 2 shows the production of quillaic acid (QA) from 2,3-oxidosqualene via β-amyrin. Numbering of important β-amyrin carbons referred to herein are labelled in FIG. 2. The pathway from β-amyrin requires oxidation at three (C-28, C-23 and C-16a) positions. These oxidation steps are shown in a linear fashion for simplicity; however, they could occur in any order.

FIG. 3 shows the production of QA-TriR or QA-TriX from quillaic acid (QA). A β-D-glucopyranuronic acid (β-D-GlcpA) is added, by either of the glucuronosyltransferases QsCLS1 or QsCsIG2, to the C-3 position of quillaic acid to form QA-Mono. The galactosyltransferase Qs-3-O-GalT adds a β-D-galactopyranose (β-D-Galp) to the C-2 position of the glucopyranuronic acid to form QA-Di. An α-L-rhamnopyranose (α-L-Rhap) can be attached to the C-3 position of the glucopyranuronic acid by the single-function rhamnosyltransferases, DN20529_c0_g2_i8 or Qs_0283850, or by the dual-function Qs-3-O-RhaT/XylT, to form QA-TriR. Alternatively, a β-D-xylopyranose (β-D-Xylp) can be attached to the C-3 position of the glucopyranuronic acid to form QA-TriX, either by the single-function xylosyltransferase Qs_0283870 or by the dual-function Qs-3-O-RhaT/XylT.

FIG. 4 shows the proposed biosynthesis of the QS-21 C-28 linear tetrasaccharide chain from QA-Tri(X/R). The chain is initiated with a β-D-fucopyranose (β-D-Fucp) attached to the C-28 of quillaic acid via an ester linkage, followed by the attachment of an α-1,2-L-rhamnopyranose (α-L-Rhap) and the attachment of a β-1,4-D-xylopyranose (β-D-Xylp). The terminal sugar of the chain can be either β-1,3-D-xylopyranose (β-D-Xylp) or β-1,3-D-apiofuranose (β-D-Apif). For simplicity, the resulting QA derivative may be designated as “QA-Tri (X/R)-FRX(X/A)”.

FIG. 5 shows the identification of a triterpene C-28 fucosyltransferase (Qs-28-O-FucT). Leaf extracts from N. benthamiana transiently expressing Q. saponaria genes were analysed by HPLC-CAD-MS. HPLC-CAD traces (top) and extracted ion chromatograms (EICs) (bottom) are shown. Co-expression of the genes required for the production of QA-Tri(X/R) (AstHMGR+QsbAS+QsCYP716-C-28+QsCYP716-C-16α+QsCYP714-C-23+QsCSL1+Qs-3-O-GalT+Qs-3-O-RhaT/XylT) yielded two overlapping peaks of QA-TriR (12.6 minutes, m/z=969) and QA-TriX (12.8 minutes, m/z=955). Further co-expression of QsUGT_L2 resulted in the accumulation of new more polar peaks between 11.6 and 12.3 minutes, which have mass ions (m/z=1115 and 1101) consistent with the addition of a pentose to QA-TriR and QA-TriX to form QA-TriR-F (MW=1116.54) and QA-TriX-F (MW=1102.52), respectively. IS, internal standard (digitoxin).

FIG. 6 shows the identification of a triterpene C-28 rhamnosyltransferase (Qs-28-O-RhaT). Leaf extracts from N. benthamiana transiently expressing Q. saponaria genes were analysed by HPLC-CAD-MS. HPLC-CAD traces (top) and extracted ion chromatograms (EICs) (bottom) are shown. Co-expression of the genes required for the production of QA-TriX (AstHMGR+QsbAS+QsCYP716-C-28+QsCYP716-C-16α+QsCYP714-C-23+QsCSL1+Qs-3-O-GalT+Qs_0283870) with Qs-28-O-FucT (i.e. QsUGT_L2) yielded a peak of QA-TriX (12.8 minutes, m/z=955) and a peak of QA-TriX-F (12.0 minutes, m/z=1101). Further co-expression of QsUGT_A6 resulted in the reduction of the QA-TriX-F peak and the accumulation of a more polar peak at 11.6 minutes with a mass ion (m/z=1247) consistent with the addition of a rhamnose to QA-TriX-F to form QA-TriX-FR (MW=1248.58). Further co-expression of QsUGT_A6 without co-expressing Qs-28-O-FucT (i.e. QsUGT_L2) resulted in the accumulation of the precursor QA-TriX only. IS, internal standard (digitoxin).

FIG. 7 shows the identification of a triterpene C-28 xylosyltransferase (Qs-28-O-XylT3). Leaf extracts from N. benthamiana transiently expressing Q. saponaria genes were analysed by HPLC-CAD-MS. HPLC-CAD traces (top) and selected extracted ion chromatograms (EICs) (bottom) are shown. Co-expression of the genes required for the production of QA-TriX-F (AstHMGR+QsbAS+QsCYP716-C-28+QsCYP716-C-16α+QsCYP714-C-23+QsCSL1+Qs-3-O-GalT+Qs_0283870+Qs-28-O-FucT [i.e. QsUGT_L2]) yielded a peak of QA-TriX (12.8 minutes, m/z=955) and a peak of QA-TriX-F (12.0 minutes, m/z=1101). Further co-expression of Qs-28-O-RhaT (i.e. QsUGT_A6) resulted in the accumulation of QA-TriX-FR (11.6 minutes). Further co-expression of QsUGT_A7 resulted in the reduction of the QA-TriX-FR and QA-TriX peaks and the accumulation of a peak at 11.9 minutes with a mass (m/z=1379) consistent with the addition of a xylose to QA-TriX-FR to form QA-TriX-FRX (MW=1380.62). Further co-expression of QsUGT_A7 without Qs-28-O-RhaT (i.e. QsUGT_A6) resulted in the accumulation of QA-TriX and QA-TriX-F. IS, internal standard (digitoxin).

FIG. 8 shows the identification of a triterpene C-28 glucosyltransferase. Leaf extracts from N. benthamiana transiently expressing Q. saponaria and Centella asiatica genes were analysed by HPLC-CAD-MS. HPLC-CAD traces (top) and proposed pathway (bottom) are shown. Co-expression of the genes required for the production of QA-TriX (AstHMGR+QsbAS+QsCYP716-C-28+QsCYP716-C-16α+QsCYP714-C-23+QsCsIG2+Qs-3-O-GalT+Qs_0283870) yielded a peak of QA-TriX (12.8 minutes, m/z=955). Further co-expression of the C-28 glucosyltransferase CaUGT73AD1 (de Costa et al, 2017) resulted in the accumulation of a peak at 10.1 minutes (m/z=1117) with a mass consistent with the addition of glucose (Glcp) to QA-TriX to form QA-TriX-G, and an additional new peak at 11.8 minutes (m/z=1101) consistent with the addition of glucose to Gyp-TriX, an intermediate lacking the C-16 oxidation by QsCYP716-C-16α. Further co-expression of Qs-28-O-RhaT resulted in the reduction of the QA-TriX-G and Gyp-TriX-G peaks and the accumulation of two more polar peaks at 9.5 minutes (m/z=1263) and 11.1 minutes (m/z=1247) with masses consistent with the addition of rhamnose (Rhap) to QA-TriX-G and Gyp-TriX-G to form QA-TriX-GR (MW=1264.57) and Gyp-TriX-GR (MW=1248.58), respectively. Further co-expression of Qs-28-O-XylT3 resulted in the reduction of the peaks at 9.5 minutes and 11.1 minutes and the accumulation of two new peaks at 9.8 minutes (m/z=1395) and 11.5 minutes (m/z=1379) which have mass ions consistent with the addition of a xylose (Xylp) to QA-TriX-GR and Gyp-TriX-GR to form QA-TriX-GRX (MW=1396.61) and Gyp-TriX-GRX (MW=1380.62), respectively. IS, internal standard (digitoxin).

FIG. 9 shows the identification of a triterpene C-28 xylosyl/apiosyltransferases. Leaf extracts from N. benthamiana transiently expressing Q. saponaria and Centella asiatica genes were analysed by HPLC-CAD-MS. Co-expression of the genes required for the production of QA-TriX-GRX (AstHMGR+QsbAS+QsCYP716-C-28+QsCYP716-C-16a+QsCYP714-C-23+QsCsIG2+Qs-3-O-GalT+Qs_0283870+CaUGT73AD1+Qs-28-O-RhaT+Qs-28-O-XylT3) yielded a peak of QA-TriX (12.8 minutes, m/z=955), a peak of QA-TriX-GRX (9.5 minutes, m/z=1395) and a peak of Gyp-TriX-GRX (11.2 minutes, m/z=1379). Further co-expression of QsAXS1 did not alter the accumulation of these peaks. Further co-expression of QsAXS1 with QsUGT_D3 resulted in the reduction of the QA-TriX-GRX and Gyp-TriX-GRX peaks and the accumulation of peaks at 9.6 minutes (m/z=1528) and 11.5 minutes (m/z=1512). Further co-expression of QsAXS1 with the two candidates QsUGT_D2 and QsUGT_A3 also resulted in a reduction of the QA-TriX-GRX and Gyp-TriX-GRX peaks and the accumulation of peaks at 9.7 minutes (m/z=1528) and 11.6 minutes (m/z=1512). IS, internal standard (digitoxin).

FIG. 10 shows that the activity of QsUGT_D2 is dependent on QsAXS1. Leaf extracts from N. benthamiana transiently expressing Q. saponaria and Centella asiatica genes were analysed by HPLC-CAD-MS. Extracted ion chromatograms are shown. Co-expression of the genes required for the production of QA-TriX-GRX (AstHMGR+QsbAS+QsCYP716-C-28+QsCYP716-C-16α+QsCYP714-C-23+QsCsIG2+Qs-3-O-GalT+Qs_0283870+CaUGT73AD1+Qs-28-O-RhaT+Qs-28-O-XylT3) with QsUGT_D3 alone resulted in the accumulation of a peak of QA-TriX at 12.8 minutes (m/z=955) and two peaks at 9.6 minutes (m/z=1528) and 11.5 minutes (m/z=1512) with mass ions consistent with QA-TriX-GRX(X/A) (MW=1528.66) and Gyp-TriX-GRX(X/A) (MW=1512.66), respectively. Co-expression of QsUGT_D2 alone with the genes required to produce QA-TriX-GRX resulted in peaks of QA-TriX at 12.8 minutes (m/z=955), QA-TriX-GRX (9.5 minutes, m/z=1395), Gyp-TriX-GRX (11.2 minutes, m/z=1379 and only trace accumulation of peaks at 9.7 minutes (m/z=1528) and 11.6 minutes (m/z=1512). The addition of QsUGT_D2 along with QsAXS1 resulted in a larger reduction of the QA-TriX-GRX and Gyp-TriX-GRX peaks and the increased accumulation of the peaks at 9.7 minutes (m/z=1528) and 11.6 minutes (m/z=1512). These latter peaks are consistent with the accumulation of QA-TriX-GRX(X/A) (MW=1528.66) and Gyp-TriX-GRX(X/A) (MW=1512.66), respectively. IS, internal standard (digitoxin).

FIG. 11 shows the production of UDP-α-D-fucose in N. benthamiana. Sugar nucleotide analysis of N. benthamiana plants was performed, with the traces for UDP-deoxy-hexoses shown. Control plants (top) infiltrated with water show only a single peak, confirmed as UDP-R-L-rhamnose against an authentic standard (standard not shown). Plants infiltrated with a 50 mM solution of D-fucose (middle) show accumulation of two new peaks (labelled 1 and 2). Peak 1 was shown to be UDP-α-D-fucose by spiking the sample with an authentic standard of UDP-α-D-fucose (bottom). The second peak (2) is believed to be UDP-α-D-quinovose, resulting from C-4 epimerisation of UDP-α-D-fucose by endogenous epimerase enzymes (such as UDP-D-glucose/UDP-D-galactose 4-epimerase).

FIG. 12 shows that infiltrating D-fucose enhances production of the D-fucosylated QA derivatives. The enzymes necessary for production of the QA-TriX-F (AstHMGR+QsbAS+QsCYP716-C-28+QsCYP716-C-16α+QsCYP714-C-23+QsCSL1+Qs-3-O-GalT+Qs_0283870+Qs-28-O-FucT) were transiently co-expressed in N. benthamiana either alone (top) or with the addition of 50 mM D-fucose in the infiltration buffer (bottom). Analysis of the extracts by LC-MS revealed the presence of D-fucose was enough to enhance the production of QA-TriX-F by several fold. Results are presented as extract ion chromatograms for QA-TriX-F (m/z 1101, black) and the internal standard digitoxin (formate adduct=809, grey).

FIG. 13 shows the biosynthesis of NDP-D-fucose from NDP-D-glucose.

FIG. 14 shows that the transient expression of NDP-D-fucose biosynthetic enzymes can boost the levels of the fucosylated products N. benthamiana. Transient expression of the enzymes for production of the QA-TriX-F compound in N. benthamiana was performed (AstHMGR, QsbAS, QsCYP716-C-28+QsCYP716-C-16α+QsCYP714-C-23+QsCsIG2+Qs-3-O-GalT+Qs_0283870+Qs-28-O-FucT). In addition, a series of enzymes involved in NDP-D-fucose biosynthesis from various non-plant species were transiently co-expressed with the above enzymes to determine their ability to boost the yield of the QA-TriX-F product. These included either the Acanthocystis turfacea chlorella virus 1 UDP-D-glucose 4,6-dehydratase (ATCV-1), or three bacterial 4-ketoreductase (FCD) enzymes from Aggregatibacter actinomycetemcomitans (AaFCD), Anoxybacillus tepidamans (AtFCD), or Escherichia coli (EcFCD). Control samples were also performed including addition of 50 mM D-fucose (positive control), or without fucose-boosting (QA-TriX-F enzymes only). Leaf extracts were analysed by LC-MS/CAD and results are shown as A) CAD chromatograms or B) MS extract ion chromatograms (EIC). EIC masses were selected for the QS-TriX-F product (m/z 1101) and the internal standard digitoxin (formate adduct=809). In any of the samples expressing an NDP-D-fucose biosynthetic enzyme (either ATCV-1 or the FCD enzymes), a clear increase to the QA-TriX-F product could be seen compared to the non-boosted control. The amount of product was similar to that found in the positive control (+50 mM D-fucose).

FIG. 15 shows that the co-expression of ATCV-1 and AaFCD has little effect on QA-TriX-F yields compared to expression of either enzyme individually. Transient expression of the enzymes for production of QA-TriX-F in N. benthamiana was performed (AstHMGR, QsbAS, QsCYP716-C-28+QsCYP716-C-16α+QsCYP714-C-23+QsCsIG2+Qs-3-O-GalT+Qs_0283870+Qs-28-O-FucT). In addition, the Acanthocystis turfacea chlorella virus 1 UDP-D-glucose 4,6-dehydratase (ATCV-1), or 4-ketoreductase (FCD) from Aggregatibacter actinomycetemcomitans (AaFCD) were also co-expressed, either individually, or together. Leaf extracts were analysed by LC-MS and results are shown as MS extract ion chromatograms (EIC). EIC masses were selected for the QS-TriX-F product (m/z 1101) and the internal standard digitoxin (formate adduct=809). Co-expression of both ATCV-1 and AaFCD made little difference compared to expression of either enzyme individually.

FIG. 16 shows enhancing production of the fucosylated compounds by transient co-expression of the Q. saponaria oxidoreductase (FucSyn). Top—LC-CAD traces of N. benthamiana leaf extracts following transient expression of the enzymes necessary for the QA-TriR product (AstHMGR+QsbAS+QsCYP716-C-28+QsCYP716-C-16α+QsCYP714-C-23+QsCsIG2+Qs-3-O-GalT+Qs_0283850—top chromatogram), or the QA-TriR-F product (AstHMGR+QsbAS+QsCYP716-C-28+QsCYP716-C-16α+QsCYP714-C-23+QsCsIG2+Qs-3-O-GalT+Qs_0283850+Qs-28-O-FucT—second chromatogram). When the clustered oxidoreductase (FucSyn) was co-expressed with the QA-TriR-F gene set, a large increase in product at 11.5 mins was observed (bottom chromatogram). When the oxidoreductase was expressed in the absence of the fucosyltransferase Qs-28-O-FucT (third chromatogram), the product was no longer observed, demonstrating that both the oxidoreductase and fucosyltransferase are necessary for high level production of the product at 11.5 min. Bottom—The mass spectrum of this compound at 11.5 min gave a prominent ion at m/z 1115. This is consistent with the predicted molecular weight of the QA-TriR-F (MW=1116.54).

FIG. 17 shows a comparison of the efficacy of different boosting strategies described herein. The gene set necessary for production of the QA-TriR-F was transiently co-expressed in N. benthamiana (AstHMGR+QsbAS+QsCYP716-C-28+QsCYP716-C-16α+QsCYP714-C-23+QsCsIG2+Qs-3-O-GalT+Qs_0283850+Qs-28-O-FucT). To compare relative amounts of the QA-TriR-F, the QA-TriR-F enzyme sets were co-infiltrated with either 50 mM D-fucose, the Acanthocystis turfacea chlorella virus 1 UDP-D-glucose 4,6-dehydratase (ATCV-1) or the QsFucSyn enzyme. Results are presented as LC-CAD data normalised to the internal standard (digitoxin, 16 mins). The QA-TriR-F is seen at 11.5 min and shows highest accumulation in the QsFucSyn-expressing samples.

FIG. 18 shows building the C-28 glycoside and boosting yields with QsFucSyn. The C-28 tetrasaccharide chain of the QA-TriR molecule was built step-by-step from QA-Tri-FR to QA-Tri-FRXA by transient expression of the relevant gene sets (QA-TriR-FR—top (AstHMGR+QsbAS+QsCYP716-C-28+QsCYP716-C-16α+QsCYP714-C-23+QsCsIG2+Qs-3-O-GalT+Qs_0283850+Qs-28-O-FucT+Qs-28-O-RhaT—top); QA-TriR-FRX—middle (AstHMGR+QsbAS+QsCYP716-C-28+QsCYP716-C-16α+QsCYP714-C-23+QsCsIG2+Qs-3-O-GalT+Qs_0283850+Qs-28-O-FucT+Qs-28-O-RhaT+Qs-28-O-XylT3) and QA-TriR-FRXA—bottom (AstHMGR+QsbAS+QsCYP716-C-28+QsCYP716-C-16α+QsCYP714-C-23+QsCsIG2+Qs-3-O-GalT+Qs_0283850+Qs-28-O-FucT+Qs-28-O-RhaT+Qs-28-O-XylT3+Qs-28-O-ApiT4+QsAXS1). Each of these enzyme sets were tested either in the presence or absence of the QsFucSyn enzyme. Results are presented as LC-CAD traces (left). In each case, a visible increase in the relevant product was observed in the presence of QsFucSyn. The accompanying mass spectra (right) of the products corresponded with the mass of the expected products.

FIG. 19 shows the production of the full C-28 tetrasaccharide chain with differing terminal sugar variants. The set of enzymes necessary for production of QA-TriR-FRX (AstHMGR+QsbAS+QsCYP716-C-28+QsCYP716-C-16α+QsCYP714-C-23+QsCsIG2+Qs-3-O-GalT+Qs_0283850+Qs-28-O-FucT+Qs-28-O-RhaT+Qs-28-O-XylT3—top), QA-TriR-FRXX (AstHMGR+QsbAS+QsCYP716-C-28+QsCYP716-C-16α+QsCYP714-C-23+QsCsIG2+Qs-3-O-GalT+Qs_0283850+Qs-28-O-FucT+Qs-28-O-RhaT+Qs-28-O-XylT3+Qs-28-O-XylT4—middle) and QA-TriR-FRXA (AstHMGR+QsbAS+QsCYP716-C-28+QsCYP716-C-16α+QsCYP714-C-23+QsCsIG2+Qs-3-O-GalT+Qs_0283850+Qs-28-O-FucT+Qs-28-O-RhaT+Qs-28-O-XylT3+Qs-28-O-ApiT4—bottom) were expressed in the presence of the QsFucSyn enzyme. A peak with a mass ion (m/z=1526) corresponding to the fully glycosylated products QA-TriR-FRXX or QA-TriR-FRXA (MW=1526.68) could be detected only when the enzymes for the full sugar chains were expressed (middle and bottom), but not the in control (top). NB: in this experiment the UDP-apiose/UDP-xylose synthase (QsAXS1) was not included.

FIG. 20 demonstrates the importance of QsAXS1 for efficient apiosylation of the C-28 tetrasaccharide chain. The set of enzymes necessary for production of QA-TriR-FRX (AstHMGR+QsbAS+QsCYP716-C-28+QsCYP716-C-16α+QsCYP714-C-23+QsCsIG2+Qs-3-O-GalT+Qs_0283850+Qs-28-O-FucT+Qs-28-O-RhaT+Qs-28-O-XylT3+QsFucSyn (FucSyn)—top) were expressed. The set of enzymes necessary for the production of QA-TriR-FRXA (AstHMGR+QsbAS+QsCYP716-C-28+QsCYP716-C-16α+QsCYP714-C-23+QsCsIG2+Qs-3-O-GalT+Qs_0283850+Qs-28-O-FucT+Qs-28-O-RhaT+Qs-28-O-XylT3+Qs-28-O-ApiT4+QsFucSyn) were expressed in the absence (middle) or presence (bottom) of QsAXS1 (AXS). Extract ion chromatograms (EICs) for the molecular weight of the QA-TriR-FRXA product (MW=1526.68) are shown. In control plants lacking the apiosyltransferase Qs-28-O-ApiT (top), no signal is present for the QA-TriR-FRXA product. Upon expression of the apiosyltransferase Qs-28-O-ApiT (middle), a small signal at 11.6 minutes is visible. Analysis of the mass spectrum at this time point reveals the major ion to be 1394, corresponding to the QA-TriR-FRX product as seen in controls, suggesting poor conversion of this product to QA-TriR-FRXA. Finally, co-expression of the QsAXS1 enzyme with the QA-TriR-FRXA enzymes resulted in a large increase in the 1526 ion (bottom). Accordingly, this was the most abundant product in the mass spectra at 11.6 mins (NB: this is visible predominantly as an ion at 1527 due to increased incorporation of ¹³C).

FIG. 21 shows a comparison of the impact of co-expression of QsFucSyn and ATCV-1 on QA-TriR-F yields. The gene set necessary for production of QA-TriR-F was transiently co-expressed in N. benthamiana (AstHMGR+QsbAS+QsCYP716-C-28+QsCYP716-C-16α+QsCYP714-C-23+QsCsIG2+Qs-3-O-GalT+Qs_0283850+Qs-28-O-FucT). In addition, co-expression of green fluorescent protein (GFP, negative control), QsFucSyn, ATCV-1 or both QsFucSyn and ATCV-1 together was performed. Following the transient expression, the relative levels of QA-TriR and QA-TriR-F were measured in N. benthamiana leaf extracts by LC-CAD relative to the internal standard (digitoxin, 1.1 μg/mg dry leaf). All samples were measured in triplicate (n=3). Error bars denote standard deviation.

FIG. 22 shows a comparison of the impact of co-expression of the QsFucSyn-Like enzymes on QA-TriR-F yields. The gene set necessary for production of QA-TriR-F was transiently co-expressed in N. benthamiana (AstHMGR+QsbAS+QsCYP716-C-28+QsCYP716-C-16α+QsCYP714-C-23+QsCsIG2+Qs-3-O-GalT+Qs_0283850+Qs-28-O-FucT). In addition, either green fluorescent protein (GFP, negative control), QsFucSyn (positive control), or one of three FucSyn-Like proteins from Q. saponaria (QsFSL-1 and QsFSL-2) or Saponaria officinalis (SoFSL-1) were co-expressed. Following the transient expression, the relative level of QA-TriR-F was measured in N. benthamiana leaf extracts by LC-CAD relative to the internal standard (digitoxin, 1.1 μg/mg dry leaf).

FIG. 23 shows that QsFucSyn and homologues (i.e. FucSyn-Like proteins) are likely to be SDR114C family members. Phylogenetic analysis was conducted using the Neighbour Joining method (Saitou & Nei, 1987 in MegaX (Kumar et al., 2016). Node labels show bootstrap value percentages (5000 replicates). Accession numbers for genes used in the tree are: M. piperita Menthol dehydrogenase (AAQ55960), M. pipertia Neomenthol dehydrogenase (AAQ55959), M. piperita Isopiperitenone reductase (AAQ75422), C. annuum Menthone reductase (ABU54321), A. thaliana CytADR1 (NP_001190151), A. thaliana CytADR2 (NP_179996), P. bracteatum Salutaridine reductase (A4UHT7), A. thaliana Hydroxysteroid dehydrogenase (NP_568742), A. thaliana Tropinone reductase-like (NP_196225) O. sativa MAS (XP_015634207), M. piperita Isopiperitenol dehydrogenase (AAU20370), A. thaliana ADH (NP_566097), S. lycopersicum GAME25 (NP_001233856), D. lanata 3Hydroxysteroid reductase (AAW31720), A. thaliana Pinoresinol reductase1 (Q9FVQ6), O. basilicum Eugenol synthase1 (Q15G14), M. sativa Isoflavone reductase (P52575), Z. mays Leucoanthocyanin reductase (ACG33275), A. thaliana Anthocyanidin reductase (NP_176365), M. sativa Vestitone reductase (Q40316), P. somniferum Noscapine synthase (I3PLR3), A. thaliana Dihydroflavanol-4-reductase (XP_020884177), M. truncatula 6-deoxychalcone synthase (XP_003618003), P. somniferum Codeinone reductase (Q9SQ70), A. thaliana Aldo-Keto Reductase (NP_176203).

FIG. 24 shows the spinach Yossoside I pathway and boosting effects by SpoIFSL A) The spinach Yossoside I biosynthetic pathway. The SOAP6 gene catalyses D-fucosylation of medicagenic acid 3-O-glucuronoside to form Yossoside I. B) Transient expression of the spinach FucSyn-like (SpoIFSL) enzyme with the Yossoside I gene set results in enhanced Yossoside I accumulation in N. benthamiana. Data are shown as LC-MS extract ion chromatograms (EIC) for m/z 823 (Yossoside I) and m/z 809 (Internal standard digitoxin). The top panel represents the Yossoside gene set without the SOAP6 D-fucosyltransferase (AstHMGR/QsbAS/QsCYP716-C-28/SOAP3/SOAP4/SOAP5). The middle panel shows the small accumulation of Yossoside I (m/z 823, 12.3 min) when SOAP6 is included. The bottom panel shows the boost in Yossoside I when including the spinach Fucsyn-like enzyme SpoIFSL. C) Quantification of Yossoside I content (based on LC-CAD peak area) when the full Yossoside I gene set is transiently expressed in N. benthamiana either alone (left) or with either the spinach SpoIFSL (middle) or the QsFucSyn enzyme (right).

FIG. 25 shows the impact of SpoIFSL and other FucSyn-like proteins on boosting QA-TriR-F content. A) The gene set for production of QA-TriR-F (AstHMGR/QsbAS/QsCYP716-C-28/QsCYP716-C-16α/QsCYP714-C-23/QsCSL2/Qs-3-O-GalT/Qs-3-O-RhaT/Qs-28-O-FucT) was transiently expressed in N. benthamiana. In addition, the various FucSyn proteins from Quillaja saponaria (FucSyn (QsFucSyn), FucSyn-like 1 (QsFSL-1) and FucSyn-like 2 (QsFSL-2)), Spinacia oleracea (SpoIFSL) and Saponaria officinalis (SoFSL) were co-expressed and the impact of these genes on QA-TriR-F content was measured by LC-CAD. B) Protein pairwise percentage sequence identities between the various FucSyn-like proteins.

FIG. 26 shows 1H and 13C-NMR spectroscopic data for Quillaic acid 3-O-{α-L-rhamnopyranosyl-(1→3)-[β-D-galactopyranosyl-(1→2)]-β-D-glucopyranosiduronic acid}-28-O-[β-D-fucopyranosyl] (QA-TriR-F) in MeOH-d₄, (600, 150 MHz).

FIG. 27 shows ¹H and ¹³C-NMR spectroscopic data for Quillaic acid 3-O-{α-L-rhamnopyranosyl-(1→3)-[β-D-galactopyranosyl-(1→2)]-β-D-glucopyranosiduronic acid}-28-O-{[α-L-rhamnopyranosyl-(1→2)-[β-D-fucopyranosyl]} (QA-TriR-FR) in MeOH-d₄, (600, 150 MHz).

FIG. 28 shows ¹H and ¹³C-NMR spectroscopic data for Quillaic acid 3-O-{α-L-rhamnopyranosyl-(1→3)-[β-D-galactopyranosyl-(1→2)]-β-D-glucopyranosiduronic acid}-28-O-{[β-D-xylopyranosyl-(1→4)-α-L-rhamnopyranosyl-(1→2)-[β-D-fucopyranosyl]} (QA-TriR-FRX) in MeOH-d₄/D₂O, 10:1 (600, 150 MHz).

FIG. 29 shows ¹H and ¹³C-NMR spectroscopic data for Quillaic acid 3-O-{α-L-rhamnopyranosyl-(1→3)-[β-D-galactopyranosyl-(1→2)]-β-D-glucopyranosiduronic acid}-28-O-{[β-D-xylopyranosyl-(1→3)-[β-D-xylopyranosyl-(1→4)-α-L-rhamnopyranosyl-(1→2)-[β-D-fucopyranosyl]} (QA-TriR-FRXX) in MeOH-d₄/D₂O, 10:1 (600, 150 MHz).

FIG. 30 shows ¹H and ¹³C-NMR spectroscopic data for Quillaic acid 3-O-{α-L-rhamnopyranosyl-(1→3)-1-[β-D-galactopyranosyl-(1→2)]-β-D-glucopyranosiduronic acid}-28-O-{[β-D-apiofuranosyl-(1→3)-[β-D-xylopyranosyl-(1-4)-α-L-rhamnopyranosyl-(1→2)-[β-D-fucopyranosyl]} (QA-TriR-FRXA) in MeOH-d₄/D₂O, 10:1 (600, 150 MHz).

### DETAILED DESCRIPTION OF THE INVENTION

A first aspect of the invention is a method of making QA-FRX(X/A), wherein the FRX(X/A) chain is added to the C-28 position of QA, the method comprising:
(i) (a) combining QA with UDP-α-D-fucose and the enzyme Qs-28-O-FucT (SEQ ID NO 2) or an enzyme with a sequence with at least 70% sequence identity and/or
   (b) combining QA with UDP-4-keto, 6-deoxy-D-glucose, the enzyme Qs-28-O-FucT (SEQ ID NO 2) or an enzyme with a sequence with at least 70% sequence identity, and the enzyme QsFucSyn (SEQ ID NO 12) or an enzyme with a sequence with at least 45% sequence identity to form QA-F; then
(ii) combining QA-F with UDP-β-L-rhamnose and the enzyme Qs-28-O-RhaT (SEQ ID NO 4) or an enzyme with a sequence with at least 70% sequence identity to form QA-FR; (iii) combining QA-FR with UDP-α-D-xylose and the enzyme Qs-28-O-XylT3 (SEQ ID NO 6) or an enzyme with a sequence with at least 70% sequence identity to form QA-FRX; and
(iv) combining QA-FRX either with UDP-α-D-xylose and the enzyme Qs-28-O-XylT4 (SEQ ID NO 8) or an enzyme with a sequence with at least 70% sequence identity to form QA-FRXX, and/or combining QA-FRX with UDP-α-D-apiose and the enzyme Qs-28-O-ApiT4 (SEQ ID NO 10) or an enzyme with a sequence with at least 70% sequence identity to form QA-FRXA.

The percentage sequence identities discussed in this application are the percentage sequence identities across the full length of the sequences identified by the SEQ. ID NOs. This may include shortened sequences which have the same sequence identity measured across the length of the shortened sequence. The shortened sequences may have the same homology of the percentage sequence identity of the SEQ. ID. NO. regardless of the length of the shortened sequence. The shortened sequence may be at least half the length of the full-length sequence, preferably at least three quarters of the length of the full sequence.

In this aspect of the invention, the sugar donors are UDP-sugars. If the sugar donors are free sugars they are converted to UDP-sugars, before being used in the method of the first aspect of the invention.

Preferably, the method of the first aspect of the invention is carried out in a biological system. The biological system is a plant or a microorganism wherein nucleic acids encoding one or more of the enzymes of the first aspect of the invention are introduced. In most cases, the biological system will not naturally express any of the enzymes of the first aspect of the invention and thus the biological system will be engineered to express all five enzymes. If the host does not naturally produce the required UDP-sugars as required for the first aspect of the invention, the system will also be engineered to produce such sugars. Preferably, the biological system either naturally produces such sugars (e.g. N. benthamiana), or can be engineered to produce such sugars, e.g. yeast.

In N. benthamiana, many UDP-sugars (e.g. UDP-rhamnose) are naturally present in the plants. The UGT (UDP-dependent glycosyltransferases) enzymes of the first aspect of the invention are engineered to be expressed by the plant and the pathway to biosynthetically produce a QA derivative is obtained. A UDP-sugar may be present, but not in high amounts, therefore limiting the amount of product produced. For example, UDP-α-D-apiose and UDP-α-D-fucose may not be present in high amount in N. benthamiana. One way to address this and increase the levels of these sugars is to also engineer the host plant to produce more of the sugar and/or by engineering it to express one or more boosting enzymes. The boosting enzyme for UDP-α-D-apiose may be QsAXS1 (SEQ ID No. 14). The boosting enzymes for UDP-α-D-fucose may be QsFucSyn (SEQ ID No. 12), ATCV-1 (SEQ. ID No 40) or QsFucSyn-Like enzymes, such as QsFSL-1 (SEQ ID No. 48), QsFSL-2 (SEQ ID No 50), SoFSL-1 (SEQ ID No 52) or SpolFSL (SEQ ID NO 54), discussed below. If UDP-α-D-fucose is not present in high amounts, another way to address this is to combine QA with UDP-4-keto, 6-deoxy-D-glucose, Qs-28-O-FucT (SEQ ID NO 2) or an enzyme with a sequence with at least 70% sequence identity, and QsFucSyn (SEQ ID NO 12) or an enzyme with a sequence with at least 45% sequence identity to form QA-F.

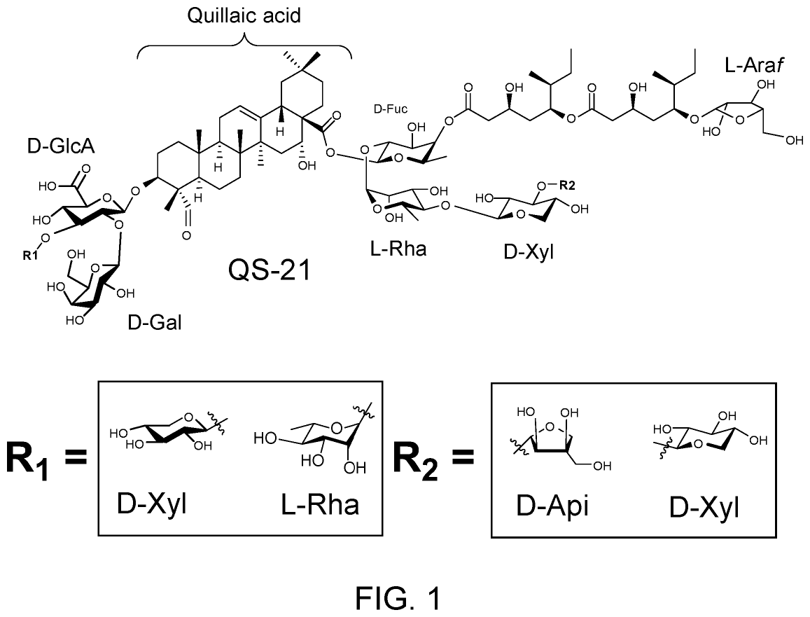

QA-Tri(X/R)-FRX(X/A) or QA-FRX(X/A) is formed by the sequential addition, to the QA backbone, of the sugar units forming the C-28 tetrasaccharide chain as described in FIG. 1. The linear tetrasaccharide at the C-28 position of the QA core is initiated by attaching D-fucose with a β-linkage to a molecule comprising QA to form a molecule comprising QA-F. This step is followed by attaching L-rhamnose with an α-linkage to the molecule comprising QA-F, to produce a molecule comprising QA-FR. Next, D-xylose is attached with a β-linkage to a molecule comprising QA-FR to produce a molecule comprising QA-FRX. Finally, D-xylose is attached with a β-linkage to a molecule comprising QA-FRX to produce a molecule comprising QA-FRXX or D-apiose is attached with a β-linkage to a molecule comprising QA-FRX to produce a molecule comprising QA-FRXA.

In the following description, the method of the invention is described for the situation when the linear tetrasaccharide at the C-28 position of the molecule comprising the QA core is initiated by attaching D-fucose with a β-linkage to a molecule comprising QA to form a molecule comprising QA-F.

The method is preferably performed such that the molecule comprising QA-FRX(X/R), can be isolated or further derivatized to chemically synthesise downstream products, such as QS-21.

In this aspect of the invention, the QA derivative is QA-FRXX (or QA-Tri(X/R)-FRXX) or QA-FRXA (or QA-Tri(X/R)-FRXA) or a mixture comprising QA-FRXX and QA-FRXA (or QA-Tri(X/R)-FRXX and QA-Tri(X/R)-FRXA). When the QA derivative is a mixture comprising QA-FRXX and QA-FRXA (or QA-Tri(X/R)-FRXX and QA-Tri(X/R)-FRXA), the ratio of QA-FRXX to QA-FRXA (or QA-Tri(X/R)-FRXX to QA-Tri(X/R)-FRXA) may vary. The ratio of QA-FRXX to QA-FRXA (or QA-Tri(X/R)-FRXX to QA-Tri(X/R)-FRXA) within the mixture may vary in percentage. Suitably, the mixture comprises from 10% to 90% of QA-FRXX (or QA-Tri(X/R)-FRXX), such as 10%, 20%, 30%, 40%, 50%, 60%, 70%, 80%, or 90% and from 90 to 10% of QA-FRXA (or QA-Tri(X/R)-FRXA), such as 90%, 80%, 70%, 60%, 50%, 40%, 30%, 20%, or 10%. Preferably, the mixture comprises 60% of QA-FRXX (or QA-Tri(X/R)-FRXX) and 40% of QA-FRXA (or QA-Tri(X/R)-FRXA), or 50% of each.

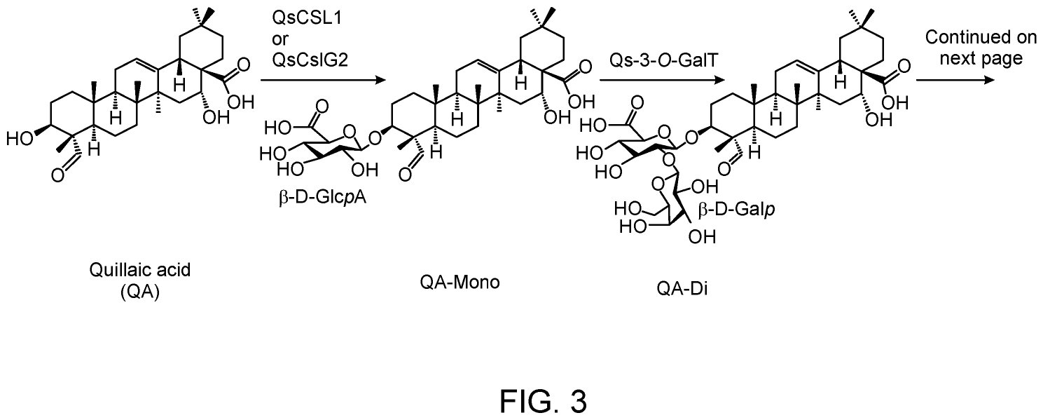

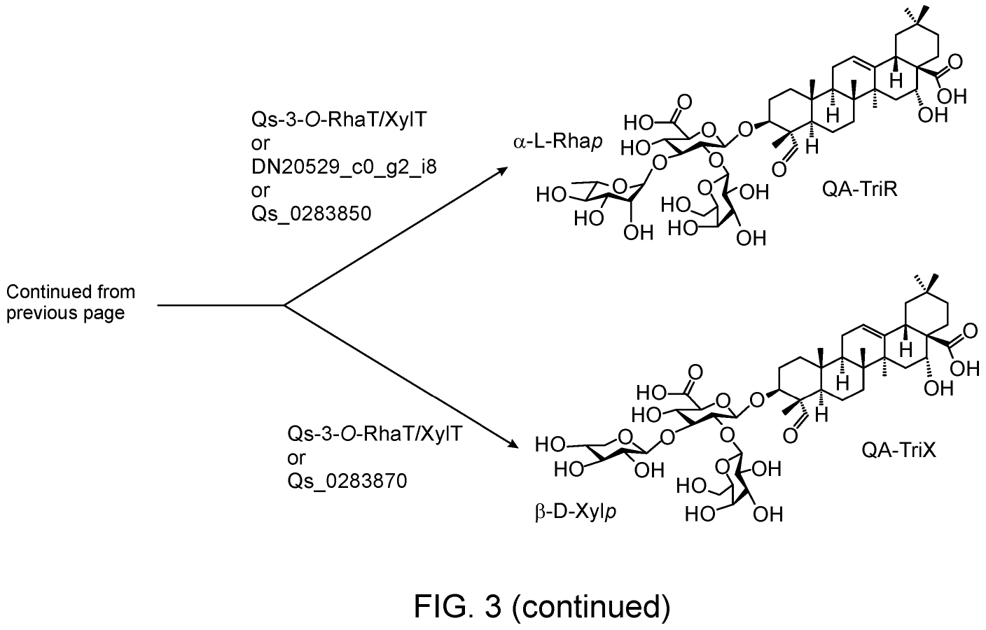

In QA-TriR or QA-TriX, the sugar attached to the C-3 position is β-D-glucuronic acid (GlcpA) as shown in FIG. 3. The GlcpA residue may have two sugars linked to it. One sugar linked to the GlcpA residue is a D-galactopyranose (Galp). The D-galactopyranose may be attached with a β-1,2-linkage. One sugar linked to the GlcpA residue may be either a D-xylopyranose (Xylp) or an L-rhamnopyranose (Rhap). The D-xylopyranose or L-rhamnopyranose may be attached with a β-1,3-linkage or an α-1,3-linkage, respectively.

The first step of the method of the first aspect of the invention is attaching D-fucose with a β-linkage to a molecule comprising QA, which molecule may be QA-TriR and/or QA-TriX. This step is carried out by the enzyme Qs-28-O-FucT (SEQ ID NO 2) or by an enzyme with a sequence with at least 70% sequence identity to Qs-28-O-FucT. The enzyme is capable of transferring D-fucose with a β-linkage to the C-28 position of a molecule comprising QA. The function of the enzyme can be determined for example by transient expression in N. benthamiana as described in Materials and Methods and Example 2.

Briefly, co-expression of the gene encoding the enzyme to be tested along with the genes required to produce a molecule such as QA-TriX (see PCT/EP2020/067866 published as WO 2020/260475) (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283870 (SEQ ID NO 37)) or QA-TriR (see PCT/EP2020/067866)⁴ (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283850 (SEQ ID NO 35)) should result in the production of the fucosylated products, QA-TriX-F (monoisotopic mass=1102.52, [M-H]⁻=1101) or QA-TriR-F (monoisotopic mass=1116.54, [M-H]⁻=1115), respectively. The identity of the product can be confirmed by a large-scale infiltration, purification of the product and confirmation of the structure by NMR as described in Materials and Methods, alternatively, the identity of the product could be confirmed by LC-MS as described in Materials and Methods, and comparison of the retention time and mass of the peak obtained with a standard of QA-TriX-F or QA-TriR-F, or by comparison with the product obtained by the co-expression of the above genes required to produce QA-TriX or QA-TriR with the gene for the fucosyltransferase Qs-28-O-FucT (SEQ ID NO 1).

The percentage sequence identity of the sequence for the enzyme Qs-28-O-FucT may vary. The sequence identity may be at least 70%, 75%, 80%, 85%, 90%, or 95% identity to SEQ ID NO 2. Accordingly, in some embodiments, the enzyme Qs-28-O-FucT used in the methods of the invention has at least 70%, 75%, 80%, 85%, 90%, or 95% sequence identity to SEQ ID NO 2, suitably at least 90%, more suitably at least 95%.

An alternative first step of the method of the first aspect of the invention is attaching UDP-4-keto, 6-deoxy-D-glucose to a molecule comprising QA, which molecule may be QA-TriR and/or QA-TriX, then carrying out a keto-reduction at the C-4 position. This step is carried out by the enzyme Qs-28-O-FucT (SEQ ID NO 2), or by an enzyme with a sequence with at least 70% sequence identity to Qs-28-O-FucT, and the enzyme QsFucSyn (SEQ ID NO 12), or an enzyme with a sequence with at least 45% sequence identity to QsFucSyn. This step is discussed in more detail in relation to the second aspect of the invention.

The second step of the method of the first aspect of the invention is attaching α-L-rhamnose to a β-D-fucose residue. This step is carried out by the enzyme Qs-28-O-RhaT (SEQ ID NO 4) or an enzyme having a sequence with at least 70% sequence identity to Qs-28-O-RhaT. The enzyme is capable of transferring L-rhamnose to a D-fucose residue. The function of the enzyme can be determined for example by transient expression in N. benthamiana as described in Materials and Methods and Example 3. Briefly, co-expression of the gene encoding the enzyme to be tested along with the genes required to produce a molecule such as QA-TriX-F (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283870 (SEQ ID NO 37), Qs-28-O-FucT (SEQ ID NO 1)) or QA-TriR-F (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283850 (SEQ ID NO 35), Qs-28-O-FucT (SEQ ID NO 1)) should result in the production of the rhamnosylated products, QA-TriX-FR (monoisotopic mass=1248.58, [M-H]⁻=1247) or QA-TriR-FR (monoisotopic mass=1262.59, [M-H]⁻=1261), respectively. The identity of the product can be confirmed by a large-scale infiltration, purification of the product and confirmation of the structure by NMR as described in Materials and Methods, alternatively, the identity of the product could be confirmed by LC-MS as described in Materials and Methods and comparison of the retention time and mass of the peak obtained with a standard of QA-TriX-FR or QA-TriR-FR, or by comparison with the product obtained by the co-expression of the above genes required to produce QA-TriX-F or QA-TriR-F with the gene for the rhamnosyltransferase Qs-28-O-RhaT (SEQ ID NO 3).

The percentage sequence identity of the sequence for the enzyme Qs-28-O-RhaT may vary. The sequence identity may be at least 70%, 75%, 80%, 85%, 90%, or 95% identity to SEQ ID NO 4. Accordingly, in some embodiments, the enzyme Qs-28-O-RhaT used in the methods of the invention has at least 70%, 75%, 80%, 85%, 90%, or 95% sequence identity to SEQ ID NO 4, suitably at least 90%, more suitably at least 95%.

The third step of the method of the first aspect of the invention is attaching β-D-xylose to a α-L-rhamnose residue. This step is carried out by the enzyme Qs-28-O-XylT3 (SEQ ID NO 6) or by an enzyme with a sequence with at least 70% sequence identity to Qs-28-O-XylT3. The enzyme is capable of transferring D-xylose. The function of the enzyme can be determined for example by transient expression in N. benthamiana as described in Materials and Methods and Example 4. Briefly, co-expression of the gene encoding the enzyme to be tested along with the genes required to produce a molecule such as QA-TriX-FR (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283870 (SEQ ID NO 37), Qs-28-O-FucT (SEQ ID NO 1), Qs-28-O-RhaT (SEQ ID NO 3)) or QA-TriR-FR (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283850 (SEQ ID NO 35), Qs-28-O-FucT (SEQ ID NO 1), Qs-28-O-RhaT (SEQ ID NO 3)) should result in the production of the xylosylated products, QA-TriX-FRX (monoisotopic mass=1380.62, [M-H]⁻=1379) or QA-TriR-FRX (monoisotopic mass=1394.64, [M-H]⁻=1393), respectively. The identity of the product can be confirmed by a large-scale infiltration, purification of the product and confirmation of the structure by NMR as described in Materials and Methods, alternatively, the identity of the product could be confirmed by LC-MS as described in Materials and Methods and comparison of the retention time and mass of the peak obtained with a standard of QA-TriX-FRX or QA-TriR-FRX, or by comparison with the product obtained by the co-expression of the above genes required to produce QA-TriX-FR or QA-TriR-FR with the gene for the xylosyltransferase Qs-28-O-XylT3 (SEQ ID NO 5).

The percentage sequence identity of the sequence for the enzyme Qs-28-O-XylT3 may vary. The sequence identity may be at least 70%, 75%, 80%, 85%, 90%, or 95% identity to SEQ ID NO 6. Accordingly, in some embodiments, the enzyme Qs-28-O-XylT3 used in the methods of the invention has at least 70%, 75%, 80%, 85%, 90%, or 95% sequence identity to SEQ ID NO 6, suitably at least 90%, more suitably at least 95%.

A fourth step of the method of the first aspect of the invention is attaching β-D-xylose to a β-D-xylose residue. This step is carried out by the enzyme Qs-28-O-XylT4 (SEQ ID NO 8) or by an enzyme having a sequence with at least 70% sequence identity to Qs-28-O-XylT4. The enzyme is capable of transferring D-xylose. The function of the enzyme can be determined for example by transient expression in N. benthamiana as described in Materials and Methods and Example 5. Briefly, co-expression of the gene encoding the enzyme to be tested along with the genes required to produce a molecule such as QA-TriX-FRX (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283870 (SEQ ID NO 37), Qs-28-O-FucT (SEQ ID NO 1), Qs-28-O-RhaT (SEQ ID NO 3), Qs-28-O-XylT3 (SEQ ID NO 5)) or QA-TriR-FRX (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283850 (SEQ ID NO 35), Qs-28-O-FucT (SEQ ID NO 1), Qs-28-O-RhaT (SEQ ID NO 3), Qs-28-O-XylT3 (SEQ ID NO 5)) should result in the production of the xylosylated products, QA-TriX-FRXX (monoisotopic mass=1512.66, [M-H]⁻=1511) or QA-TriR-FRXX (monoisotopic mass=1526.68, [M-H]⁻=1525), respectively. The identity of the product can be confirmed by a large-scale infiltration, purification of the product and confirmation of the structure by NMR as described in Materials and Methods, alternatively, the identity of the product could be confirmed by LC-MS as described in Materials and Methods and comparison of the retention time and mass of the peak obtained with a standard of QA-TriX-FRXX or QA-TriR-FRXX, or by comparison with the product obtained by the co-expression of the above genes required to produce QA-TriX-FRX or QA-TriR-FRX with the gene for the xylosyltransferase Qs-28-O-XylT4 (SEQ ID NO 7).

The percentage sequence identity of the sequence for the enzyme Qs-28-O-XylT4 may vary. The sequence identity may be at least 70%, 75%, 80%, 85%, 90%, or 95% identity to SEQ ID NO 8. Accordingly, in some embodiments, the enzyme Qs-28-O-XylT4 used in the methods of the invention has at least 70%, 75%, 80%, 85%, 90%, or 95% sequence identity to SEQ ID NO 8, suitably at least 90%, more suitably at least 95%.

An alternative fourth step of the method of the first aspect of the invention is attaching β-D-apiose to a β-D-xylose residue. This step is carried out by the enzyme Qs-28-O-ApiT4 (SEQ ID NO 10) or an enzyme having a sequence with at least 70% sequence identity to Qs-28-O-ApiT4. The enzyme is preferably capable of transferring D-apiose. The function of the enzyme can be determined for example by transient expression in N. benthamiana as described in Materials and Methods and Example 5. Briefly, co-expression of the gene encoding the enzyme to be tested along with the gene to encode QsAXS1 (SEQ ID NO 13) and the genes required to produce a molecule such as QA-TriX-FRX (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283870 (SEQ ID NO 37), Qs-28-O-FucT (SEQ ID NO 1), Qs-28-O-RhaT (SEQ ID NO 3), Qs-28-O-XylT3 (SEQ ID NO 5)) or QA-TriR-FRX (such as AstHMGR (SEQ ID No 15), QsbAS (SEQ ID NO 17), QsCYP716-C-28 (SEQ ID NO 19), QsCYP716-C-16a (SEQ ID NO 21), QsCYP714-C-23 (SEQ ID NO 23), CsIG2 (SEQ ID NO 27), Qs-3-O-GalT (SEQ ID NO 29), Qs_0283850 (SEQ ID NO 35), Qs-28-O-FucT (SEQ ID NO 1), Qs-28-O-RhaT (SEQ ID NO 3), Qs-28-O-XylT3 (SEQ ID NO 5)) should result in the production of the apiosylated products, QA-TriX-FRXA (monoisotopic mass=1512.66, [M-H]⁻=1511) or QA-TriR-FRXA (monoisotopic mass=1526.68, [M-H]⁻=1525), respectively. The identity of the product can be confirmed by a large-scale infiltration, purification of the product and confirmation of the structure by NMR as described in Materials and Methods, alternatively, the identity of the product could be confirmed by LC-MS as described in Materials and Methods and comparison of the retention time and mass of the peak obtained with a standard of QA-TriX-FRXA or QA-TriR-FRXA, or by comparison with the product obtained by the co-expression of the above genes required to produce QA-TriX-FRX or QA-TriR-FRX with the gene for QsAXS1 (SEQ ID NO 13) and the apiosyltransferase Qs-28-O-ApiT4 (SEQ ID NO 9).

The percentage sequence identity of the sequence for the enzyme Qs-28-O-ApiT4 may vary. The sequence identity may be at least 70%, 75%, 80%, 85%, 90%, or 95% identity to SEQ ID NO 10. Accordingly, in some embodiments, the enzyme Qs-28-O-ApiT4 used in the methods of the invention has at least 70%, 75%, 80%, 85%, 90%, or 95% sequence identity to SEQ ID NO 10, suitably at least 90%, more suitably at least 95%.

The percentage sequence identity of the sequences to Qs-28-O-FucT, Qs-28-O-RhaT, Qs-28-O-XylT3, Qs-28-O-ApiT4 and Qs-28-O-ApiT4 may all be the same or different.

The method of the first aspect of the invention may be performed in vitro. By “in vitro”, it is meant in the sense of the present invention to have appropriate QA derivatives enzymatically treated with appropriate enzymes of the invention. QA derivatives may be either biosynthetically produced or chemically synthesized. Enzymes may be either chemically synthesized or purified from their native environment. It is within the skilled person's ambit to determine the optimal conditions (e.g. duration, temperature, buffer etc.) of the enzymatic treatment. The identity of the QA derivative can be confirmed, for example, by elucidating its structure by NMR as described in Materials and Methods. In one embodiment, the in vitro method of the first aspect of the invention to make QA-FRX(X/A) comprises to have a molecule comprising QA (e.g. QA or QA-Tri(X/R)) enzymatically treated with a mixture of enzymes comprising Qs-28-O-FucT (SEQ ID NO 2), Qs-28-O-RhaT (SEQ ID NO 4), Qs-28-O-XylT3 (SEQ ID NO 6), Qs-28-O-XylT4 (SEQ ID NO 8) and Qs-28-O-ApiT4 (SEQ ID NO 10), in the presence of UDP-α-D-fucose, UDP-β-L-rhamnose, UDP-α-D-xylose and UDP-α-D-apiose.

Preferably, the method of the first aspect of the invention is carried out in a biological system. The nucleic acids encoding for one or more of the above enzymes are introduced and expressed in the biological system.

The biological system may be a plant or a microorganism. When the biological system is a plant, the plant may be row crops for example sunflower, potato, canola, dry bean, field pea, flax, safflower, buckwheat, cotton, maize, soybeans and sugar beets. The plant may also be corn, wheat, oilseed rape and rice. Preferably the plant may be Nicotiana benthamiana.

In certain aspects of the invention, the biological system is not Quillaja saponaria.

When the biological system is a microorganism, the microorganism may be bacteria or yeast.

Yeast (Saccharomyces cerevisiae) is a heterologous host used for the production of high value small molecules, including terpenes. Like plants, yeast endogenously produces the triterpenoid precursor 2,3-oxidosqualene, and so is a promising host for industrial-scale production of triterpenoids. It is also a highly effective host for the functional expression of plant CYPs at endoplasmic reticulum membranes. There is minimal modification of triterpenoid scaffolds by endogenous yeast enzymes, facilitating product purification. Yeast can be a production host producing triterpenes with diverse glycoside conjugates comprising multiple types of sugars in linear and branched configuration. Glycosylation reactions in yeast are restricted by the limited palette of endogenous sugar donors. By expressing genes from higher plants, however, the nucleotide sugar metabolism of yeast can be expanded beyond UDP-glucose and UDP-galactose, to include UDP-rhamnose, -glucuronic acid, -xylose, -arabinose and others.

The method of the first aspect of the invention includes transforming the host with nucleic acids by introducing the nucleic acids required for the biosynthesis of a molecule comprising QA-FRXX/A into the host cells via a vector. Recombination may occur between the vector and the host cell genome to introduce the nucleic acids into the host cell genome.

In one embodiment, there is provided a method of making QA-Mono-FRX(X/A), QA-Di-FRX(X/A) and/or QA-Tri(X/R)-FRX(X/A), wherein the Mono, Di or Tri(X/R) chain is added at the C-3 position and the FRX(X/A) chain is added at the C-28 position of QA, the method comprising:
(i) combining QA with UDP-α-D-glucopyranuronic acid and the enzyme QsCSL1 (SEQ ID NO 26) or QsCsIG2 (SEQ ID NO 28) or an enzyme with a sequence with at least 70% sequence identity to form QA-Mono; optionally
(ii) combining QA-Mono with UDP-α-D-galactopyranose and the enzyme Qs-3-O-GalT (SEQ ID NO 30) or an enzyme with a sequence with at least 70% sequence identity to form QA-Di; optionally
(iii) combining QA-Di with UDP-β-L-rhamnopyranose and the enzyme DN20529_c0_g2_i8 (SEQ ID NO 36) or Qs_0283850 (SEQ ID NO 34), or Qs-3-O-RhaT/XylT (SEQ ID NO 32) or an enzyme with a sequence with at least 70% sequence identity to form QA-TriR, and/or combining QA-Di with UDP-α-D-xylopyranose and the enzyme Qs_0283870 (SEQ ID NO 38) or Qs-3-O-RhaT/XylT (SEQ ID NO 32) or an enzyme with a sequence with at least 70% sequence identity to form QA-TriX;
(iv) (a) combining QA-Mono, QA-Di and/or QA-Tri(R/X) with UDP-α-D-fucose and the enzyme Qs-28-O-FucT (SEQ ID NO 2) or an enzyme with a sequence with at least 70% sequence identity to form QA-Mono-F, QA-Di-F and/or QA-Tri(R/X)-F, and/or
   (b) combining QA-Mono, QA-Di and/or QA-Tri(R/X) with UDP-4-keto, 6-deoxy-D-glucose, the enzyme Qs-28-O-FucT (SEQ ID NO 2) or an enzyme with a sequence with at least 70% sequence identity, and of the enzyme QsFucSyn (SEQ ID NO 12) or an enzyme with a sequence with at least 45% sequence identity to form QA-Mono-F, QA-Di-F and/or QA-Tri(R/X)-F;
(v) combining QA-Mono-F, QA-Di-F and/or QA-Tri(R/X)-F with UDP-β-L-rhamnose and the enzyme Qs-28-O-RhaT (SEQ ID NO 4) or an enzyme with a sequence with at least 70% sequence identity to form QA-Mono-FR, QA-Di-FR and/or QA-Tri(R/X)-FR;
(vi) combining QA-Mono-FR, QA-Di-FR and/or QA-Tri(R/X)-FR with UDP-α-D-xylose and the enzyme Qs-28-O-XylT3 (SEQ ID NO 6) or an enzyme with a sequence with at least 70% sequence identity to form QA-Mono-FRX, QA-Di-FRX and/or QA-Tri(R/X)-FRX; and
(vii) combining QA-Mono-FRX, QA-Di-FRX and/or QA-Tri(R/X)-FRX with UDP-α-D-xylose and the enzyme Qs-28-O-XylT4 (SEQ ID NO 8) or an enzyme with a sequence with at least 70% sequence identity to form QA-Mono-FRXX, QA-Di-FRXX and/or QA-Tri(R/X)-FRXX, and/or combining QA-Mono-FRX, QA-Di-FRX and/or QA-Tri(R/X)-FRX with UDP-α-D-apiose and the enzyme Qs-28-O-ApiT4 (SEQ ID NO 10) or an enzyme with a sequence with at least 70% sequence identity to form QA-Mono-FRXA, QA-Di-FRXA and/or QA-Tri(R/X)-FRXA.

In a further embodiment, there is provided a method of making a biosynthetic 3-O-{α-L-rhamnopyranosyl-(1->3)-[β-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid (QA-TriR-FRXA) in a host, which method comprises the steps of: a) expressing genes required for the biosynthesis of QA-TriR and b) introducing a nucleic acid molecule encoding the enzyme Qs-28-O-FucT (SEQ ID NO 2) or an enzyme with a sequence with at least 70% sequence identity to SEQ ID NO 2; the enzyme Qs-28-O-RhaT (SEQ ID NO 4) or an enzyme with a sequence with at least 70% sequence identity to SEQ ID NO 4; the enzyme Qs-28-O-XylT3 (SEQ ID NO 6) or an enzyme with a sequence with at least 70% sequence identity to SEQ ID NO 6; and the enzyme Qs-28-O-ApiT4 (SEQ ID NO 10) or an enzyme with a sequence with at least 70% sequence identity to SEQ ID NO 10, into the host.

In a further embodiment, there is provided a method of making a biosynthetic 3-O-{α-L-rhamnopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriR-FRXX) in a host, which method comprises the steps of: a) expressing genes required for the biosynthesis of QA-TriR, and b) introducing a nucleic acid molecule encoding the enzyme Qs-28-O-FucT (SEQ ID NO 2) or an enzyme with a sequence with at least 70% sequence identity to SEQ ID NO 2; the enzyme Qs-28-O-RhaT (SEQ ID NO 4) or an enzyme with a sequence with at least 70% sequence identity to SEQ ID NO 4; the enzyme Qs-28-O-XylT3 (SEQ ID NO 6) or an enzyme with a sequence with at least 70% sequence identity to SEQ ID NO 6; and the enzyme Qs-28-O-XylT4 (SEQ ID NO 8) or an enzyme with a sequence with at least 70% sequence identity to SEQ ID NO 8, into the host.

In a further embodiment, there is provided a method of making a biosynthetic 3-O-{β-D-xylopyranosyl-(1->3)-[β-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid (QA-TriX-FRXA) in a host, which method comprises the steps of a) expressing genes required for the biosynthesis of QA-TriX and b) introducing a nucleic acid molecule encoding the enzyme Qs-28-O-FucT (SEQ ID NO 2) or an enzyme with a sequence with at least 70% sequence identity to SEQ ID NO 2; the enzyme Qs-28-O-RhaT (SEQ ID NO 4) or an enzyme with a sequence with at least 70% sequence identity to SEQ ID NO 4; the enzyme Qs-28-O-XylT3 (SEQ ID NO 6) or an enzyme with a sequence with at least 70% sequence identity to SEQ ID NO 6; and the enzyme Qs-28-O-ApiT4 (SEQ ID NO 10) or an enzyme with a sequence with at least 70% sequence identity to SEQ ID NO 10, into the host.

In a further embodiment, there is provided a method of making a biosynthetic 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriX-FRXX) in a host, which method comprises the steps of a) expressing genes required for the biosynthesis of QA-TriX, and b) introducing a nucleic acid molecule encoding the enzyme Qs-28-O-FucT (SEQ ID NO 2) or an enzyme with a sequence with at least 70% sequence identity to SEQ ID NO 2; the enzyme Qs-28-O-RhaT (SEQ ID NO 4) or an enzyme with a sequence with at least 70% sequence identity to SEQ ID NO 4; the enzyme Qs-28-O-XylT3 (SEQ ID NO 6) or an enzyme with a sequence with at least 70% sequence identity to SEQ ID NO 6; and the enzyme Qs-28-O-XylT4 (SEQ ID NO 8) or an enzyme with a sequence with at least 70% sequence identity to SEQ ID NO 8, into the host.

In a further embodiment, there is provided a method of making a biosynthetic QA-Tri(X/R)-FRX(X/A)) in a host, which method comprises the steps of a) expressing genes required for the biosynthesis of QA-TriX or QA-TriR, and b) introducing a nucleic acid molecule encoding the enzyme Qs-28-O-FucT (SEQ ID NO 2) or an enzyme with a sequence with at least 70% sequence identity to SEQ ID NO 2; the enzyme Qs-28-O-RhaT (SEQ ID NO 4) or an enzyme with a sequence with at least 70% sequence identity to SEQ ID NO 4; the enzyme Qs-28-O-XylT3 (SEQ ID NO 6) or an enzyme with a sequence with at least 70% sequence identity to SEQ ID NO 6; and, the enzyme Qs-28-O-XylT4 (SEQ ID NO 8 or an enzyme with a sequence with at least 70% sequence identity to SEQ ID NO 8 and/or the enzyme Qs-28-O-ApiT4 (SEQ ID NO 10) or an enzyme with a sequence with at least 70% sequence identity to SEQ ID NO 10, into the host.

The biosynthesis of QA-TriR may be obtained by introducing nucleic acid molecules encoding (i) (a) the enzyme QsCSL1 (SEQ ID NO 26) or an enzyme with a sequence with at least 70% sequence identity to SEQ ID NO 26, or (b) the enzyme QsCsIG2 (SEQ ID NO 28) or an enzyme with a sequence with at least 70% sequence identity to SEQ ID NO 28; (ii) the enzyme Qs-3-O-GalT (SEQ ID NO 30) or an enzyme with a sequence with at least 70% sequence identity to SEQ ID NO 30; and (iii) (a) the enzyme DN20529_c0_g2_i8 (SEQ ID NO 36) or an enzyme with a sequence with at least 70% sequence identity to SEQ ID NO 36, or (b) the enzyme Qs_0283850 (SEQ ID NO 34) or an enzyme with a sequence with at least 70% sequence identity to SEQ ID NO 34, or (c) the enzyme Qs-3-O-RhaT/XylT (SEQ ID NO 32) or an enzyme with a sequence with at least 70% sequence identity to SEQ ID NO 32.

The biosynthesis of QA-TriX may be obtained by introducing nucleic acid molecules encoding (i) (a) the enzyme QsCSL1 (SEQ ID NO 26) or an enzyme with a sequence with at least 70% sequence identity to SEQ ID NO 26, or (b) the enzyme QsCsIG2 (SEQ ID NO 28) or an enzyme with a sequence with at least 70% sequence identity to SEQ ID NO 28; (ii) the enzyme Qs-3-O-GalT (SEQ ID NO 30) or an enzyme with a sequence with at least 70% sequence identity to SEQ ID NO 30; and (iii) (a) the enzyme Qs_0283870 (SEQ ID NO 38) or an enzyme with a sequence with at least 70% sequence identity to SEQ ID NO 38, or (b) the enzyme Qs-3-O-RhaT/XylT (SEQ ID NO 32) or an enzyme with a sequence with at least 70% sequence identity to SEQ ID NO 32.

A second aspect of the invention is an oxidoreductase enzyme according to SEQ ID NO 12 (QsFucSyn) or an enzyme having a sequence with at least 45% sequence identity which is capable of increasing the levels of UDP-α-D-fucose. An enzyme having a sequence with at least 45% sequence identity to SEQ ID NO 12 is not SEQ ID NO 54.

The percentage sequence identity of the sequence for the enzyme QsFucSyn may vary. The sequence identity may be at least 45%, 50%, 55%, 60%, 65%, 70%, 75%, 80%, 85%, 90%, or 95% identity to SEQ ID NO 12.

Alternatively, the oxidoreductase enzyme of the second aspect of the invention with at least 45% sequence identity to SEQ ID NO 12 (QsFucSyn) may be QsFSL-1 (SEQ ID No. 48), QsFSL-2 (SEQ ID No 50) or SoFSL-1 (SEQ ID No 52).

In some hosts, a UDP-sugar may be present, but not in sufficiently high amounts, therefore limiting the amount of product produced. In N. benthamiana UDP-α-D-apiose and UDP-α-D-fucose are not present in high amounts. One way to address this and increase the amount of glycosylated product, for example the apiosylated or fucosylated products, is to increase the levels of the UDP-sugars and/or to use one or more sugar nucleotide biosynthetic enzymes. To increase the amount of apiosylated product, the sugar nucleotide biosynthetic enzyme may be QsAXS1 (SEQ ID No 14). To increase the amount of fucosylated product, the sugar nucleotide biosynthetic enzymes may be QsFucSyn (SEQ ID No 12) or another enzyme possessing UDP-4-keto-6-deoxy-D-glucose 4-keto reductase activity, such as QsFSL-1 (SEQ ID No. 48), QsFSL-2 (SEQ ID No 50), SoFSL-1 (SEQ ID No 52) or SpolFSL (SEQ ID No 54); or ATCV-1 (SEQ. ID No 40).

During work on this invention, it was identified that both co-infiltration of D-fucose or co-expression of QsFucSyn resulted in an improvement to the production of the fucosylated product. The presence of the enzyme was found to increase the production of the fucosylated product.

The QsFucSyn enzyme is an enzyme from Q. saponaria. The QsFucSyn enzyme may be involved in the biosynthesis of UDP-D-fucose. The second step in the proposed biosynthesis of UDP-D-fucose from UDP-D-glucose involves a keto-reduction at the C-4 position. It is expected that the QsFucSyn enzyme is performing this second step, catalysing stereoselective reduction at C-4 of the UDP-4-keto-6-deoxy-D-glucose. Alternatively, the proposed route includes converting UDP-α-D-glucose to a UDP-4-keto-6-deoxy-glucose intermediate. This intermediate is added to the QA backbone then a keto-reduction at the C-4 position occurs to form the fucosylated product. The QsFucSyn enzyme may be reducing the 4-keto group of 4-keto-6-deoxy-glucose after it has been added to the QA backbone.

In a biological system, it may be sufficient to combine a carboxylic acid (for example QA) with UDP-α-D-fucose and a fucosyltransferase enzyme to form the fucosylated product. However, the QsFucSyn enzyme may increase the production of UDP-α-D-fucose, which may lead to a higher yield of the fucosylated product. Indeed, higher abundance of UDP-α-D-fucose allows the fucosyltransferase to operate more efficiently and facilitates more efficient addition of β-D-fucose to a carboxylic acid. Alternatively, UDP-α-D-glucose may be converted to UDP-4-keto-6-deoxy-glucose. The fucosylated product may then be formed by combining a carboxylic acid (for example QA) with UDP-4-keto-6-deoxy-glucose, a fucosyltransferase enzyme and the QsFucSyn enzyme. It is thought that the first step involves adding 4-keto-6-deoxy-glucose (from UDP-4-keto-6-deoxy-glucose) to the QA backbone then reducing the 4-keto group to form the fucosylated product. The QsFucSyn enzyme may reduce the 4-keto group of 4-keto-6-deoxy-glucose after it has been added to the QA backbone. In certain aspects, the QsFucSyn enzyme may also facilitate efficient addition of β-D-fucose to a carboxylic acid at the C-28 position of a molecule comprising QA (for example QA-Tri(X/R)). In certain aspects, the QsFucSyn enzyme may also facilitate efficient reduction of UDP-4-keto-6-deoxy-glucose once it has been added to a carboxylic acid at the C-28 position of a molecule comprising QA (for example QA-Tri(X/R)). Preferably, when a carboxylic acid (such as QA or QA-Tri(X/R)) is combined with UDP-α-D-glucose, a fucosyltransferase enzyme, QsFucSyn and ATCV-1 are combined to form the fucosylated product.

Alternatively, when the reaction takes place in vitro, a carboxylic acid (such as QA or QA-Tri(X/R)) may be treated with a fucosyltransferase enzyme, in the presence of UDP-α-D-fucose, to form the fucosylated product, no QsFucSyn being required. Alternatively, when the reaction takes place in vitro, a carboxylic acid may be treated with a fucosyltransferase enzyme, ATCV-1 and QsFucSyn, in the presence of UDP-α-D-glucose, to form the fucosylated product.

A third aspect of the invention comprises a nucleic acid molecule which encodes the enzyme according to the second aspect of the invention.

The QsFucSyn enzyme may, for example, be encoded by the nucleotide sequence according to SEQ ID NO 11 or by a sequence which, by virtue of the degenerative code, also encodes an enzyme according to the second aspect of the invention.

Each method of the present invention may include combining with the enzyme as set out according to the second aspect of the invention.

Each method of the present invention may include combining with the enzyme as set out according to the second aspect of the invention and the enzyme ATCV-1.

The ATCV-1 enzyme is a UDP-D-glucose 4,6-dehydratase (UGD) and produces UDP-4-keto-6-deoxy-D-glucose from UDP-D-glucose. This represents the first step in UDP-D-fucose biosynthesis (and is also the first step in UDP-L-rhamnose synthesis). As discussed above, the QsFucSyn enzyme may be performing the second step in the proposed biosynthesis of UDP-D-fucose from UDP-D-glucose, catalysing stereoselective reduction at C-4 of the UDP-4-keto-6-deoxy-D-glucose. Alternatively, UDP-4-keto-6-deoxy-glucose is added to the QA backbone then the 4-keto group is reduced to form the fucosylated product. The QsFucSyn enzyme may be performing the 4-keto reduction. Increasing the availability of UDP-4-keto-6-deoxy-D-glucose in N. benthamiana could further enhance the activity of the QsFucSyn enzyme.

Each method of the present invention may include combining with the enzyme as set out according to the second aspect of the invention and combining with one or more enzymes possessing UDP-D-glucose 4,6-dehydratase activity. Such an enzyme could be taken from a UDP-L-rhamnose biosynthetic pathway. The enzyme possessing UDP-D-glucose 4,6-dehydratase activity can be ATCV-1 (SEQ ID No 40) or an enzyme having a sequence with at least 55% sequence identity. The percentage sequence identity of the sequence for ATCV-1 may vary. The sequence identity may be at least 55%, 60%, 65%, 70%, 75%, 80%, 85%, 90%, or 95% identity to SEQ ID NO 40.

When the host contains abundant levels of the required UDP-sugars, the sugar nucleotide biosynthetic enzymes are not required.

Each method of the invention for producing QA-FRX(X/A) (e.g. QA with the C-28 chain) can also include the additional steps of i) including the saccharide units to form the C-3 chain and/or ii) adding the glycosylated C-18 acyl chain, as set out in FIG. 1.

Each method of the invention for producing QA-Tri(R/X)-FRX(X/A) (e.g. QA with the C-3 and C-28 chains) can also include the additional steps of adding the glycosylated C-18 acyl chain, as set out in FIG. 1.

This method involves a number of steps which may be in any order. In summary, the various saccharide chains are attached to a molecule comprising the QA backbone (see FIG. 1) according to the first aspect of the invention. The molecule comprising the QA backbone may be QA-FRXX, QA-FRXA or a mixture of QA-FRXX and QA-FRXA (i.e. QA-FRX(X/A)). Further details of these steps are discussed below.

A fourth aspect of the invention is a fucosyltransferase enzyme according to SEQ ID NO 2 (Qs-28-O-FucT) or an enzyme with a sequence with at least 70% sequence identity. The enzyme is capable of transferring D-fucopyranose with a β-linkage to the C-28 position of a molecule comprising QA. This is an enzyme described in the method of the first aspect of the invention.

The percentage sequence identity of the sequence for Qs-28-O-FucT may vary. The sequence identity may be at least 70%, 75%, 80%, 85%, 90%, or 95% identity to SEQ ID NO 2.

The fucosyltransferase enzyme is encoded by a nucleotide of SEQ ID NO 1 or a nucleic acid molecule which also encodes for the amino acid according to the fourth aspect of the invention.

A fifth aspect of the invention is a rhamnosyltransferase enzyme according to SEQ ID NO 4 (Qs-28-O-RhaT) or an enzyme with a sequence with at least 70% sequence identity.

The enzyme is capable of transferring L-rhamnopyranose with an α-1,2-linkage. This is an enzyme described in the method of the first aspect of the invention.

The percentage sequence identity of the sequence for Qs-28-O-RhaT may vary. The sequence identity may be at least 70%, 75%, 80%, 85%, 90%, or 95% identity to SEQ ID NO 4.

The rhamnosyltransferase enzyme is encoded by a nucleotide of SEQ ID NO 3 or a nucleic acid molecule which also encodes for the amino acid according to the fifth aspect of the invention.

A sixth aspect of the invention is a xylosyltransferase enzyme according to SEQ ID NO 6 (Qs-28-O-XylT3) or an enzyme with a sequence with at least 70% sequence identity. The enzyme is capable of transferring D-xylopyranose with a β-1,4-linkage. This is an enzyme described in the method of the first aspect of the invention.

The percentage sequence identity of the sequence for Qs-28-O-XylT3 may vary. The sequence identity may be at least 70%, 75%, 80%, 85%, 90%, or 95% identity to SEQ ID NO 6.

The xylosyltransferase enzyme of the invention is encoded by a nucleotide of SEQ ID NO 5 or a nucleic acid molecule which also encodes for the amino acid according to the sixth aspect of the invention

A seventh aspect of the invention is a xylosyltransferase enzyme according to SEQ ID NO 8 (Qs-28-O-XylT4) or an enzyme with a sequence with at least 70% sequence identity. This enzyme is capable of transferring D-xylopyranose with a β-1,3-linkage.

The percentage sequence identity of the sequence for Qs-28-O-XylT4 may vary. The sequence identity may be at least 70%, 75%, 80%, 85%, 90%, or 95% identity to SEQ ID NO 8.

The xylosyltransferase enzyme of the invention is encoded by a nucleotide of SEQ ID NO 7 or a nucleic acid molecule which also encode for the amino acid according to the seventh aspect of the invention. This is an enzyme described in the method of the first aspect of the invention.

An eighth aspect of the invention is an apiosyltransferase enzyme according to SEQ ID NO 10 (Qs-28-O-ApiT4) or an enzyme with a sequence with at least 70% sequence identity. This enzyme is capable of transferring D-apiofuranose with a β-1,3-linkage.

The percentage sequence identity of the sequence Qs-28-O-ApiT4 may vary. The sequence identity may be at least 70%, 75%, 80%, 85%, 90%, or 95% identity to SEQ ID NO 10.

The apiosyltransferase enzyme of the invention is encoded by a nucleotide of SEQ ID NO 9 or a nucleic acid molecule which also encodes for the amino acid according to the eighth aspect of the invention. This is an enzyme described in the method of the first aspect of the invention.

Any sequence identity percentage of the fourth, fifth, sixth, seventh and eighth aspects of the invention can be combined with any other sequence identity percentage of the fourth, fifth, sixth, seventh and eighth aspects of the invention.

A ninth aspect of the present invention is a vector comprising one or more of the nucleic acids encoding the enzymes of the fourth to eighth aspects of the invention. The vector may comprise, one, two, three, four or five of the nucleic acids encoding the enzymes of the fourth to eighth aspects of the invention. Preferably, the vector will comprise five of the nucleic acids encoding the enzymes of the fourth to eighth aspects of the invention or a number of vectors which, together, comprise the five nucleic acids. Optionally, the vector may additionally comprise the nucleic acid encoding the enzyme of the second aspect of the invention.

A tenth aspect of the present invention is a host cell comprising the nucleic acids encoding the enzymes of the fourth to eighth aspects of the invention, and optionally, the nucleic acid encoding the enzyme of the second aspect of the invention.

The host cell may be a plant cell or microbial cell. When the host cell is a microbial cell it is preferably a yeast cell. When the host cell is a plant cell, the plant is preferably Nicotiana benthamiana.

An additional feature of the tenth aspect of the invention is the method of introducing the nucleic acids of the fourth to eight aspects of the invention, and optionally the nucleic acid encoding the enzyme of the second aspect of the invention, into the host cell. The nucleic acids may be introduced into the host cells via a vector. Recombination may occur between the vector and host cell genome to introduce the nucleic acids into the host cell genome. Alternatively, the nucleic acids may be introduced into the host cells by co-infiltration with a plurality of recombinant vectors. The recombinant vectors may be Agrobacterium tumefaciens stains, discussed below.

An eleventh aspect of the invention is a biological system comprising host cells as set out according to the tenth aspect of the invention. The biological system may be a plant or a microorganism. When the biological system is a plant, it may be Nicotiana benthamiana or any of the plants described above. The method of producing the plant comprises the steps of introducing the nucleic acids of the invention into the host plant cell and regenerating a plant from the transformed host plant cell. When the biological system is a microorganism, it may be yeast.

The invention also includes the method of making each enzyme and each nucleic acid of the above aspects of the invention, as well as a method of making a vector comprising one or more of the nucleic acids of the invention, as well as the host cells of the tenth aspect of the invention and a method of making the biological system of the eleventh aspect of the invention. These methods use techniques and products well known in the art, such as in WO2019/122259 and PCT/EP2020/067866 (published as WO 2020/260475), and are described in more detail as follows:

The nucleic acids of the invention can be included in a vector, in particular an expression vector, as described in the Example section. The vector may be any plasmid, cosmid, phage or Agrobacterium vector in double or single stranded linear or circular form which can transform a prokaryotic or eukaryotic host either by integration into the cellular genome or other. The vector may be an expression vector, including an inducible promoter, operably linked to the nucleic acid sequence. Typically, the vector may include, between the inducible promoter and the nucleic acid sequence, an enhancer sequence. The vector may also include a terminator sequences and optionally a 3′ UTR located upstream of said terminator sequence. The vector may include one or more nucleic acids encoding enzymes of the first aspect of the invention, preferably all sequences needed to produce one version of the molecule as set out according to the first aspect of the invention. The vector may be a plant vector or a microbial vector.

The nucleic acid in the vector may be under the control of, and operably linked to, an appropriate promoter or other regulatory elements for transcription in a host cell. The host cell may be a yeast cell, bacterial cell or plant cell. The vector may be a bi-functional expression vector which functions in multiple hosts. In the case of genomic DNA, this may contain its own promoter or other regulatory elements. The advantage of using a native promoter is that this may avoid pleiotropic responses. In the case of cDNA this may be under the control of an appropriate promoter or other regulatory elements for expression in the host cell Preferred vectors for use in plants comprise border sequences which permit the transfer and integration of the expression vector into the plant genome. The vector may be a plant binary vector.

The vector may be transfected into a host cell in any biological system. The host may be a microbe, such as E. coli, or yeast. The vector may be part of an Agrobacterium tumefaciens strain and used to infect a biological plant host system. The Agrobacterium tumefaciens may each contain one of the required nucleic acids encoding for the invention and can be combined to co-infect a host cell, such that the host cell contains all the necessary nucleic acids to encode for the enzyme of the first aspect of the invention.

The present invention also includes the steps of culturing the host or growing the host for the production, harvest and isolation of the desired QA derivative.

The QA derivative may require further synthesis, such as addition of the C-18 acyl chain (Wang et al, 2005) To add the C-18 chain via synthetic methods, the QA derivative may be treated with 3-(tert-butyldimethylsilyloxy) propionaldehyde, cis-2-butene, benzyl bromide, tetrabutyl ammonium fluoride, oxalyl chloride, (R)-2-acetoxy-1,1,2-triphenylethanol, sodium methoxide, tert-butyldimethylsilyl chloride (TBSCl), hydrogen, 2,3,5-tri-O-(tert-butyldimethylsilyl)-L-arabinofuranose and barium hydroxide octahydrate.

A method of making the C-18 acyl chain includes the steps of combining 3-(tert-butyldimethylsilyloxy) propionaldehyde with cis-2-butene to make (3S,4S)-6-{[(tert-Butyldimethyl)silyl]oxy}-4-hydroxy-3-methylhex-1-ene. Then combining with benzyl bromide to make (3S,4S)-4-(Benzyl)oxy-6-{[(tert-butyldimethyl)silyl]oxy}-3-methyl-hex-1-ene. The next step includes combining with tetrabutyl ammonium fluoride to make (3S,4S)-4-(Benzyl)oxy 6-hydroxy-3-methylhex-1-ene, then combining with oxalyl chloride to form an aldehyde. The aldehyde is then combined with (R)-2-acetoxy-1,1,2-triphenylethanol then sodium methoxide and TBSCl to form a β-silyloxy methyl ester. The β-Silyloxy methyl ester is then combined with hydrogen to make a methyl ester. The next step includes combining the methyl ester with 2,3,5-tri-O-(tert-butyldimethylsilyl)-L-arabinofuranose to make an arabinoglycoside. The arabinoglycoside is then combined with barium hydroxide octahydrate to make an acid. The next step includes combining the acid with the methyl ester formed previously, to make a diester. The diester is then combined with barium hydroxide octahydrate to make an acid. These steps make the C-18 acyl chain. Once the chain has been made it may be added to the C-28 sugar chain.

A twelfth aspect of the invention is an UDP-apiose/UDP-xylose synthase enzyme according to SEQ ID NO 14 (QsAXS1) or an enzyme with a sequence with at least 70% sequence identity. The enzyme is capable of enhancing the activity of an apiosyltransferase by increasing the availability of the UDP-α-D-apiose when this is limiting.

The percentage sequence identity of the sequence QsAXS1 may vary. The sequence identity may be at least 70%, 75%, 80%, 85%, 90%, or 95% identity to SEQ ID NO 14.

The QsAXS1 enzyme appears to increase the yield of an apiosylated product or a xylosylated product.

For example, the apiosylated product may be a molecule comprising QA-TriX/R-FRXA, or QA-FRXA. β-D-apiose is attached to another sugar residue. The sugar residue may be a β-D-xylose residue. The β-D-xylose residue may be part of a molecule comprising QA-FRX or QA-TriX/R-FRX. This step is carried out by the enzymes Qs-28-O-ApiT4 (SEQ ID NO 10) and QsAXS1 (SEQ ID NO 14) according to the twelfth aspect of the invention.

The xylosylated product may be a molecule comprising QA-TriX/R-FRXX, or QA-FRXX. D-xylose is attached to another sugar residue. The sugar residue may be a β-D-xylose residue. The β-D-xylose residue may be part of a molecule comprising QA-FRX or QA-TriX/R-FRX. This step is carried out by the enzymes Qs-28-O-XylT4 (SEQ ID NO 8) and QsAXS1 (SEQ ID NO 14) according to the twelfth aspect of the invention.

An additional feature of the twelfth aspect of the invention is a nucleic acid molecule which encodes the enzyme of the twelfth aspect of the invention.

The QsAXS1 enzyme may, for example, be encoded by the nucleotide according to SEQ ID NO 13 or by a sequence which, by virtue of the degenerative code, also encodes an enzyme according to the twelfth aspect of the invention.

Each method of the present invention may include combining with the enzyme as set out according to the twelfth aspect of the invention.

An additional feature of the first aspect of the invention is the steps for making the branched trisaccharide at the C-3 position of the molecule comprising the QA core. The method comprises combining a molecule comprising QA with UDP-α-D-glucopyranuronic acid and the enzyme QsCSL1 (SEQ ID NO 26) or the enzyme QsCsIG2 (SEQ ID NO 28); combining with UDP-α-D-galactopyranose and the enzyme Qs-3-O-GalT (SEQ ID NO 30); combining with UDP-β-L-rhamnopyranose and the enzyme DN20529_c0_g2_i8 (SEQ ID NO 36) or the enzyme Qs_0283850 (SEQ ID NO 34), or the enzyme Qs-3-O-RhaT/XylT (SEQ ID NO 32); combining with UDP-α-D-xylopyranose and the enzyme Qs_0283870 (SEQ ID NO 38) or the enzyme Qs-3-O-RhaT/XylT (SEQ ID NO 32).

The sequence identity of each enzyme used in the steps for making the branched trisaccharide at the C-3 position may be at least 50%, 55%, 56%, 57%, 58%, 59%, 60%, 65%, 70% or 80%. Preferably the sequence identity is at least 90%, 95%, 96%, 97%, 98% or 99%.

This feature of the invention relates to a method of making a QA derivative, such as QA-Tri(X/R), involving a number of steps. The steps can be performed in a specific order or in any order or simultaneously. Preferably, this derivative is formed by the sequential addition, to the QA backbone, of the sugar units forming the C-3 chain as discussed below. The sugar units forming the C-28 tetrasaccharide chain are then added according to the first aspect of the invention and as described in FIG. 1.

The steps of this feature of the first aspect of the invention are described for the situation when the branched trisaccharide at the C-3 position of the molecule comprising the QA core is initiated by attaching a β-D-glucopyranuronic acid residue to a molecule comprising QA to form a molecule comprising QA-Mono. However, the steps may occur in any order.

The method is preferably performed such that the molecule comprising QA-TriX/R, can be isolated or further derivatized to chemically synthesise downstream, products, such as QS-21.

One step of the method of the invention is attaching D-glucopyranuronic acid to a molecule comprising QA to form a molecule comprising QA-Mono. The step is carried out by an enzyme QsCSL1 (SEQ ID NO 26) or an enzyme QsCsIG2 (SEQ ID NO 28).

QsCSL1 is encoded by a nucleotide of SEQ ID NO 25. QsCsIG2 is encoded by a nucleotide of SEQ ID NO 27.

Another step of the method of the invention is attaching D-galactopyranose to a β-D-glucopyranuronic acid residue on a molecule comprising QA-Mono to form a molecule comprising QA-Di. The step is carried out by an enzyme Qs-3-O-GalT (SEQ ID NO 30). Qs-3-O-GalT is encoded by a nucleotide of SEQ ID NO 29.

A further step of the method of the invention is attaching L-rhamnopyranose to a β-D-glucopyranuronic acid residue on a molecule comprising QA-Di, to form a molecule comprising QA-TriR. The step is carried out by an enzyme DN20529_c0_g2_i8 (SEQ ID NO 36) or an enzyme Qs_0283850 (SEQ ID NO 34), or an enzyme Qs-3-O-RhaT/XylT (SEQ ID NO 32). DN20529_c0_g2_i8 is encoded by a nucleotide of SEQ ID NO 35. Qs_0283850 is encoded by a nucleotide of SEQ ID NO 33. Qs-3-O-RhaT/XylT, it is encoded by a nucleotide of SEQ ID NO 31.

Yet a further step of the method of the invention involves attaching β-D-xylopyranose to a β-D-glucopyranuronic acid residue on a molecule comprising QA-Di, to form a molecule comprising QA-TriX. The step is carried out by an enzyme Qs_0283870 (SEQ ID NO 38), or an enzyme Qs-3-O-RhaT/XylT (SEQ ID NO 32). Qs_0283870 is encoded by a nucleotide of SEQ ID NO 37. Qs-3-O-RhaT/XylT is encoded by a nucleotide of SEQ ID NO 31.

The steps for adding the sugars of the C-3 trisaccharide and C-28 tetrasaccharide chains to a molecule comprising a QA-core can be performed in a specific order or in any order or simultaneously. Preferably, once the branched trisaccharide at the C-3 position has been attached to a molecule comprising the QA core, the sugar residues of the C-28 tetrasaccharide chain may be added to a molecule comprising QA-TriX, QA-TriR or a mixture of QA-TriX and QA-TriR (i.e. QA-Tri(X/R)), as described in the first aspect of the invention.

An additional feature of the first aspect of the invention is the method steps for making QA. The method comprises combining 2,3 oxidosqualene with QsbAS (SEQ ID NO 18), combining with a C-28 oxidase QsCYP716-C-28 (SEQ ID NO 20), combining with a C-16a oxidase QsCYP716-C-16a (SEQ ID NO 22) and combining with a C-23 oxidase QsCYP714-C-23 (SEQ ID NO 24).

The sequence identity of each enzyme used in the steps for making a molecule comprising the QA core may be at least 50%, 55%, 56%, 57%, 58%, 59%, 60%, 65%, 70% or 80%. Preferably the sequence identity is at least 90%, 95%, 96%, 97%, 98% or 99%.

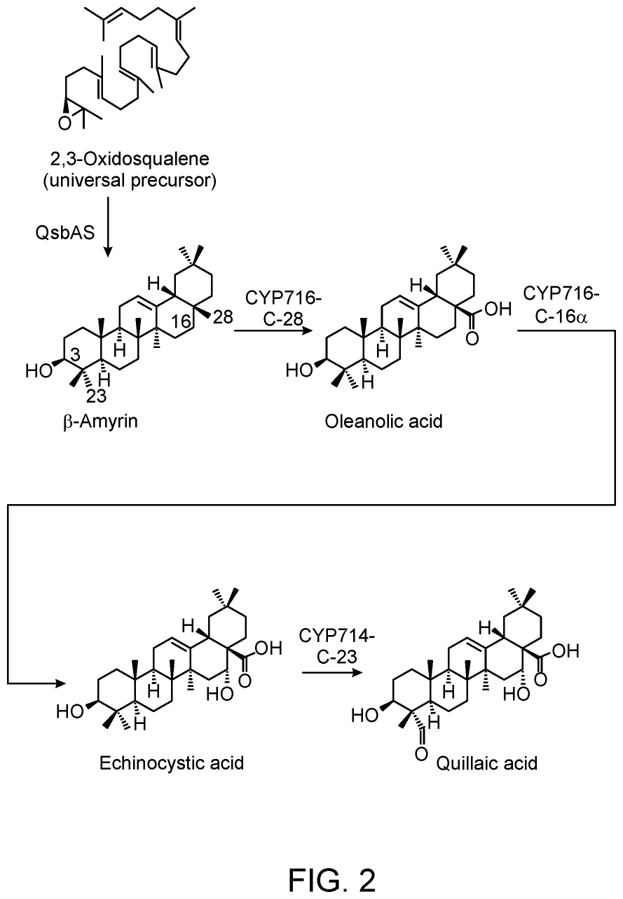

This feature of the invention relates to a method of making a molecule comprising the QA core involving a number of steps. The steps can be performed in a specific order or in any order or simultaneously. Preferably, this molecule is formed by the production of the β-amyrin scaffold followed by the sequential oxidation at the C-28, C-16a and C-23 positions respectively, as described in FIG. 2. The steps of this feature of the first aspect of the invention are described for the preferable situation mentioned above. However, the steps may occur in any order.

The sugar units forming the C-3 trisaccharide and C-28 tetrasaccharide chains are then added according to the first aspect of the invention and as described in FIG. 1. Preferably the molecule comprising the QA core is made then the steps for adding the C-3 chain are carried out, followed by the steps for adding the C-28 tetrasaccharide chain. However, these steps can be performed in a specific order or in any order or simultaneously.

One step of the method of the invention is the cyclisation of 2,3 oxidosqualene to form a molecule comprising triterpene p amyrin. This step is carried out by an oxidosqualene cyclase. In particular the oxidosqualene cyclase is an enzyme according to QsbAS (SEQ ID NO 18). The oxidosqualene cyclase is encoded by a nucleotide of SEQ ID NO 17.

The molecule comprising the β-amyrin scaffold is further oxidised to a carboxylic acid, alcohol and aldehyde at the C-28, C-16a and C-23 positions respectively. Another step of this feature of the invention is the oxidation of the molecule comprising the β-amyrin scaffold to form a carboxylic acid at the C-28 position. This step is carried out by a cytochrome P450 monooxygenase. The cytochrome P450 monooxygenase is a C-28 oxidase QsCYP716-C-28 (SEQ ID NO 20). QsCYP716-C-28 is encoded by a nucleotide of SEQ ID NO 19.

Another step of the method of the invention is the oxidation of the molecule comprising the β-amyrin scaffold to form an alcohol at the C-16 position. This step is performed by a cytochrome P450 monooxygenase. The cytochrome P450 monooxygenase is a C-16a oxidase QsCYP716-C-16a (SEQ ID NO 22). QsCYP716-C-16a is encoded by a nucleotide of SEQ ID NO 21.

A further step of the method of the invention is the oxidation of the molecule comprising the β-amyrin scaffold to form an aldehyde at the C-23 position. This step is performed by a cytochrome P450 monooxygenase. The cytochrome P450 monooxygenase is a C-23 oxidase QsCYP714-C-23 (SEQ ID NO 24). QsCYP714-C-23 is encoded by a nucleotide of SEQ ID NO 23.

This feature of the first invention may be in combination with any of the additional features of the first invention mentioned above.

An additional feature of the first aspect of the invention is the chemical synthesis of the QS-21 molecule, starting from QA-Tri(X/R)-FRX(X/A) obtained according to the steps of the first aspect of the invention and including the additional steps of chemically adding the glycosylated C-18 acyl chain, as set out in FIG. 1 and as described in relation to the first aspect of the invention. This feature of the first invention is in combination with one or more of the additional features of the first aspect of the invention mentioned above. This additional feature of the first aspect of the invention may also include combining with the enzyme QsFucSyn (SEQ ID NO 12), as described in the second aspect of the invention. It may also include combining with the enzyme QsFucSyn (SEQ ID NO 12) and the enzyme ATCV-1 (SEQ. ID No 40), or it may include combining with the enzyme ATCV-1 (SEQ ID NO 40) and an enzyme possessing UDP-4-keto-6-deoxy-glucose 4-ketoreductase activity, such as QsFSL-1 (SEQ ID No. 48), QsFSL-2 (SEQ ID No 50), SoFSL-1 (SEQ ID No 52) or SpolFSL (SEQ ID No 54).

This additional feature of the first aspect of the invention may also include combining with the enzyme QsAXS1 (SEQ ID NO 14) as described in the twelfth aspect of the invention.

The thirteenth aspect of the invention is an isolated QA derivative which is QA-TriX/R-F, QA-TriX/R-FR, QA-TriX/R-FRX, QA-TriX/R-FRXX, QA-TriX/R-FRXA, QA-Mono-F, QA-Mono-FR, QA-Mono-FRX, QA-Mono-FRXX, QA-Mono-FRXA, QA-Di-F, QA-Di-FR, QA-Di-FRX, QA-Di-FRXX or QA-Di-FRXA. When the molecule comprises QA-TriX/R-F, QA-TriX/R-FR, QA-TriX/R-FRX, QA-Mono-F, QA-Mono-FR, QA-Mono-FRX, QA-Mono-FRXX, QA-Mono-FRXA, QA-Di-F, QA-Di-FR, QA-Di-FRX, QA-Di-FRXX or QA-Di-FRXA. Said derivatives may also comprise the C-18 acyl chain.

A further aspect of the invention is a QA derivative obtainable or obtained by the method according to the first aspect of the invention and any methods of the invention.

QA derivatives obtained by the method of the invention may be isolated from the biological system. A further aspect of the invention is a method of making a QA derivative comprising the method steps of the invention, including the step of isolating the QA derivative.

Once isolated from the biological system, the QA derivative may be used as an adjuvant to be included in a vaccine composition.

QA derivatives of the present invention may be combined with further immuno-stimulants, such as a TLR4 agonist, in particular lipopolysaccharide TLR4 agonists, such as lipid A derivatives, especially a monophosphoryl lipid A, e.g. 3-de-O-acylated monophosphoryl lipid A (3D-MPL). 3D-MPL is sold under the name ‘MPL’ by GlaxoSmithKline Biologicals N.A. See, for example, U.S. Pat. Nos. 4,436,727; 4,877,611; 4,866,034 and 4,912,094. 3D-MPL can be produced according to the methods described in GB 2 220 211 A. Chemically, it is a mixture of 3-deacylated monophosphoryl lipid A with 4, 5 or 6 acylated chains.

Other TLR4 agonists which may be combined with QA derivatives of the invention include Glucopyranosyl Lipid Adjuvant (GLA) such as described in WO2008/153541 or WO2009/143457 or literature articles (Coler et al. 2011 and Arias et al. 2012).

Adjuvants of the invention may also be formulated into a suitable carrier, such as an emulsion (e.g. an oil-in-water emulsion) or liposomes, as described below.

### Liposomes

The term liposome’ is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material, and/or amphiphilic compounds. A method for making such liposomes is described in WO2013/041572.

Liposome size may vary from 30 nm to several um depending on the phospholipid composition and the method used for their preparation.

The liposome size will be in the range of 50 nm to 200 nm, especially 60 nm to 180 nm, such as 70-165 nm. Optimally, the liposomes should be stable and have a diameter of −100 nm to allow convenient sterilization by filtration.

Structural integrity of the liposomes may be assessed by methods such as dynamic light scattering (DLS) measuring the size (Z-average diameter, Zav) and polydispersity of the liposomes, or, by electron microscopy for analysis of the structure of the liposomes. The average particle size may be between 95 and 120 nm, and/or, the polydispersity (Pdl) index may not be more than 0.3 (such as not more than 0.2).

### EXAMPLES

The present invention is described with reference to the following, non-limiting examples:

### Example 1—Identifying Quillaic Acid C-28 Glycosyltransferase Candidate Genes

We generated Q. saponaria genome sequence data and RNA-seq data for six Q. saponaria tissues (stems, roots, and leaves at four developmental stages: primordia/young leaf/mature leaf/old leaf). This RNA-seq dataset was used to annotate the Q. saponaria genome sequence (Earlham Institute, Norwich, Norfolk). To identify possible biosynthetic gene clusters (BGCs) in the Q. saponaria genome, we used PlantiSMASH, an online platform that automates the identification of candidate plant BGCs (Kautsar et al., 2017). This identified a number of putative BGCs. Many of these clusters were predicted to be involved in saccharide biosynthesis and contained Family 1 UDP-dependent glycosyltransferases (UGTs), a class of enzymes that is almost ubiquitously involved in the glycosylation of plant specialised metabolites.

The biosynthetic genes involved in the biosynthesis of QA-Tri(X/R) predominantly shared an expression profile consisting of high expression in the leaf primordia, low expression in old leaf, and intermediate levels in other tissues. To identify quillaic acid C-28 glycosyltransferase candidate, we carried out a co-expression analysis using a self-organising map (SOM). For the identification of new candidates, the four genes required for the biosynthesis of quillaic acid (QA) (QsbAS, and the C-28, C-23 and C-16a oxidases) were used as baits. Transcripts were prioritised based on how often they were identified as being co-expressed with any of these bait genes. This identified multiple UGT enzymes as potential candidates but did not identify likely glycosyltransferase gene candidates in unusual enzyme classes.

The previously identified QS-21 biosynthetic enzymes are expressed at high levels in primordia. We carried out a search for UGT candidates that were well expressed in primordia in order to identify candidates that may not be strictly co-expressed but that have an overlapping expression profile. Out of the Q. saponaria genomic sequences that were annotated as encoding UGTs, we selected sequences that had an RNA-seq expression value of at least 30 FPKM in the primordia tissue. We excluded sequences that were less than 400 amino acids in length and carried out a phylogenetic analysis of the predicted amino acid sequences of the resulting sequences.

In order to clone candidate genes, a series of oligonucleotide primers were designed which incorporated 5′ attB sites upstream of the target sequence to allow for Gateway® cloning. Using these primers, genes were amplified by PCR from Q. saponaria leaf cDNA and cloned into pDONR 207. The clones were sequenced before transfer into the plant expression vector pEAQ-HT-DEST1 (Sainsbury et al., 2009). The expression constructs were then transformed individually into Agrobacterium tumefaciens (LBA4404) for transient expression in N. benthamiana.

### Example 2—Identification of Quillaic Acid 28-O-Fucose-Ester-Transferase (Qs-28-O-FucT)

The C-28 linear tetrasaccharide is initiated with a D-fucose attached by an ester linkage to the C-28 position of the quillaic acid scaffold. In our shortlisting of potential C-28 glycosyltransferase candidates, we identified two fucosyltransferase enzyme candidates (Ross et al, 2011 and Sasaki et al, 2014). One of these was not identified as being co-expressed with the quillaic acid biosynthetic genes or within a biosynthetic gene cluster. In contrast, one was co-expressed with quillaic acid biosynthetic genes, within a BGC, and it was more closely related to a known triterpene carboxylic acid glucosyltransferase.

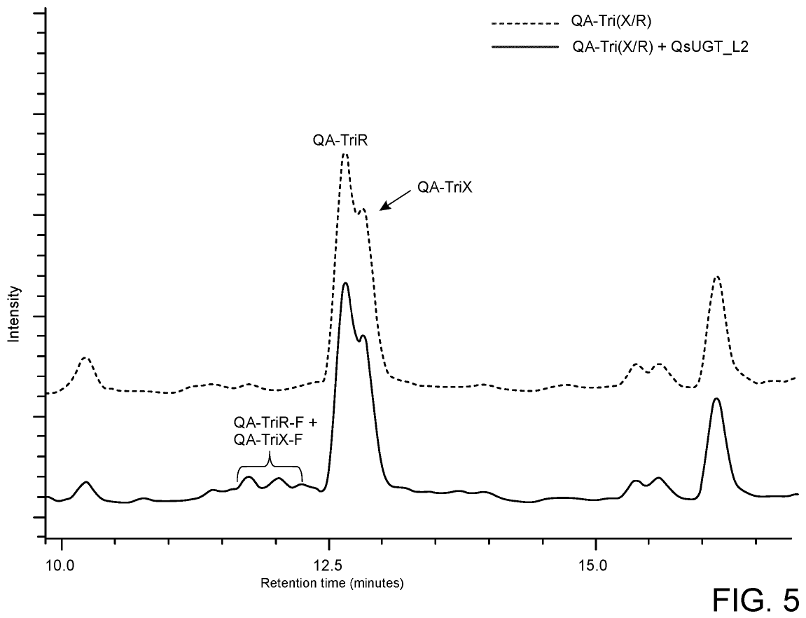

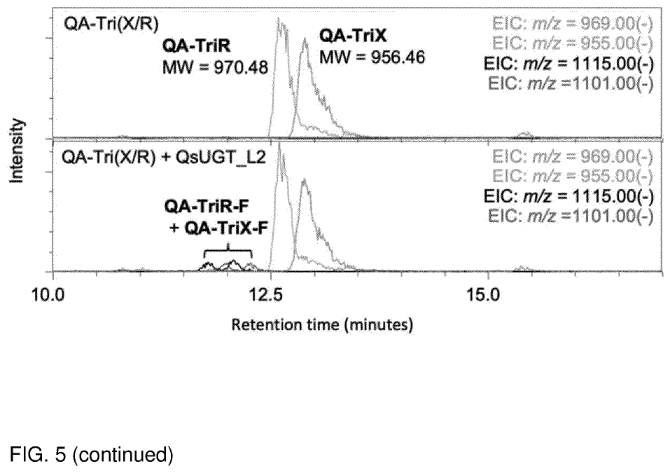

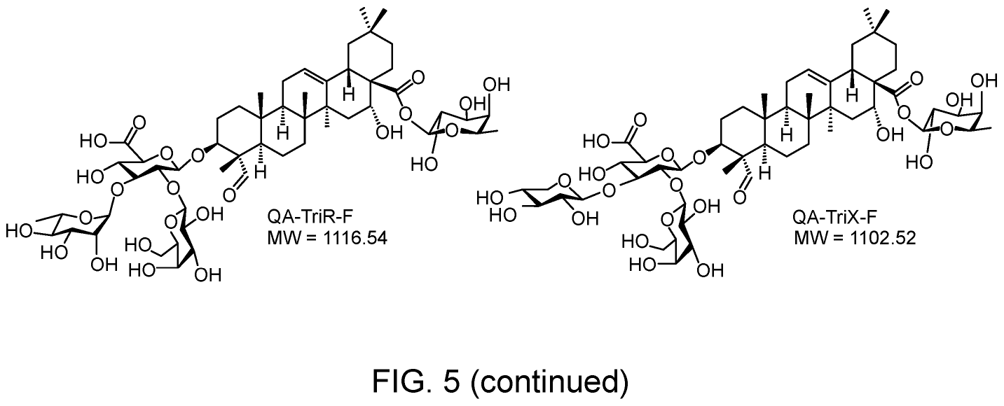

To screen the fucosyltransferase enzyme candidates for activity, we transiently co-expressed the gene sets required for production of QA-Tri(X/R) (both Xylp and Rhap versions of the quillaic acid C-3 trisaccharide) in N. benthamiana leaves. In addition, a truncated, feedback-insensitive form of the Avena strigosa HMG-CoA reductase (AstHMGR) was also included, as this has previously been shown to increase the production of triterpenes produced in N. benthamiana. Finally, the fucosyltransferase enzyme candidates were transiently co-expressed with the above genes. Further details are provided earlier in the text when discussing the method step of attaching D-fucose with a β-linkage to a molecule comprising QA as well as under Materials and Methods, HPLC-CAD-MS analysis of infiltrated leaf extracts revealed that QsUGT_L2 had activity consistent with the addition of a sugar with the mass of a d-fucose to both QA-TriX and QA-TriR to form QA-TriX-F and QA-TriR-F, respectively (FIG. 5). QsUGT_L1 showed no activity. Therefore, QsUGT_L2 was renamed Qs-28-O-FucT. The product yields of this enzyme were very low, anticipated to be due to the necessary sugar nucleotide cofactor (UDP-α-D-fucose) being limited in N. benthamiana (see Example 6—Optimising UDP-fucose availability in N. benthamiana section (Example 6). Consequently, the QA-Tri(X/R) remained the predominant products in the extracts (FIG. 5).

### Example 3—Identification of Quillaic Acid 28-O-Fucoside [1,2]-Rhamnosyltransferase (Qs-28-O-RhaT)

In order to identify the second C-28 glycosyltransferase, UGT candidates were transiently co-expressed in N. benthamiana leaves with the genes required to produce QA-TriX-F. Further details are provided earlier in the text when discussing the method step of attaching α-L-rhamnose to a β-D-fucose residue as well as under Materials and Methods.

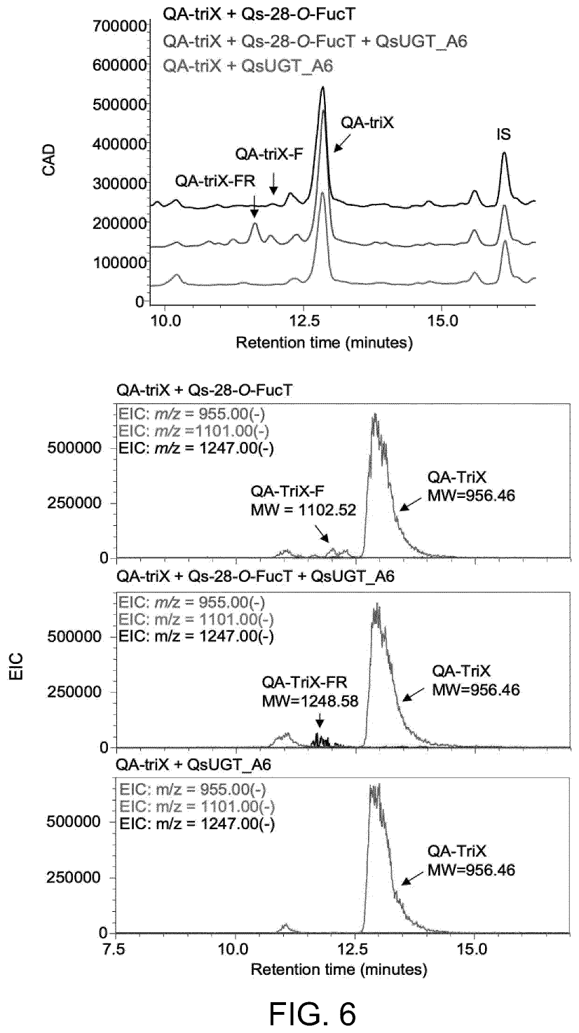

Analysis by HPLC-CAD-MS showed that the addition of one candidate, QsUGT_A6, resulted in the complete reduction of the QA-TriX-F peak and the appearance of a new more polar peak at 11.6 minutes with a mass consistent with the addition of a rhamnose sugar to QA-TriX-F (FIG. 6). Activity was not seen in the absence of Qs-28-O-FucT, suggesting that the activity of QsUGT_A6 is dependent on the fucosyltransferase activity of Qs-28-O-FucT (6). QsUGT_A6 was identified as a candidate due to high expression in primordia as is seen for the genes required to make quillaic acid, and additionally QsUGT_A6 was identified in the same BGC, Cluster 50, as CSL1 and Qs-28-O-FucT. QsUGT_A6 was therefore referred to Qs-28-O-RhaT

### Example 4—Identification of Quillaic Acid 28-O-Fucoside [1,2]-Rhamnoside [1,4] Xylosyltransferase (Qs-28-O-XylT3)

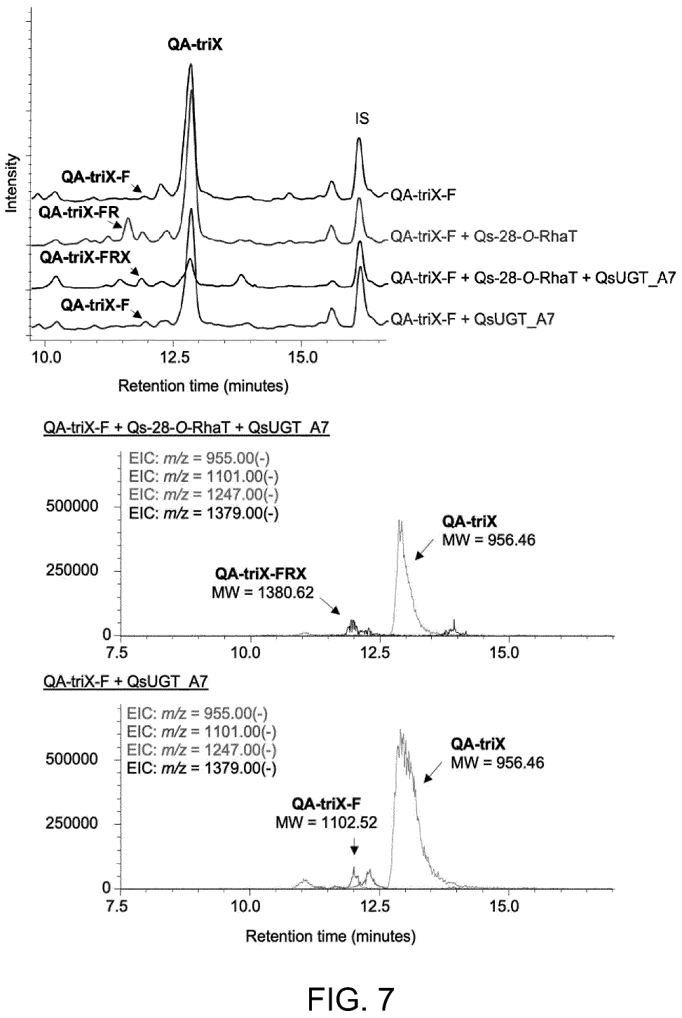

To search for the glycosyltransferase that adds the third sugar in the C-28 sugar chain, UGT candidates were screened for activity by transient co-expression in N. benthamiana with the genes required to make QA-TriX-FR. Further details are provided earlier in the text when discussing the method step of attaching a β-D-xylose to a α-L-rhamnose residue as well as under Materials and Methods. This revealed that the addition of one candidate, QsUGT_A7, resulted in the consumption of the QA-TriX-FR peak and the appearance of a less polar peak which had a mass consistent with the addition of a xylose to QA-TriX-FR (FIG. 7). The activity of QsUGT_A7 was dependent on the activity of Qs-28-O-RhaT, as without Qs-28-O-RhaT, QsUGT_A7 did not glycosylate QA-TriX-F (7). As QsUGT_A7 adds the xylose that is the third sugar in the C-28 sugar chain, this enzyme is referred to as Qs-28-O-XylT3.

### Example 5—Identification of a quillaic acid 28-O-fucoside [1,2]-rhamnoside [1,4] xyloside [1,3] xylosyltransferase and a quillaic acid 28-O-fucoside [1,2]-rhamnoside [1,4] xyloside [1,3] apiosyltransferase

At this stage, issues with UDP-α-D-fucose availability resulted in the production of very small amounts of C-28 glycosylated products in N. benthamiana leaves (FIG. 7). Consequently, this made it difficult to carry out a screen for the enzyme(s) involved in the fourth step in the C-28 sugar chain, as it was unclear whether any new products would be produced in sufficient quantities to be detected. To circumvent this, we attempted to substitute the C-28 D-fucose with the more abundant D-glucose, as both sugars possess the same C-2 hydroxyl group configuration to which the subsequent C-28 L-rhamnose is attached. To achieve this, the C-28 glucosyltransferase CaUGT73AD1 from Centella asiatica was tested as a replacement for the Qs-28-O-FucT (de Costa et al, 2017).

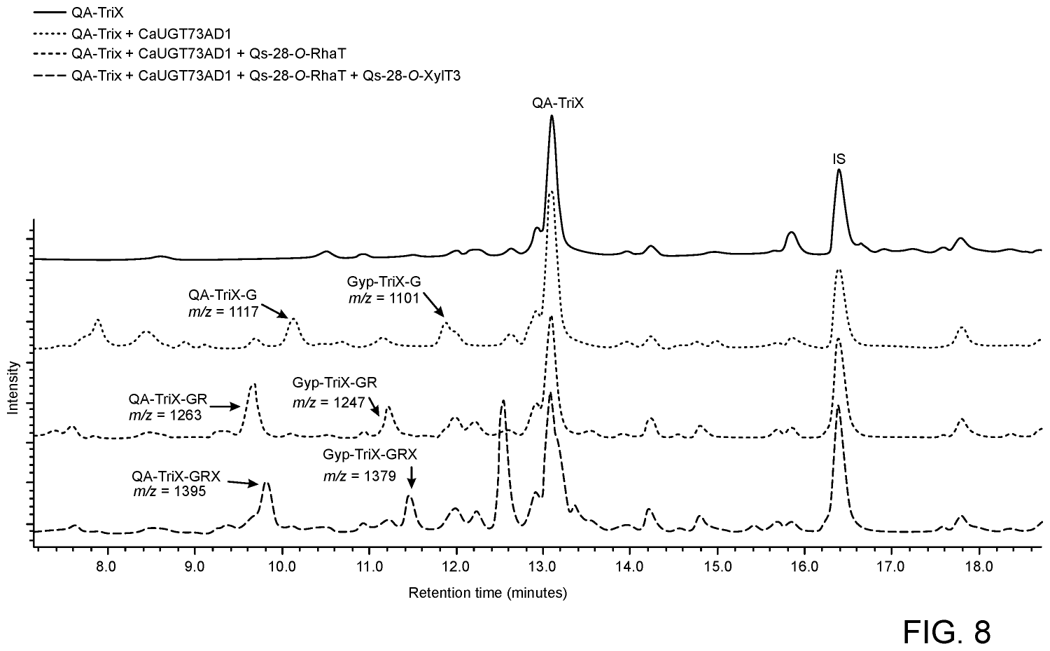

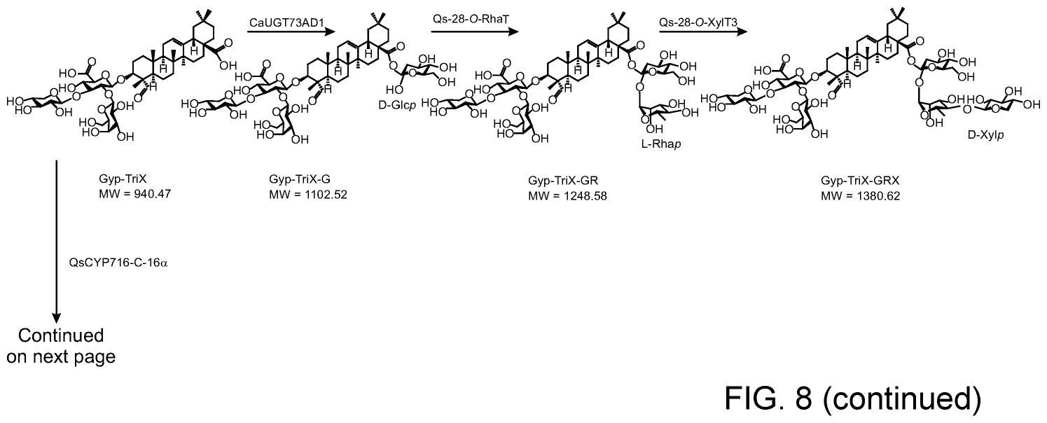

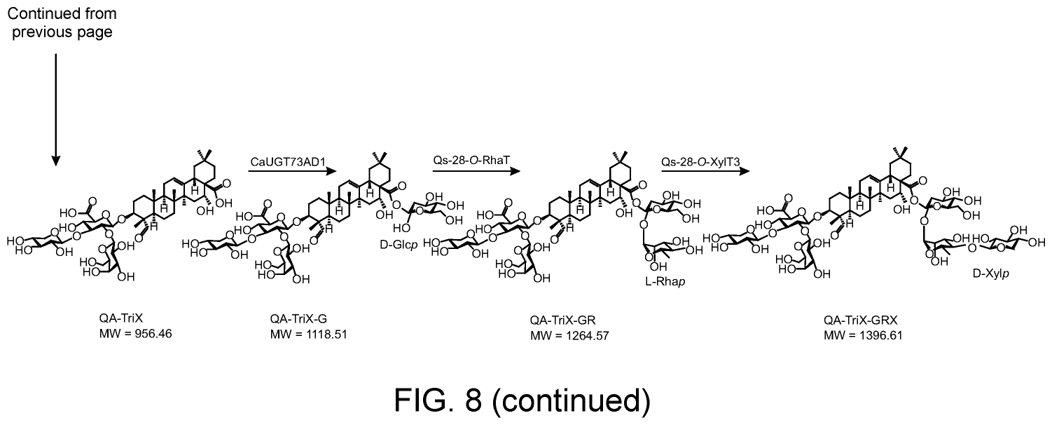

We transiently co-expressed CaUGT73AD1 in N. benthamiana leaves with the genes required for the production of QA-TriX. HPLC-CAD-MS analysis of leaf extracts showed that the addition of CaUGT73AD1 resulted in the appearance of a new peak at 10.1 minutes, with a mass ion (m/z=1117) consistent with the addition of a glucose to QA-TriX to form QA-TriX-G (MW=1118.51) (FIG. 8). In addition, there was also a new peak at 11.8 minutes with an m/z of 1101. It has been previously observed that the conversion of the triterpene scaffold from gypsogenin (Gyp) to quillaic acid by QsCYP716-C-16a is not always complete in the N. benthamiana system (WO 2019/122259). This results in an accumulation of glycosylated intermediates with a gypsogenin scaffold in place of quillaic acid. The new peak at 11.8 minutes is consistent with the addition of a glucose by CaUGT73AD1 to the gypsogenin trisaccharide Gyp-TriX, to form Gyp-TriX-G (MW=1102.52) (FIG. 8).

We then tested whether Qs-28-O-RhaT and Qs-28-O-XylT3 could utilise the CaUGT73AD1 products. Further details are provided earlier in the text when discussing the method step of attaching β-D-xylose to a β-D-xylose residue or the method step of attaching β-D-apiose to a β-D-xylose residue as well as under Materials and Methods. The addition of Qs-28-O-RhaT resulted in a reduction of the QA-TriX-G and Gyp-TriX-G peaks at 10.1 and 11.8 minutes, and the appearance of two new more polar peaks at 9.5 minutes (m/z=1263) and 11.1 minutes (m/z=1247) which are consistent with the addition of a rhamnose to QA-TriX-G and Gyp-TriX-G, respectively (FIG. 8). The further addition of Qs-28-O-XylT3 resulted in the reduction of the QA-TriX-GR and Gyp-TriX-GR peaks, and the appearance of peaks at 9.8 minutes (m/z=1395) and 11.5 minutes (m/z=1379) which are consistent with the further addition of a xylose (FIG. 8). This suggests that Qs-28-O-RhaT and Qs-28-O-XylT3 are able to utilise a triterpene glycoside substrate with a glucose at the C-28 position. The resulting hexasaccharides (QA-TriX-GRX and Gyp-TriX-GRX) are accumulated in sufficient amounts to allow a screen for the fourth C-28 glycosyltransferase(s).

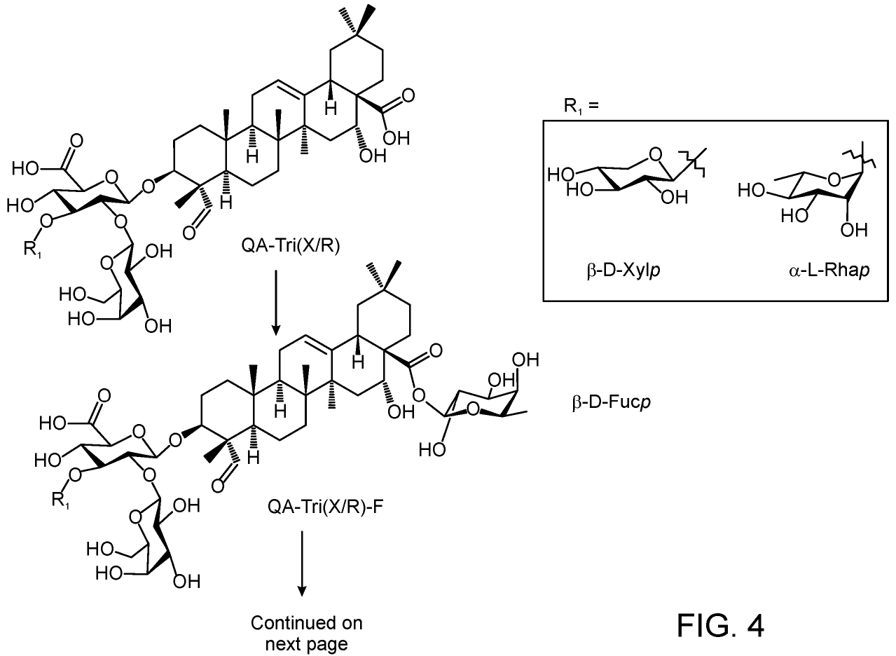

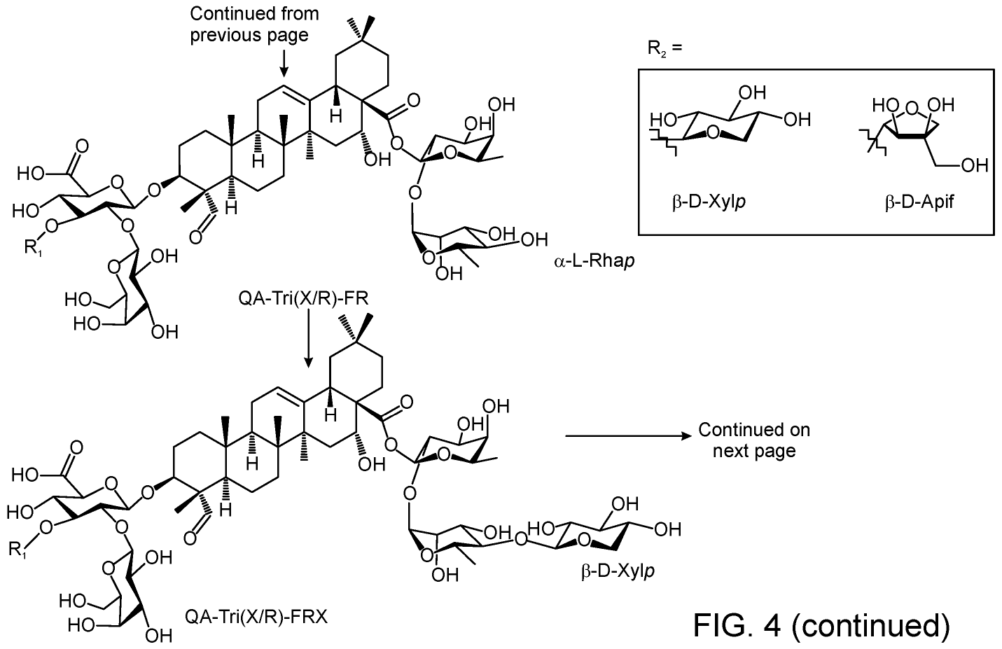

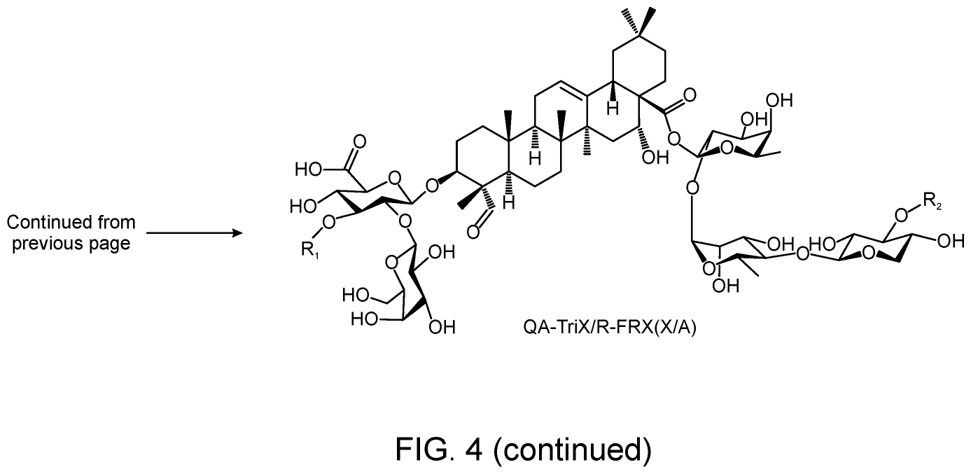

The final step in the C-28 sugar chain is the addition of a D-xylose or a D-apiose (FIG. 4). In our experiments, UDP-α-D-xylose has not been found to be limiting in N. benthamiana. However, as for UDP-α-D-fucose, we considered potential low levels of UDP-α-D-apiose in N. benthamiana as a potential bottleneck in identifying the QS-21 apiosyltransferase.

D-Apiose is found in the pectic polysaccharide rhamnogalacturonan II (RG-II) in the cell walls of higher plants and plays a crucial role in the formation of cross-links in plant cell walls. UDP-α-D-apiose is synthesized from UDP-α-D-glucuronic acid by bifunctional enzymes, UDP-apiose/UDP-xylose synthases (AXSs), that also produce UDP-α-D-xylose. In Nicotiana benthamiana, this activity is carried out by NbAXS1. VIGS silencing of NbAXS1 resulted in growth defects and cell death likely due to deficiencies in the apiose-containing side chains of RG-II. The levels of UDP-α-D-xylose were not affected by the silencing of NbAXS1, as UDP-α-D-xylose is predominantly synthesized by UDP-D-glucoronate decarboxylases in higher plants.

The ratio of UDP-α-D-apiose and UDP-α-D-xylose produced by different AXSs can vary: a higher amount of UDP-α-D-xylose is produced by NbAXS1 and AtAXS1 in N. benthamiana and A. thaliana, whilst UDP-α-D-apiose is produced predominantly in the case of AXSs from parsley and duckweed (Lemna minor), plants that contain D-apiose in abundance in the secondary metabolite apiin and the pectic polysaccharide apiogalacturonan. This suggests that increased levels of UDP-α-D-apiose production may have evolved in plants that are rich in apiose, and that there may be insufficient levels of UDP-α-D-apiose in N. benthamiana for the heterologous production of D-apiose-containing secondary metabolites such as QS-21.

The self-organising map co-expression analysis of Q. saponaria genes identified an ‘UDP-D-apiose/UDP-D-xylose synthase 2’ (QsAXS1) that was co-expressed with the QA genes and highly expressed in the primordia, indicating that this gene may be important in QS-21 biosynthesis. This gene was cloned from Q. saponaria leaf cDNA for co-expression in N. benthamiana.

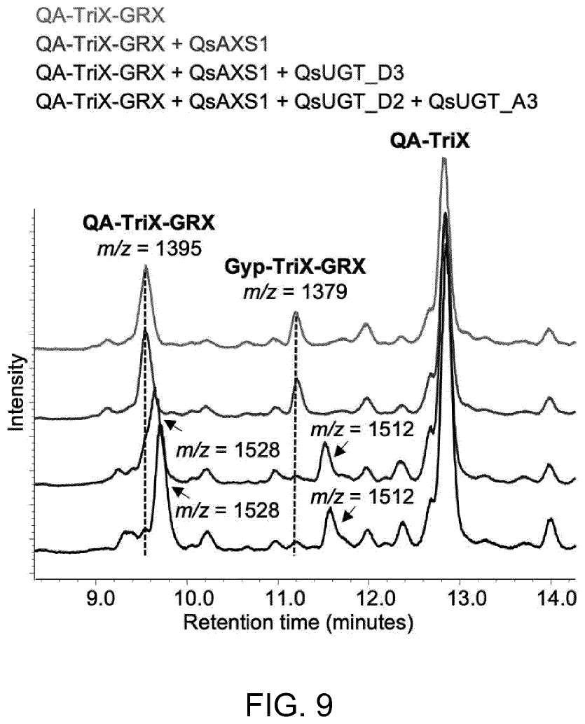

Co-expression of QsAXS1 with the genes required for the production of QA-TriX-GRX and Gyp-TriX-GRX did not affect the accumulation of these products (FIG. 9). QsAXS1 was included in the screen for the fourth C-28 sugar transferases using QA-TriX-GRX and Gyp-TriX-GRX as substrates. Two combinations of candidate UGTs within the screen altered the compounds accumulated. The first combination was the addition of QsUGT_D3, which resulted in the reduction of the substrate peaks (QA-TriX-GRX, 9.5 minutes, m/z=1395 and Gyp-TriX-GRX, 11.2 minutes, m/z=1379), and the accumulation of two new peaks at 9.6 minutes (m/z=1528) and 11.5 minutes (m/z=1512) (FIG. 9). The second combination was the addition of two candidates, QsUGT_D2 and QsUGT_A3, which also resulted in the reduction of the QA-TriX-GRX and Gyp-TriX-GRX substrate peaks and the appearance of new peaks at 9.7 minutes (m/z=1528) and 11.6 minutes (m/z=1512) (FIG. 9). The masses of the new peaks accumulated in these combinations are consistent with the addition of a pentose, such as apiose or xylose, to QA-TriX-GRX and Gyp-TriX-GRX to form QA-TriX-GRX(X/A) (MW=1528.66) and Gyp-TriX-GRX(X/A) (MW=1512.66), respectively.

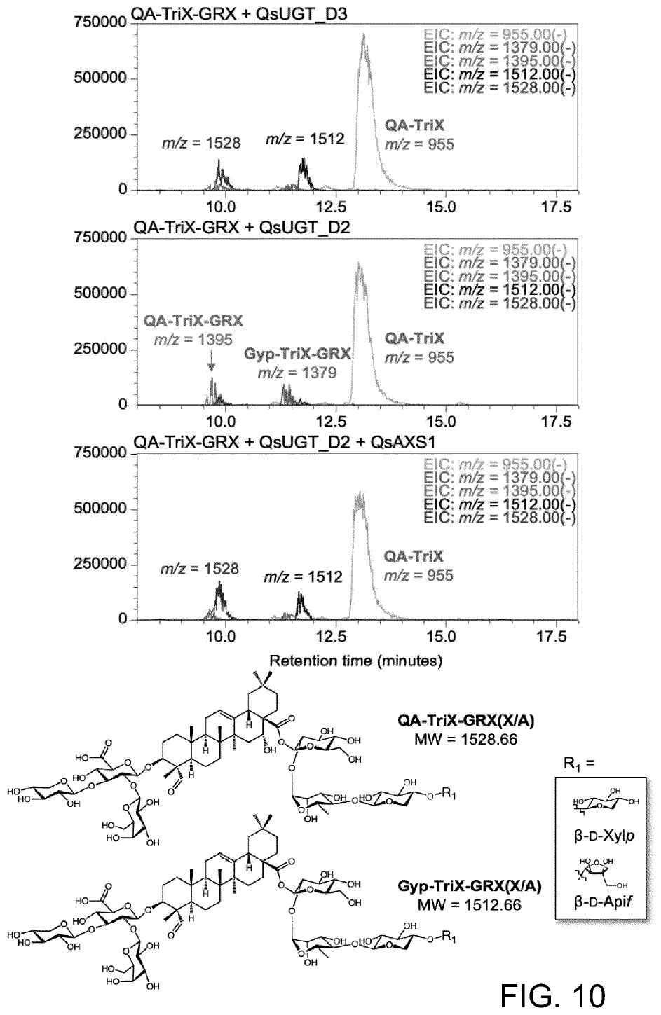

We tested whether the addition of QsAXS1 was necessary for any of the observed activities. QsUGT_D3 showed activity in the absence of QsAXS1, suggesting that this enzyme is not dependent on QsAXS1 activity (FIG. 10). As QsUGT_A3 and QsUGT_D2 had been screened together, we tested these two enzymes separately to determine which enzyme was responsible for the previously observed activity. QsUGT_A3 did not show any activity when tested in the presence or absence of QsAXS1, so we concluded that this enzyme is not involved in this pathway step. QsUGT_D2 was active in the presence of QsAXS1, confirming this enzyme as a candidate (FIG. 10). In the absence of QsAXS1, QsUGT_D2 showed a large reduction in activity, converting very little of the precursors QA-TriX-GRX and Gyp-TriX-GRX (FIG. 10).

These results suggest that QsUGT_D3 is the quillaic acid 28-O-fucoside [1,2]-rhamnoside [1,4] xyloside [1,3] xylosyltransferase, as it was not dependent on the activity of QsAXS1. UDP-α-d-xylose is predominantly produced by UDP-D-glucoronate decarboxylases, and the activity of AXSs are not expected to significantly contribute to the available pool of UDP-α-d-xylose present in N. benthamiana. It is therefore unlikely that the addition of QsAXS1 would affect the activity of a xylosyltransferase. We subsequently referred to QsUGT_D3 as Qs-28-O-XylT4.

The activity of QsUGT_D2 was dependent on co-expression with QsAXS1. This suggests that QsUGT_D2 is an apiosyltransferase, as co-expressing QsAXS1 may be expected to affect the levels of UDP-α-D-apiose available in N. benthamiana. We therefore referred to QsUGT_D2 as Qs-28-O-ApiT4. This result also indicates that whilst UDP-α-D-apiose is known to be present in N. benthamiana due to its roles in primary metabolism, the level of UDP-α-D-apiose produced by the endogenous NbAXS1 is not sufficient for the heterologous production of D-apiose-containing secondary metabolites in N. benthamiana. When a heterologous host is limited in the availability of UDP-α-D-apiose, but produces sufficient levels of UDP-α-D-glucuronic acid (such as N. benthamiana), co-expression with QsAXS1 can increase the availability of UDP-α-D-apiose by the conversion of UDP-α-D-glucuronic acid to UDP-α-D-apiose.

### Example 6—Optimising UDP-Fucose Availability in N. benthamiana

Part A: Infiltration of D-Fucose Results in Production of UDP-D-Fucose in N. benthamiana

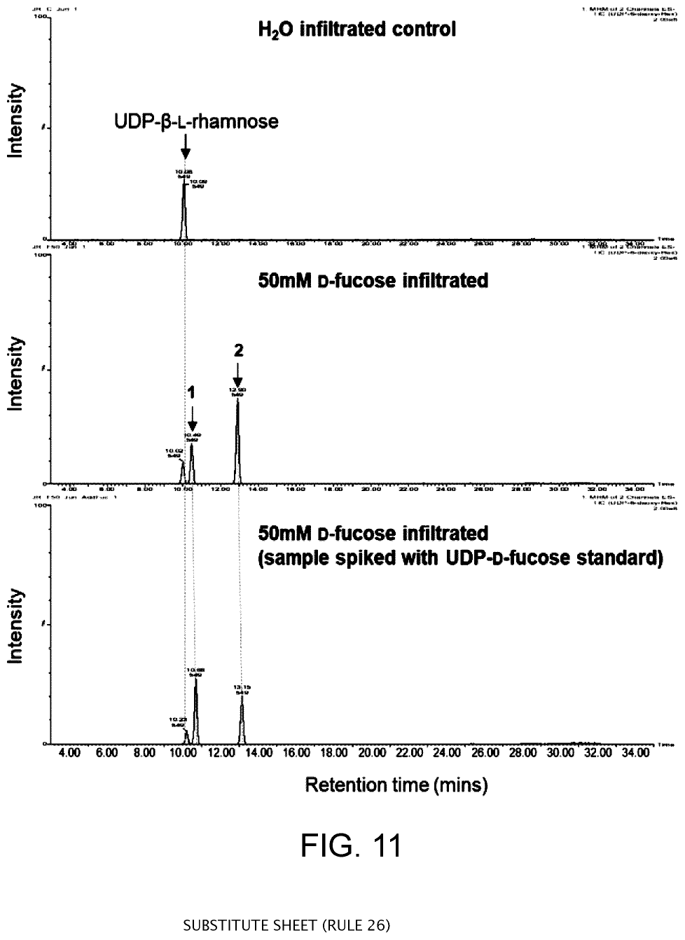

The activated form of D-fucose occurring in plants is anticipated to be UDP-α-D-fucose based on previous studies in foxglove (Faust et al, 1994). Furthermore, the fucosyltransferase Qs-28-O-FucT is a UGT, which are known to require UDP-sugars as cofactors. The relatively poor accumulation of the fucosylated compounds suggested that the relevant sugar nucleotide (anticipated to be UDP-α-D-fucose) was significantly limiting in N. benthamiana. Therefore, strategies for boosting UDP-α-D-fucose were considered. As a first strategy, exogeneous supplementation with the free monosaccharide (D-fucose) was performed to determine whether the sugar could be taken up by the cells and utilised with the sugar salvage pathway to convert D-fucose to UDP-α-D-fucose. Therefore, solutions of D-fucose (50 mM, plus a water-only control) were infiltrated using a needleless syringe into N. benthamiana leaves. Leaves were harvested after three days and sugar nucleotide profiling was performed. LC-MS/MS analysis determined that only a single UDP-deoxyhexose could be detected in control (water infiltrated) extracts, corresponding to UDP-β-L-rhamnose. By contrast, two new UDP-deoxyhexose products could be detected in the D-fucose-infiltrated leaves (FIG. 11). In order to determine the presence of UDP-α-D-fucose unequivocally in the plant extracts, a standard of UDP-α-D-fucose was first synthesised using an enzymatic synthesis method as previously reported (Errey et al., 2004). This standard was spiked into the 50 mM D-fucose-infiltrated leaf extract and found to coelute perfectly with the first of the new peaks, thus confirming the presence of UDP-α-D-fucose in N. benthamiana. The identity of the second peak was not determined but is anticipated to be UDP-α-D-quinovose (the C-4 epimer of UDP-α-D-fucose) due to the action of endogenous C-4 epimerases present in N. benthamiana.

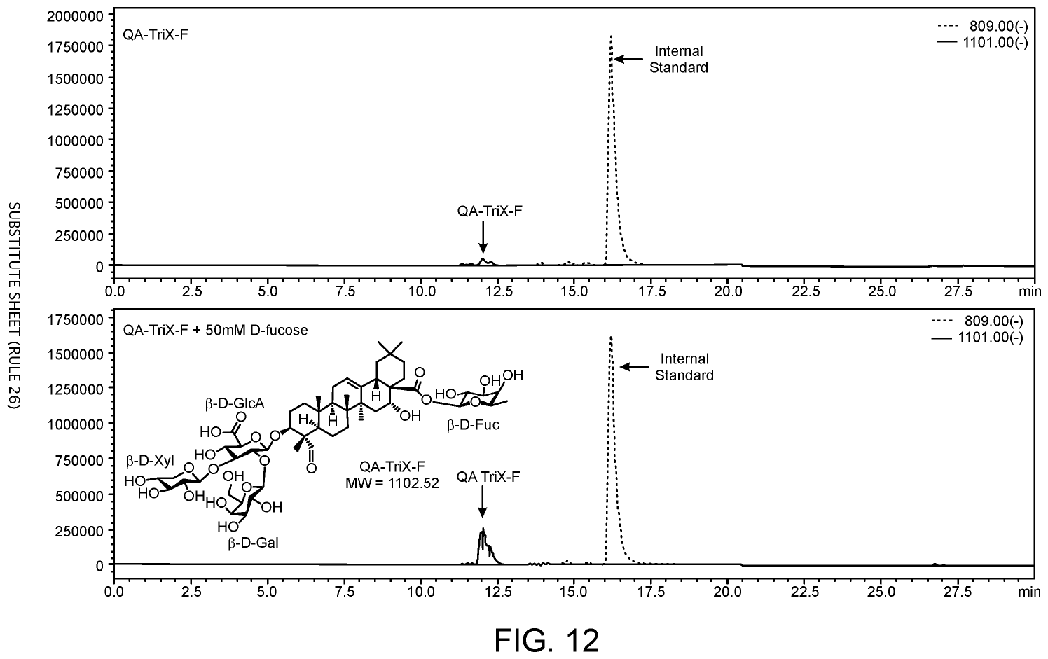

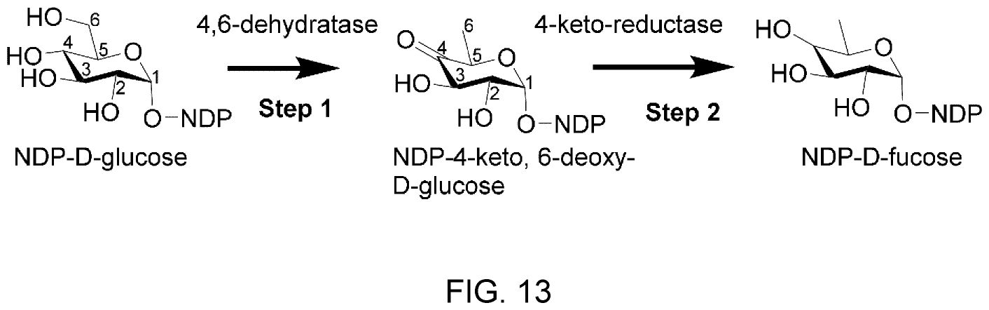

Following the confirmation that UDP-α-D-fucose levels can be boosted in planta by infiltration of the free D-fucose monosaccharide, the next experiment sought to determine whether increased abundance of UDP-α-D-fucose improved levels of the fucosylated triterpene. The genes necessary for production of the fucosylated QA-TriX product (QA-TriX-F) were transiently expressed by agroinfiltration of N. benthamiana. 50 mM D-fucose was included in the infiltration buffer to boost the UDP-α-D-fucose content. LC-MS analysis of leaf extracts revealed a significant increase in the abundance of the QA-TriX-F product in leaves infiltrated with 50 mM D-fucose compared to buffer-only controls (FIG. 12). This therefore demonstrates that higher production of the fucosylated saponin QA-TriX-F can be achieved by increasing the abundance of UDP-α-D-fucose.

Part B: Expression of NDP-D-Fucose Biosynthetic Enzymes from Non-Plant Species

The cost of D-fucose would make infiltration of this sugar uneconomical for large-scale production of saponins. Consequently, it would be preferable to engineer production of D-fucose in N. benthamiana from endogenous sugar nucleotide pools. Although no D-fucose biosynthetic pathway is known in plants, based on examples from other organisms, the most likely route for biosynthesis of NDP-D-fucose is a two-step process starting from NDP-D-glucose. The first step involves conversion of NDP-D-glucose to an NDP-4-keto-6-deoxy glucose intermediate, catalysed by an NDP-D-glucose 4,6-dehydratase. The second step is formation of NDP-D-fucose from NDP-4-keto-6-deoxy glucose by stereoselective reduction of the C-4 keto group to an axial hydroxyl group, catalysed by a 4-ketoreductase (FCD) (Figure).

We therefore attempted to identify and transiently express previously characterised enzymes which could carry out these two activities and determine their effect on yield of fucosylated saponins in N. benthamiana. The first of these two steps is common to both NDP-D-fucose and NDP-L-rhamnose biosynthesis and hence the 4-keto-6-deoxy glucose intermediate should be produced in N. benthamiana. However 4,6-dehydratase is not found as a discrete enzyme in higher plants, but rather as part of a larger rhamnose synthase (RHM), in which 4,6-dehydratase, 3,5-epimerase and 4-keto-reductase are present in a single enzyme. Therefore, we chose a UDP-D-glucose 4,6-dehydratase from the Acanthocystis turfacea chlorella virus 1 (ATCV-1), which is known to produce UDP-4-keto-6-deoxy glucose from UDP-D-glucose. For the second FCD step, the only known enzymes are from D-fucose-producing bacteria, including Aggregatibacter actinomycetemcomitans, Anoxybacillus tepidamans, Escherichia coli and Streptomyces griseoflavus. These bacterial enzymes are anticipated to utilise dTDP-sugars rather than UDP sugars as observed in plants. Therefore, to enhance the chance of identifying a functional enzyme, the FCD enzymes from A. actinomycetemcomitans, A. tepidamans and E. coli (AaFCD, AtFCD and EcFCD, respectively) were chosen for transient expression.

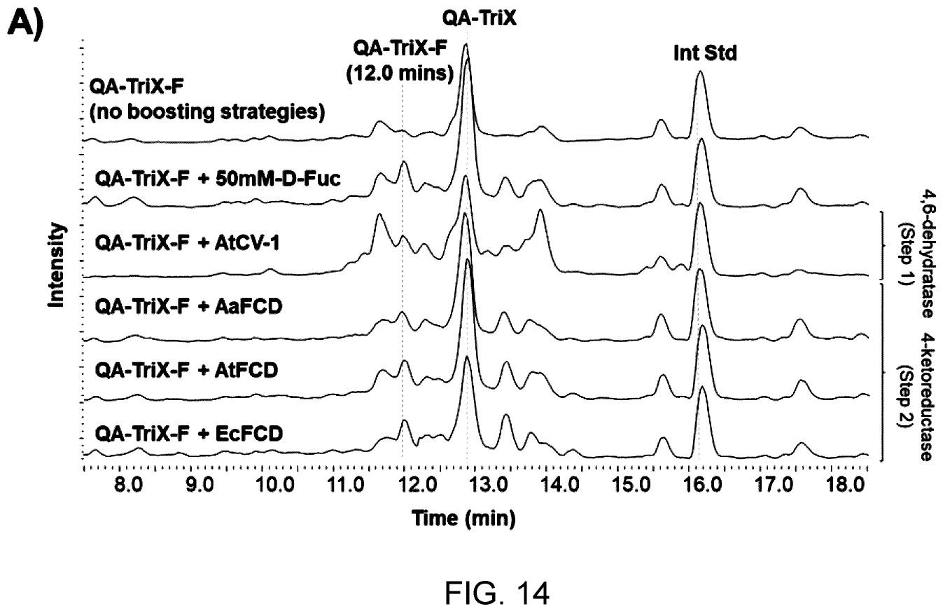

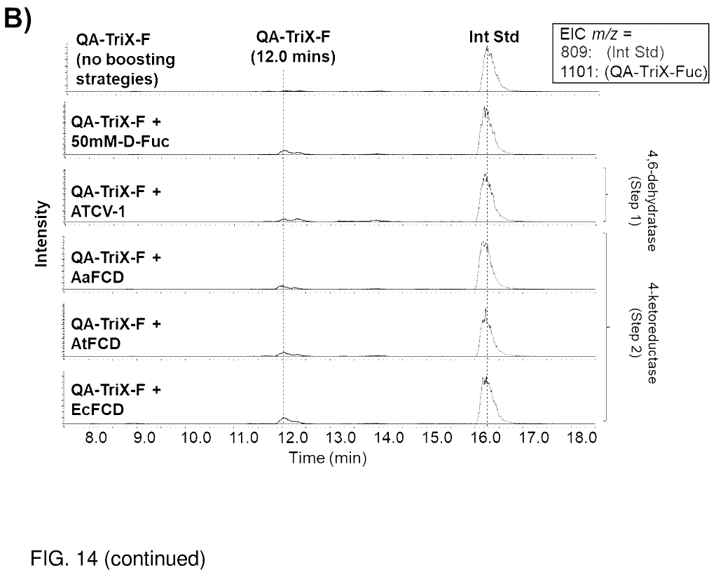

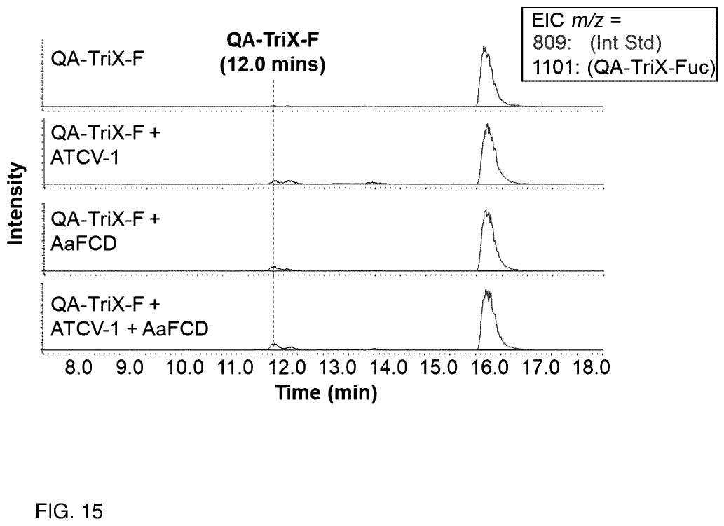

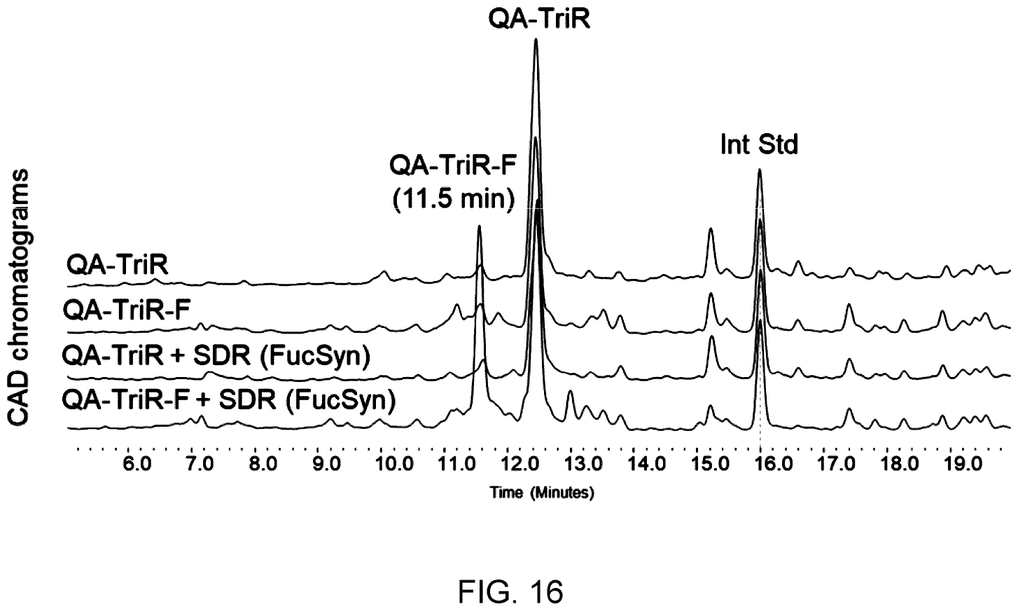

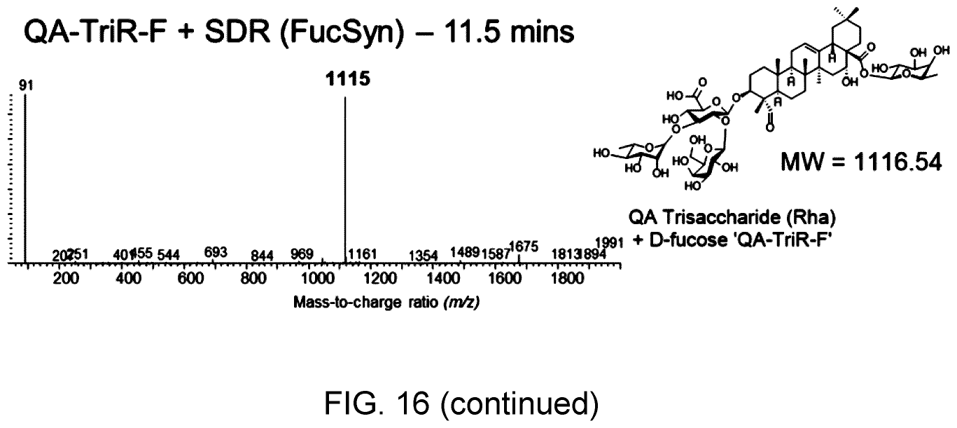

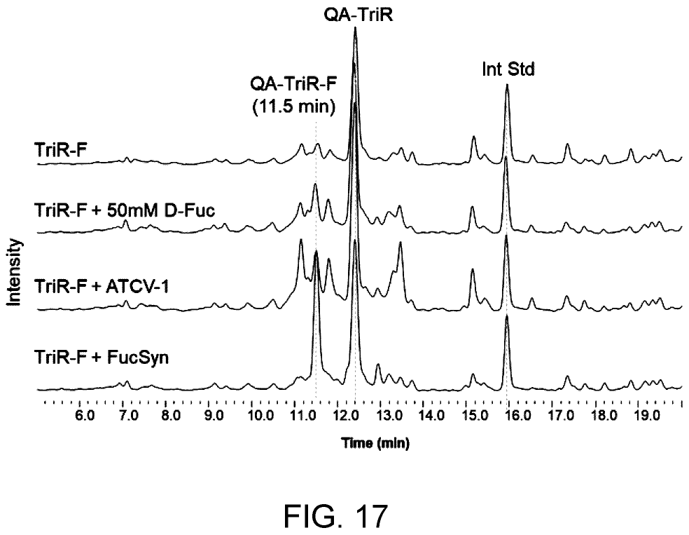

Each of the 4 enzymes (ATCV-1 and the three FCD genes) were transiently expressed in N. benthamiana alongside the gene set necessary for production of the QA-TriX-F product (AstHMGR, QsbAS, QsCYP716-C-28+QsCYP716-C-16α+QsCYP714-C-23+QsCSL1+Qs-3-O-Ga/T+Qs_0283870+Qs-28-O-FucT). We observed that each of the enzymes was capable of providing a small boost to the QA-TriX-F product compared to controls, and that the amount accumulated was comparable to that produced by infiltration of 50 mM D-fucose (FIG. 14). These results suggest that UDP-D-fucose biosynthesis is likely to proceed via a similar route in N. benthamiana to that identified in bacteria, and that the ATCV-1 enzyme or FCD enzymes are capable of working with the endogenous metabolites in N. benthamiana to enhance levels of UDP-D-fucose. Finally, the ATCV-1 enzyme was co-expressed with AaFCD to see if product yields could be enhanced further. However this approach seemed to have little effect on QA-TriX-F production over either enzyme individually (FIG. 15). These results demonstrate that it is possible to increase the content of fucosylated saponins in N. benthamiana by co-expression of the UDP-D-glucose 4,6-dehydratase (ATCV-1) or bacterial 4-ketoreductase (FCD) and that it is not necessary to include additional D-fucose.

Part C: Identification of a Fucose-Boosting Enzyme from Q. Saponaria and Purification of the C-28 Qlycosides.

Although both co-infiltration of D-fucose or co-expression of the NDP-D-fucose biosynthetic enzymes resulted in a boost to production of the fucosylated products, the relative conversion of the non-fucosylated precursors (QA-Tri) remained relatively poor (see FIG. 14A), suggesting that UDP-D-fucose may still be limiting and prompting further attempts to investigate D-fucose production. Therefore, an investigation into the possible biosynthesis of UDP-α-D-fucose in Q. saponaria was performed. The previously described BGC cluster ‘50’ contains several genes relevant to QS-21 biosynthesis, including the C-16 oxidase, the QsCSL1 gene (GlcpAT), the Qs28-O-RhaT and the Qs-28-O-FucT, plus several genes of unknown function. Amongst these unknown genes, an oxidoreductase, annotated as a member short chain dehydrogenase/reductase superfamily (SDR), was found to be present. Most of the known sugar nucleotide interconverting enzymes (NSEs), which are responsible for the biosynthesis of the various UDP-sugars found in QS-21 are also members of the SDR superfamily. Therefore, the enzyme was cloned and transiently expressed in N. benthamiana with the suite of genes necessary for production of the QA-TriR-F product (variant with rhamnose within the C-3 trisaccharide). The inclusion of the clustered SDR resulted in a marked increase to the amount of the QA-TriR-F product, suggesting that the SDR is capable of enhancing the activity of the fucosyltransferase. The SDR is therefore henceforth called QsFucSyn. It was necessary to include both the Qs-28-O-FucT and the QsFucSyn enzyme to get the large increase of the QA-TriR-F product.

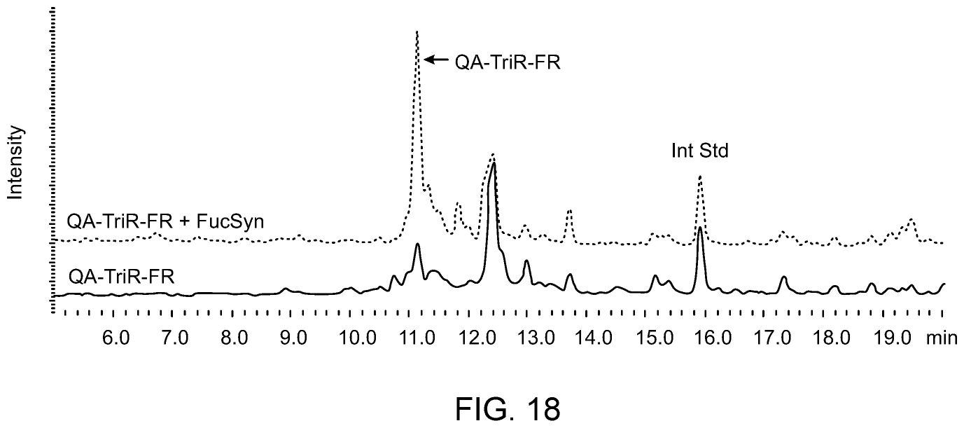

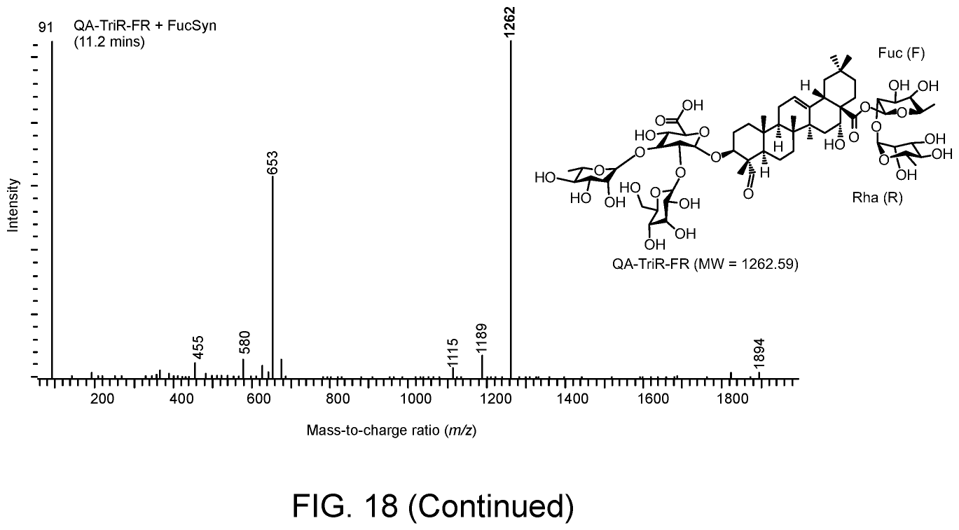

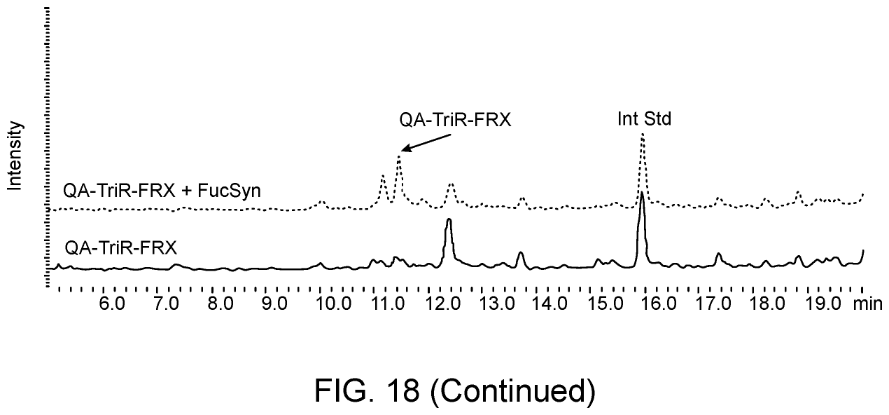

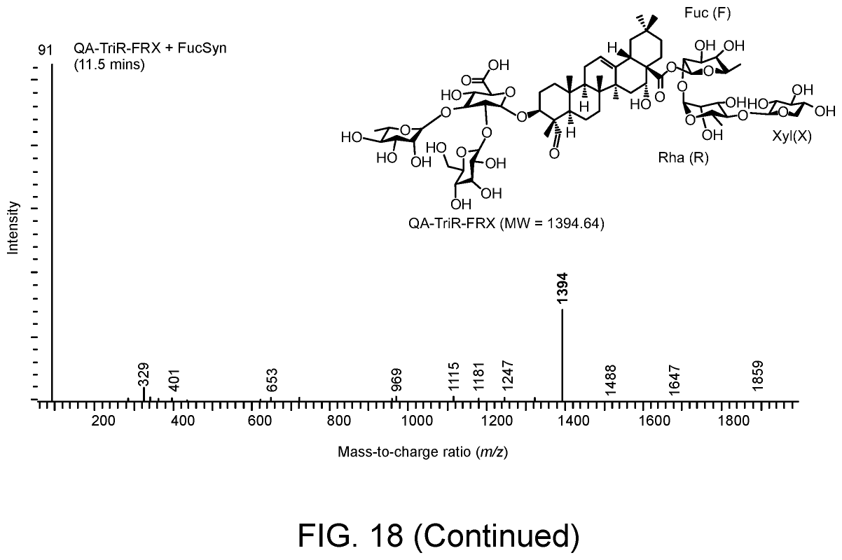

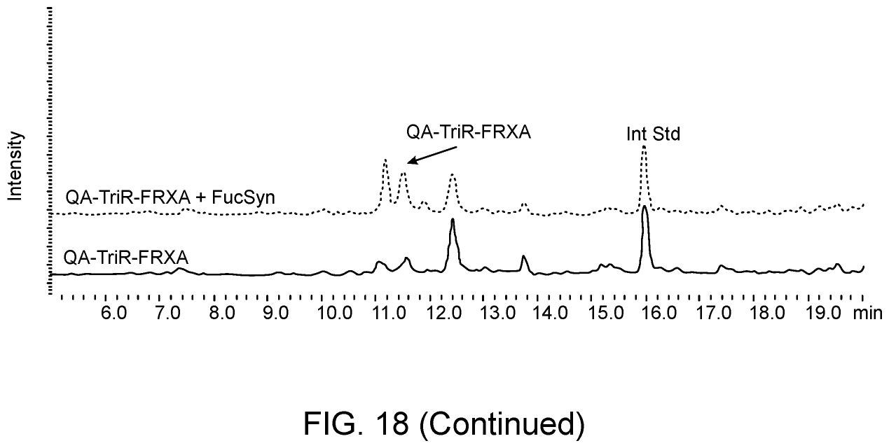

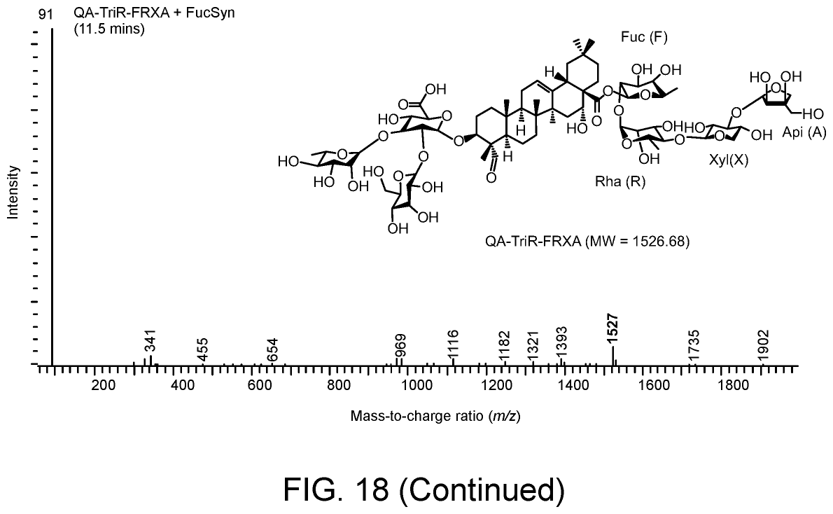

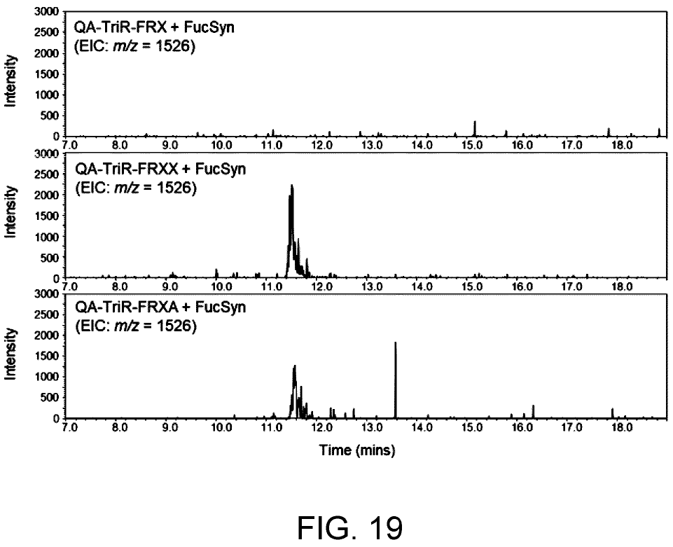

Next, using the QA-TriR scaffold, the C-28 tetrasaccharide chain was synthesised step-by step to verify the importance of the QsFucSyn enzyme for compound production. In each case, comparison of the product abundance showed that the QsFucSyn enzyme was important for boosting the content of the C-28 glycosylated products (FIG. 18). Using the QsFucSyn enzyme, the production of the full C-28 glycosides featuring either the terminal xylose or apiose were confirmed (FIG. 19).

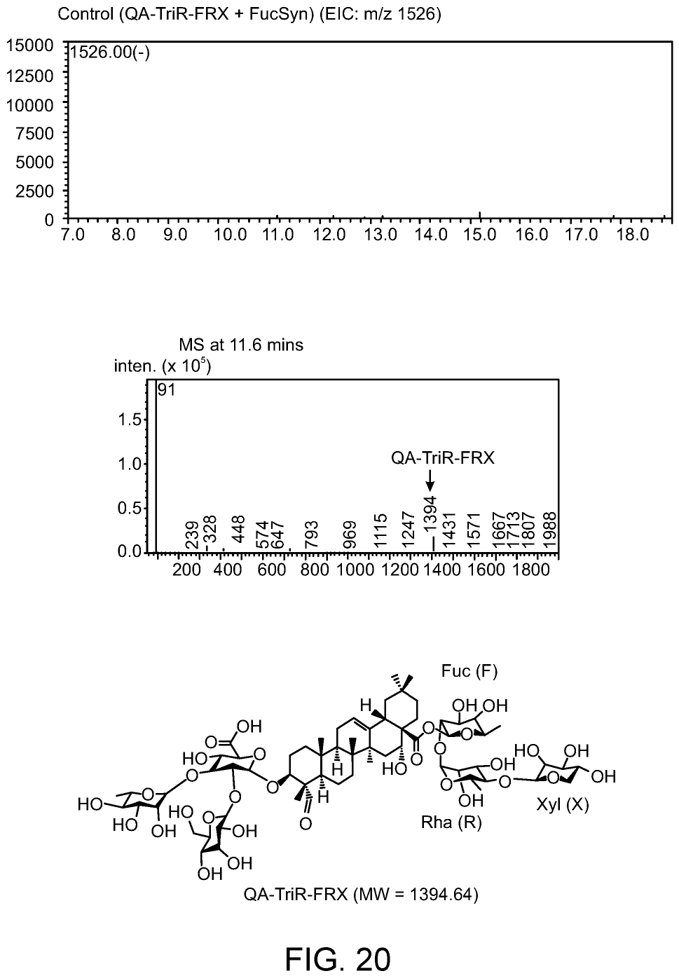

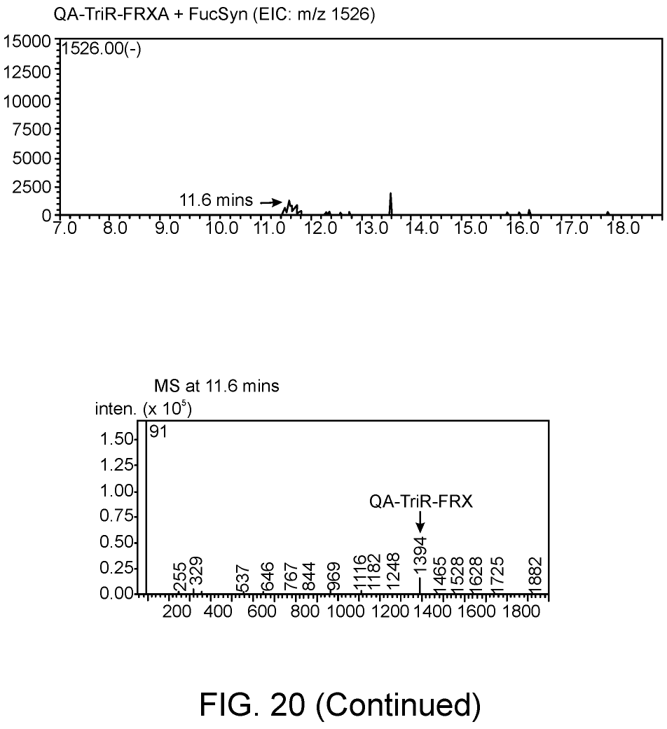

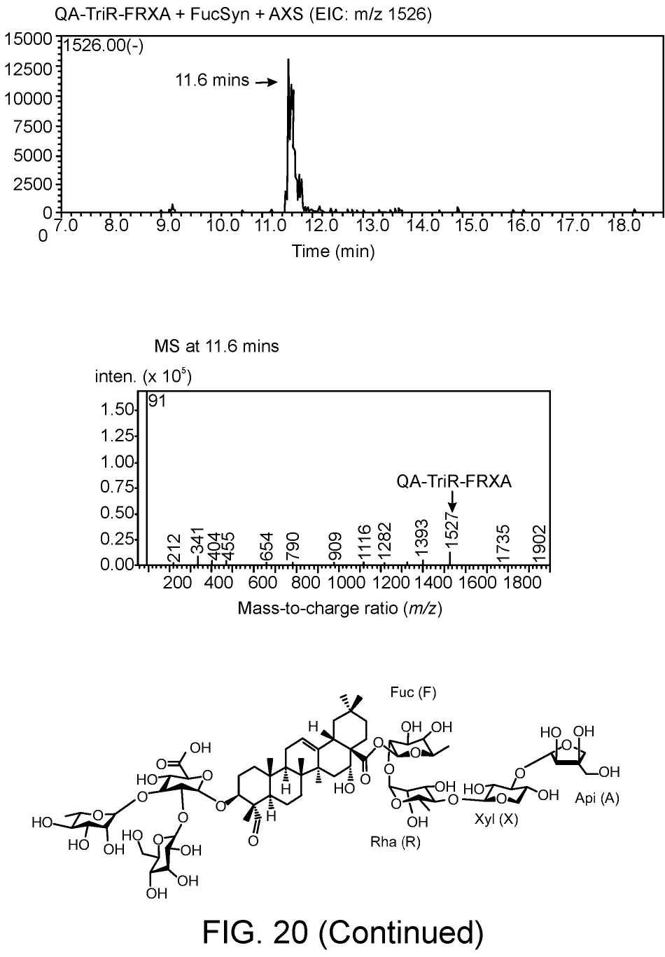

Finally, the importance of the QsAXS1 enzyme for boosting yields of the apiosylated product were again ascertained. Transient expression of the enzymes for production of QA-TriR-FRXA was performed in the presence or absence of the QsAXS1 enzyme. EIC analysis confirmed that only a small amount of QA-TriR-FRXA product (MW=1526.68) could be detected in the absence of QsAXS1, with the majority of the coeluting precursor QA-TriR-FRX (MW=1394.64) remaining in the sample at 11.6 mins (FIG. 20). By contrast, inclusion of QsAXS1 resulted in several-fold increase in the QA-TriR-FRXA product to become the major product at 11.6 minutes.

Following the identification of the five sugar transferases and the key QsAXS1/QsFucSyn enzymes necessary for enhancing production of the C-28 glycosylated products, we performed a large-scale vacuum infiltration as described previously (Reed et al., 2017, Stephenson et al., 2018) for each step in the production of the C-28 tetrasaccharide chain using the QA-TriR scaffold, in order to purify sufficient amounts of each target compound (QA-TriR-F, QA-TriR-FR, QA-TriR-FRX, QA-TriR-FRXX and QA-TriR-FRXA) for NMR analysis.

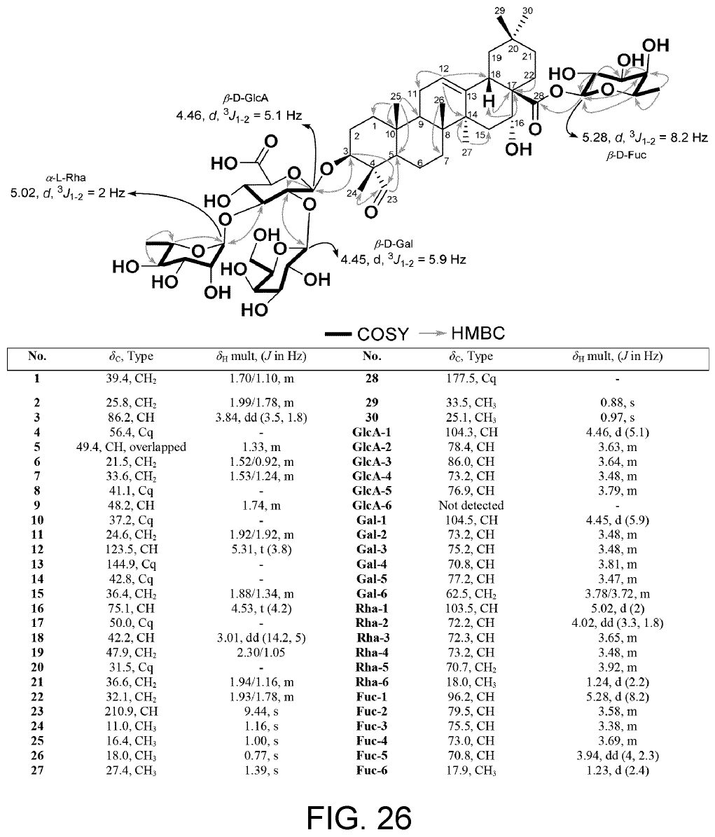

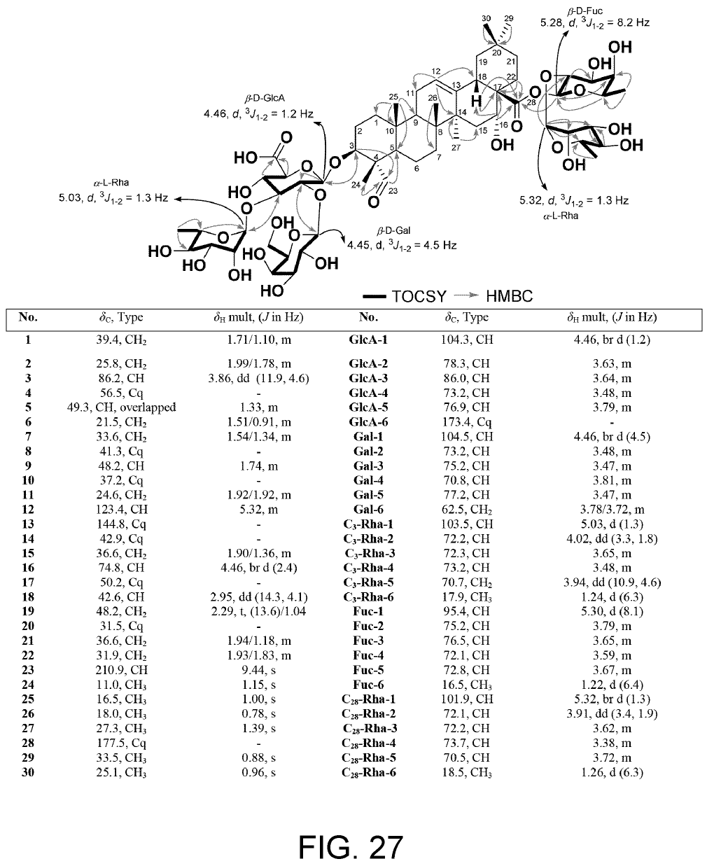

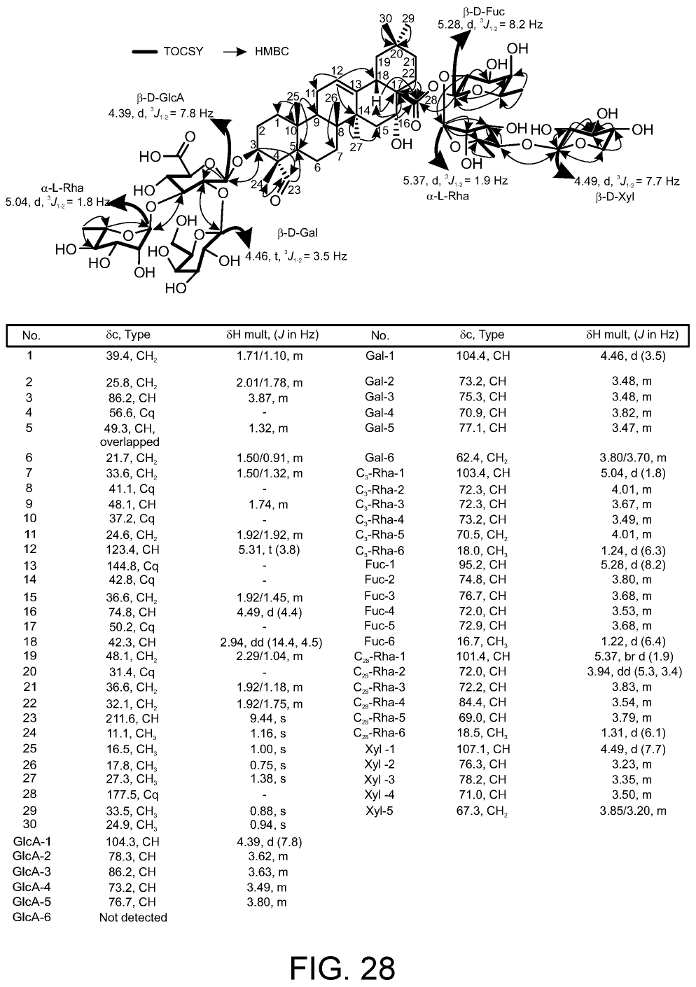

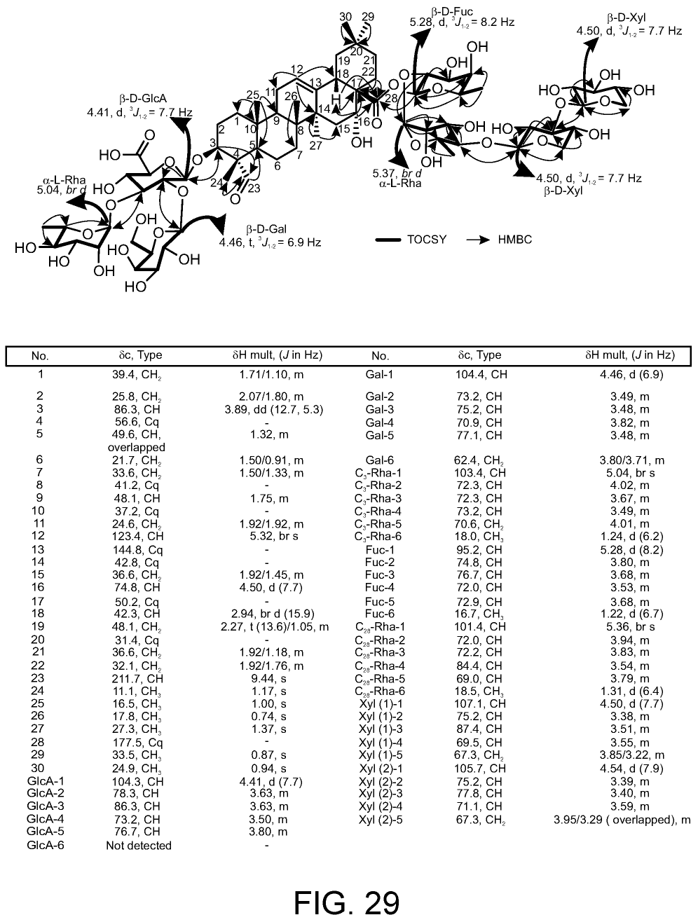

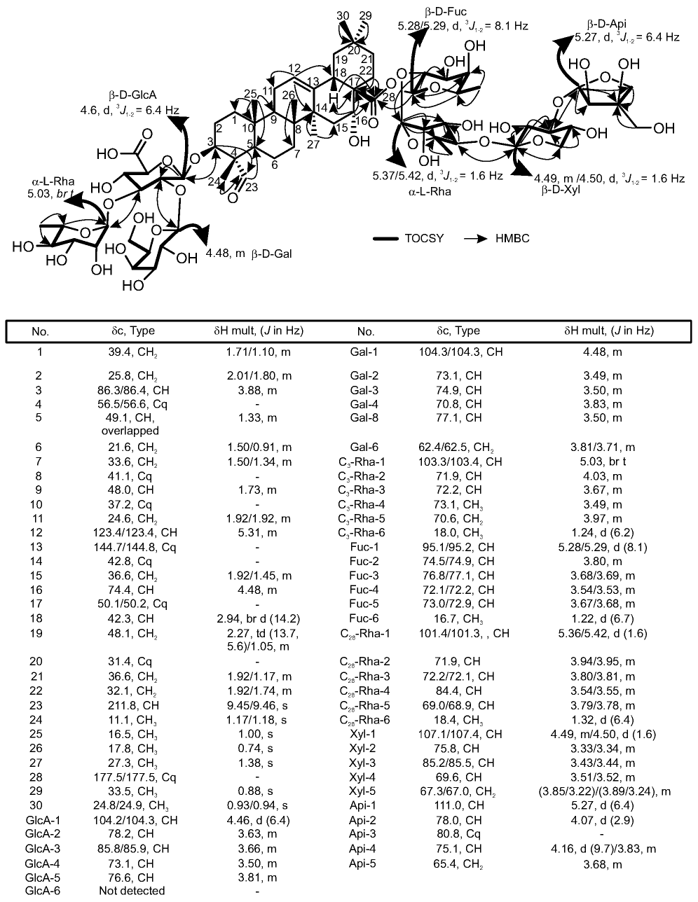

NMR analysis confirmed that the structure of QA-TriR-F is quillaic acid 3-O-{α-L-rhamnopyranosyl-(1->3)-[β-D-galactopyranosyl-(1->2)]-β-D-glucopyranosiduronic acid}-28-0-[β-D-fucopyranosyl] (FIG. 26); QA-TriR-FR is quillaic acid 3-O-{α-L-rhamnopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl} (FIG. 27); QA-TriR-FRX is quillaic acid 3-O-{α-L-rhamnopyranosyl-(1->3)-[3-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl}(FIG. 28); QA-TriR-FRXX is quillaic acid 3-O-{α-L-rhamnopyranosyl-(1->3)-[3-D-galactopyranosyl-(1->2)]-β-D-glucopyranosiduronic acid}-28-O-{β-D-xylopyranosyl-(1->3)-3-D-xylopyranosyl-(1->4)-α-L-rhamnopyranosyl-(1->2)-3-D-fucopyranosyl} (FIG. 29); and QA-TriR-FRXA is quillaic acid 3-O-{α-L-rhamnopyranosyl-(1->3)-[3-D-galactopyranosyl-(1->2)]-β-D-glucopyranosiduronic acid}-28-O-{β-D-apiofuranosyl-(1->3)-3-D-xylopyranosyl-(1->4)-α-L-rhamnopyranosyl-(1->2)-3-D-fucopyranosyl} (FIG. 30).

Part D: Further Enhancing the Activity of the QsFucSyn by Coexpression of ATCV-1

The QsFucSyn enzyme is related to several characterised SDR enzymes from other species, including the salutaridine reductase from poppy (56% amino acid identity), neomenthol dehydrogenases from Capsicum annuum (57% identity) and Mentha pipertia (55% identity) and two aldehyde reductases from Arabidopsis thaliana (both 61% identity). The substrates of these enzymes are varied, however it can be seen that in each case the enzymes catalyse the reduction of carbonyl groups to alcohols. The second step in the proposed biosynthesis of UDP-D-fucose from UDP-D-glucose involves a keto-reduction at the C-4 position (FIG. 15). It is possible that the QsFucSyn enzyme is performing a stereoselective reduction at C-4 of the UDP-4-keto-6-deoxy-D-glucose (a product that occurs naturally as an intermediate in UDP-L-rhamnose biosynthesis) once it has been added to the QA backbone. Alternatively, the QsFucSyn enzyme may be performing a stereoselective reduction at C-4 of the UDP-4-keto-6-deoxy-D-glucose to form UDP-D-fucose. With this reasoning in mind, increasing the availability of UDP-4-keto-6-deoxy-D-glucose in N. benthamiana could be expected to further enhance the activity of the QsFucSyn enzyme. The previously described ATCV-1 enzyme is a UDP-D-glucose 4,6-dehydratase (UGD) and produces UDP-4-keto-6-deoxy-D-glucose from UDP-d-glucose (Parakkottil Chothi et al., 2010).

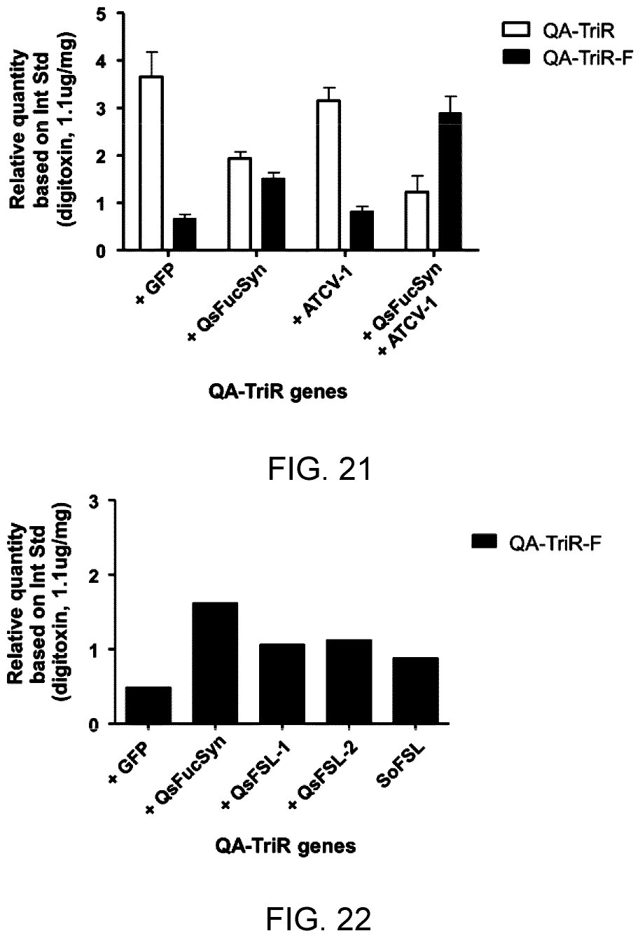

Therefore, to test whether ATCV-1 could enhance the activity of QsFucSyn, these two enzymes were transiently co-expressed with the enzyme set necessary for production of the QA-TriR-F product. The levels of QA-TriR-F were measured in leaf extracts and used to determine the effectiveness of this strategy. As anticipated, the combination of both ATCV-1 and QsFucSyn enhanced the levels of the QA-TriR-F product over expression of either strategy alone (FIG. 21). The increase in the QA-TriR-F product was concomitant with a drop in the levels of QA-TriR, suggesting that the increase was a direct result of increased fucosylation. This demonstrates that co-expression of a UDP-D-glucose 4,6-dehydratase with QsFucSyn is an effective strategy for enhancing the production of fucosylated saponins. This could be achieved with an enzyme similar to ATCV-1 (possessing standalone 4,6-dehydratase activity). Alternatively, such an enzyme could be generated by truncation of a plant UDP-L-rhamnose synthase. These enzymes normally convert UDP-D-glucose to UDP-L-rhamnose in a 3-step reaction carried out by a single large enzyme possessing 4,6-dehydratase, 3,5-epimerase and 4-reductase activity. However, the 4,6-dehydratase activity is encoded by the N-terminus of the RHM protein and can be decoupled from the latter two steps. An example of this is seen by use of a truncated variant of the Arabidopsis thaliana RHM2 gene (AT1G53500, normally 667 amino acids long). Removal of 297 amino acids from the C-terminus to leave the N-terminal 370 amino acids results in a functional protein possessing only UDP-d-glucose 4,6-dehydratase activity). This truncated variant is 60% identical to ATCV-1. Use of a truncated RHM gene may therefore be a viable alternative to ATCV-1.

Part E: Identification of FucSyn Homologues

To investigate the specificity of QsFucSyn, other homologues were investigated. Firstly, analysis of the Q. saponaria genome revealed fifteen homologues ranging from 52-91% identity at the amino acid level. Transcriptomic analysis revealed that most of these had very low FPKM expression values, suggesting that the enzymes might be pseudogenes. However, several did appear to be expressed to various degrees in different tissues. Consequently, two such candidates were cloned to investigate their FucSyn-like activity. These are named QsFucSyn-Like (QsFSL). QsFucSyn-Like means the candidates have 52-91% identity at the amino acid level to QsFucSyn. The first (QsFSL-1) is 82% identical to FucSyn at the amino acid level and the second (QsFSL-2) was 54% identical. Next, we investigated a QsFucSyn-Like protein in Saponaria officinalis, known colloquially as soapwort and member of the unrelated Caryophyllaceae family. S. officinalis is known to produce D-fucosylated saponins, therefore a homologue of QsFucSyn was identified in this plant (named SoFSL-1). All genes were amplified by PCR from cDNA from their respective plants, cloned into pEAQ-HT-DEST1 and transformed into A. tumefaciens for transient expression in N. benthamiana. The gene set for production of the QA-TriR-F product were transiently co-expressed. In addition, the various FSLs were also co-expressed and the impact on QA-TriR-F production was measured using LC-CAD (FIG. 22).

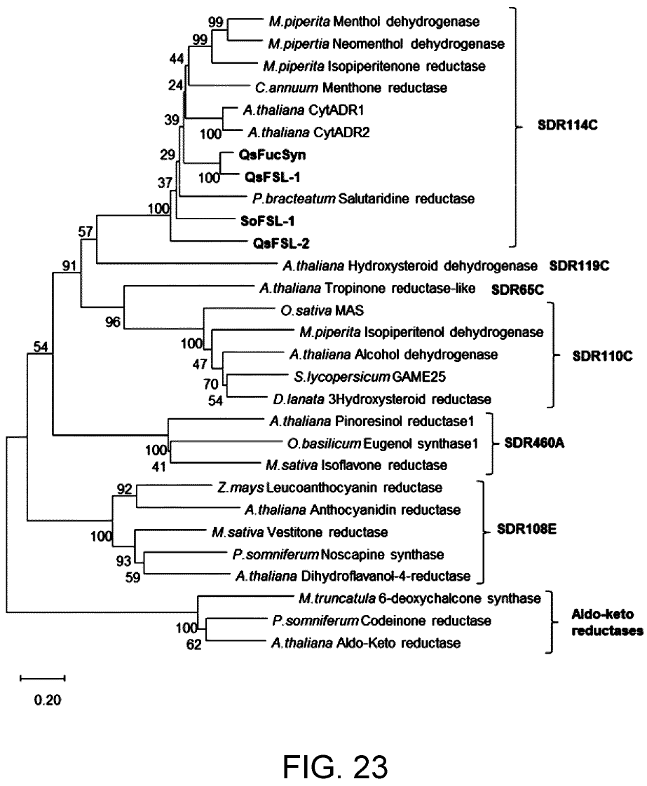

The analysis revealed that all of the tested FSL genes resulted in at least a two-fold increase in the fucosylated product relative to the negative control, although the original QsFucSyn resulted in the strongest increase. This provides strong evidence that proteins with homology to QsFucSyn may also be useful tools for enhancing fucosylation. Phylogenetic analysis of the QsFucSyn, QsFSL-1, QsFSL-2 and SoFSL-1 showed that these proteins are likely to form part of the SDR114C family (FIG. 23).

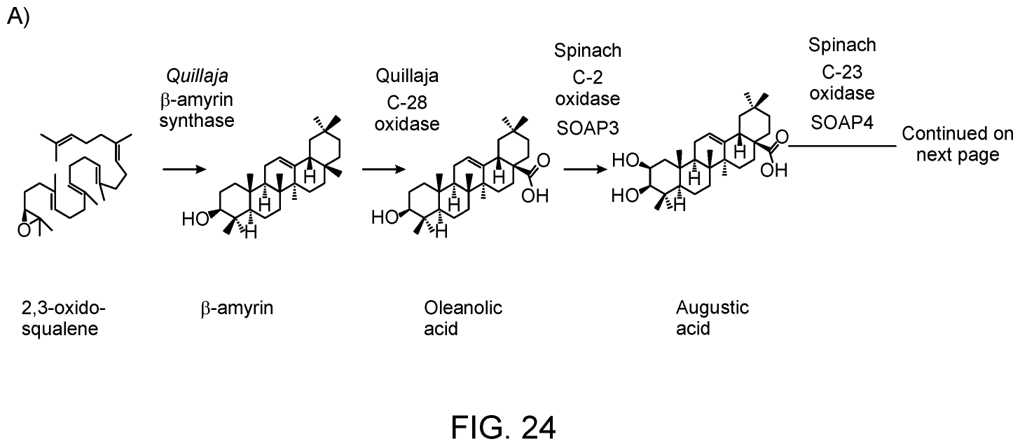

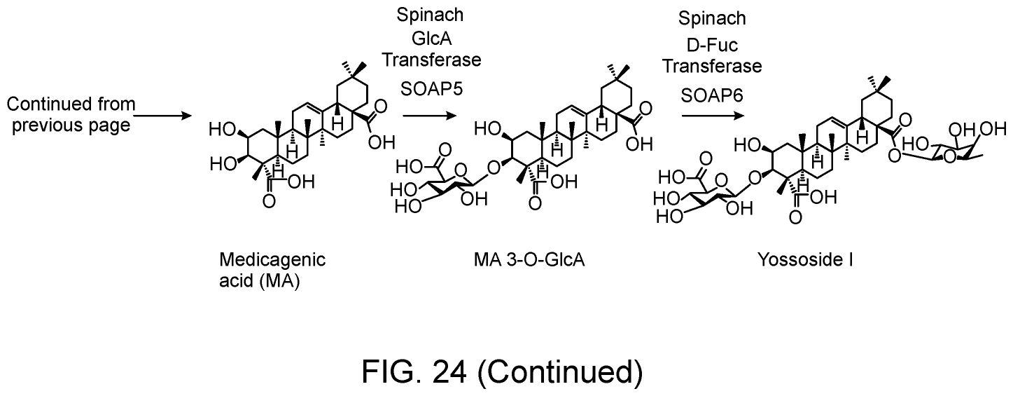

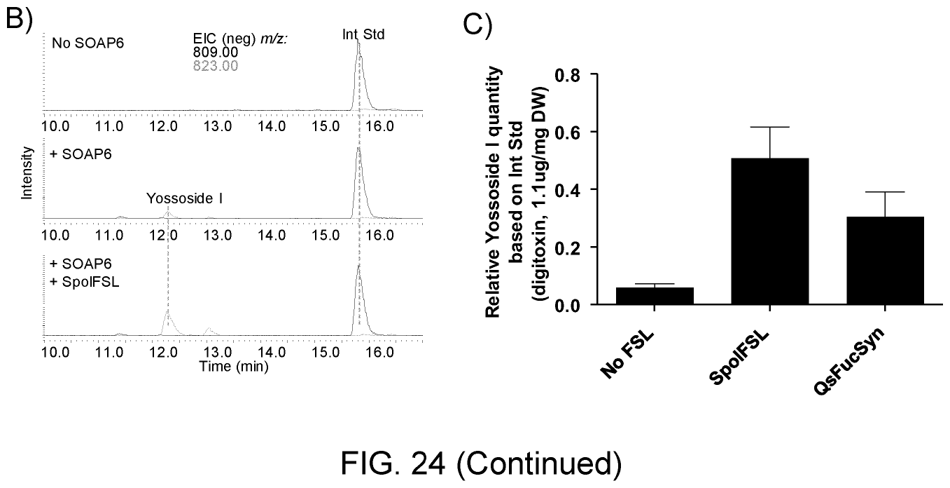

A QsFucSyn-Like protein in Spinacia oleracea was then investigated. SOAP6 is a D-fucosyltransferase and is involved in saponin (yossoside) biosynthesis in spinach (Spinacia oleracea). SOAP6 catalyses the C-28 D-fucosylation of Medicagenic acid-3-O-GlcA to form the product “Yossoside I” (Jozwiak, 2020) (FIG. 24). It has been noted that the function of SOAP6 may be impaired when transiently expressed in N. benthamiana, resulting in limited accumulation of Yossoside I. This may be due to limited availability of necessary sugar nucleotide precursors (i.e. UDP D-fucose).

The Yossoside genes show that strong co-expression and discovery of the known Yossoside pathway enzymes was enabled by performing a co-expression analysis using the early pathway genes (SOAP1, SOAP2 and CYP716A268v2) as bait (Jozwiak, 2020). The output of this co-expression analysis contains more than 1000 genes from spinach (Jozwiak, 2020). Although the original study did not identify any FucSyn-like enzyme involved in D-fucose biosynthesis, the co-expression data was analysed for presence of an SDR related to QsFucSyn. A single example was found in this dataset, with co-expression values above 0.9 to SOAP1 and CYP716A268v2. This enzyme is named herein Spinacia oleracea FucSyn-like (SpolFSL).

The SpoIFSL was cloned by PCR from spinach along with several other genes from the yossoside pathway necessary for production of Yossoside I. The early steps of Yossoside biosynthesis involve a β-amyrin synthase (SOAP1) and C-28 oxidase (SOAP2/CYP716A268) (FIG. 24). As these steps are shared with Quillaja saponin biosynthesis, only the yossoside-specific enzymes were cloned, including the C-2P oxidase (SOAP3/CYP72A655), C-23 oxidase (SOAP4/CYP72A654), C-3 glucuronosyltransferase (SOAP5) and C-28 D-fucosyltransferase (SOAP6). These yossoside genes were transiently co-expressed in N. benthamiana with the Quillaja β-amyrin synthase (QsbAS) and C-28 oxidase (QsCYP716-C-28). Subsequent analysis by LC-MS confirmed that Yossoside I could be detected in N. benthamiana and its presence was dependent on the presence of SOAP6 (FIG. 24). Furthermore, inclusion of SpoIFSL resulted in a substantial increase in the Yossoside I product (FIG. 24), indicating that SpoIFSL may participate in D-fucosylation, similar to the QsFucSyn enzyme. Similarly, inclusion of the QsFucSyn also resulted in an increase to Yossoside 1.

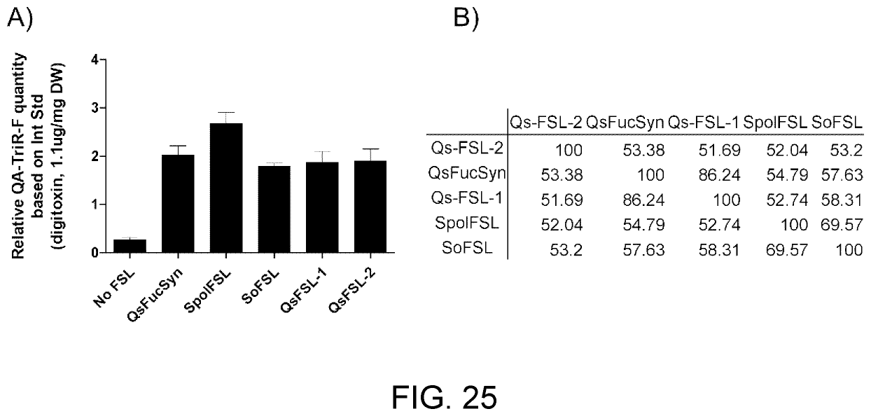

Following demonstration that the SpolFSL enzyme was capable of boosting the Yossoside I product, the ability of SpolFSL to boost a non-spinach D-fucosylated product was investigated. The enzymes needed to produce QA-TriR-F were transiently expressed in N. benthamiana. Co-expression of the SpolFSL enzyme was found to substantially increase the amount of QA-TriR-F compared to the QA-TriR-F enzymes-only (i.e. No FSL) control. The boosted levels of QA-TriR-F were comparable to the boosting achieved with a number of other FucSyn-like enzymes from different species, including the Quillaja saponaria FucSyn (QsFucSyn), FucSyn-like 1 (QsFSL-1) and FucSyn-like 2 (QsFSL-2) enzymes and the Saponaria officinalis FucSyn-like (SoFSL) (FIG. 25). Pairwise identities (protein) are shown in FIG. 25. Together these results demonstrate the ability of FucSyn-like proteins from across the plant kingdom to boost the levels of D-fucosylated products.

Materials and Methods

Primers and Cloning

The genes encoding the enzymes described herein (Qs-28-O-FucT, Qs-28-O-RhaT, Qs-28-O-XylT3, Qs-28-O-XylT4, Qs-28-O-ApiT4, QsFucSyn, QsFSL-1, QsFSL-2, SoFSL-1 and QsAXS1) were amplified by PCR from cDNA derived from leaf tissue of Q. saponaria.

PCR was performed using the primers detailed in Table 1 and iProof polymerase with thermal cycling according to the manufacturer's recommendations. The resultant PCR products were purified (Qiagen PCR cleanup kit) and each cloned into the pDONR207 vector using BP clonase according to the manufacturer's instructions. The BP reaction was transformed into E. coli and the resulting transformants were cultured and the plasmids isolated by miniprep (Qiagen). The isolated plasmids were sequenced (Eurofins) to verify the presence of the correct genes. Next each of the three genes were further subcloned into the pEAQ-HT-DEST1 expression vector using LR clonase. The resulting vectors were used to transform A. tumefaciens LBA4404 by flash freezing in liquid N₂.

TABLE 1
Primers used to clone the sequences.

| Name | Sequence |
|---|---|
| Qs-28-O- | 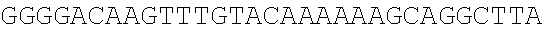 |
| FucT_attB1F | ATGGAGAATGGGAGAGTTTACAAATCC |
| Qs-28-O- | 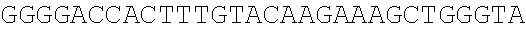 |
| FucT_attB2R | TCAAGTTGTGATTCCAGCAATGAATTC |
| Qs-28-O- | 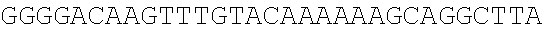 |
| RhaT_attB1F | ATGGCAAAAACTGATAAGCAGCTTC |
| Qs-28-O- | 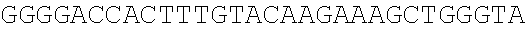 |
| RhaT_attB2R | TTAAATTTGGAAAGGTTCCCTTTTG |
| Qs-28-O- | 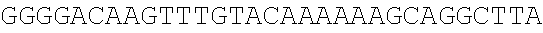 |
| XylT3_attB1F | ATGGCTGCTGCAGCTCC |
| Qs-28-O- | 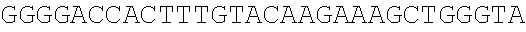 |
| XylT3_attB2R | TTAATTCCTCTTAAGAGACCTGTAATTTTTGAAG |
| Qs-28-O- | 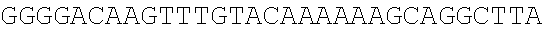 |
| XylT4_attB1F | ATGGACTCCACCCACTTGC |
| Qs-28-O- | 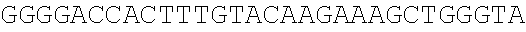 |
| XylT4_attB2R | TCAAATTGTTTTTGTTTTCAGCTT |
| Qs-28-O- | 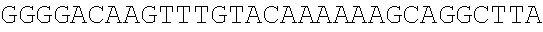 |
| ApiT4_attB1F | ATGGACTCCACCCACTTGCAGCC |
| Qs-28-O- | 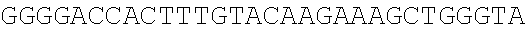 |
| ApiT4_attB2R | TCAAATTGTTTTTGTTTTCAGCTTCG |
| QsAXS1_attB1F | 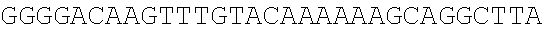 |
| | ATGGCGTCGGCGTCA |
| QsAXS1_attB2R | 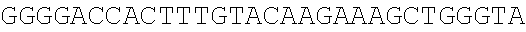 |
| | CTAGCTGGCAACTGGTTTCG |
| QsFucSyn_attB1F | 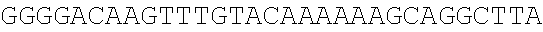 |
| | ATGGCAGAAGCAACGCAGAGGTATG |
| QsFucSyn_attB2R | 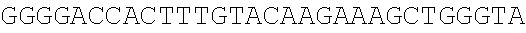 |
| | TCAAAATGGTGCTTCTTCTGTCCTG |
| QsFSL-1_attB1F | 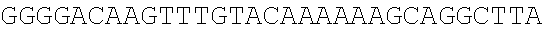 |
| | ATGGCAGAAGCAACAGAGAGG |
| QsFSL-1_attB2R | 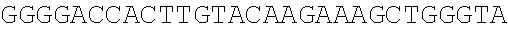 |
| | TCAAAATGGTGTCTCTTCAGTCCTG |
| QsFSL-2_attB1F | 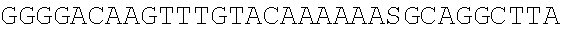 |
| | ATGGGTTCAGATGGAAGGGATG |
| QsFSL-2_attB2R | 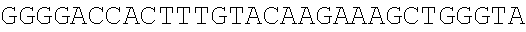 |
| | TTAAAATTCTGCCTGTTGAGTACTATC |
| SoFSL-1_attB1F | 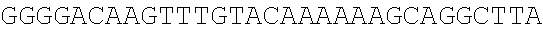 |
| | ATGGCTGAAGCATCCTCATTTC |
| SoFSL-1_attB2R | 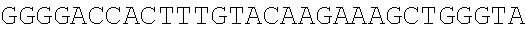 |
| | TCATTCAAATGGAGTTACTTCGTTTCG |

Gene specific sequences are shown in black, while the attB sites required for Gateway ® cloning are shown in grey.

Agroinfiltration of N. benthamiana Leaves

Agroinfiltration was performed using a needleless syringe as previously described (Reed et al., 2017). All genes were expressed from pEAQ-HT-DEST1 binary expression vectors (Sainsbury et al., 2009) in A. tumefaciens LBA4404 as described above. Cultivation of bacteria and plants is as described in (Reed et al., 2017).

### Preparation of N. benthamiana Leaf Extracts for LC-MS Analysis

Leaves were harvested 5 days after agroinfiltration and lyophilised. Dried leaf material (10 mg per sample) was disrupted with tungsten beads at 1000 rpm for 1 min (Geno/Grinder 2010, Spex SamplePrep). Metabolites were extracted in 550 μL 80% methanol containing 20 μg/mL of internal standard (digitoxin (Sigma-Aldrich)) and incubated for 20 min at 18° C., with shaking at 1400 rpm (Thermomixer Comfort, Eppendorf). Each sample was defatted by partitioning twice with 400 μL hexane. The upper phase was discarded and the lower aqueous phase was dried under vacuum at 40° C. for 1 hour (EZ-2 Series Evaporator, Genevac). Dried material was resuspended in 75 μL of 100% methanol and filtered at 12, 500×g for 30 sec (0.2 μm, Spin-X, Costar). The filtrate (50 μL) was combined with 50 μL 50% methanol in glass vials and analysed as detailed below.

HPLC-CAD-MS analysis of N. benthamiana leaf extracts Analysis was carried out using a Shimadzu Prominence HPLC system with single quadrupole mass spectrometer LCMS-2020 (Shimadzu) and Corona Veo RS Charged Aerosol Detector (CAD) (Dionex). Detection: MS (dual ESI/APCI ionization, desolvation line temperature=250° C., nebulizing gas flow=15 L·min⁻¹, heat block temperature=400° C., spray voltage Positive 4.5 kV, Negative−3.5 kV) CAD data collection rate 10 Hz, filter constant 3.6 s, 925 evaporator temp. 35° C., ion trap voltage 20.5 V. Method: Solvent A: [H2O+0.1% formic acid] Solvent B: [acetonitrile (CH3CN)+0.1% formic acid. Injection volume: 10 μL. Gradient: 15% [B] from 0 to 1.5 min, 15% to 60% [B] from 1.5 to 26 min, 60% to 100% [B] from 26 to 26.5 min, 100% [B] from 26.5 to 28.5 min, 100% to 15% [B] from 28.5 to 29 min, 35% [B] from 29 to 30 min. Method was performed using a flow rate of 0.3 mL·min−1 and a Kinetex column 2.6 μm XB-C18 100 Å, 50×2.1 mm (Phenomenex). Analysis was performed using LabSolutions software (Shimadzu). Where quantification of the QA-TriR and QA-TriR-F products was performed, the peak areas of the products were measured in the CAD traces and divided by the peak area of the internal standard (digitoxin).

Large Scale Vacuum Infiltration of N. benthamiana

Plants were infiltrated by vacuum as previously described (Reed et al., 2017, Stephenson et al., 2018) with A. tumefaciens LBA4404 strains carrying pEAQ-HT-DEST1 expression vectors harbouring relevant genes as detailed in Table 2. Plants were harvested after 5 days and leaves were lyophilised.

Purification of Compounds from Large Scale Infiltrations of N. benthamiana

Organic solvents used for extraction and flash chromatography were reagent grade and used directly without further distillation. Dried leaf material from large scale infiltrations were initially extracted by hexane for defatting, followed by subsequent exhaustive extraction using methanol/water (90/10 for QA-TriR-F and QA-TriR-FR, and 80/20 for QA-TriR-FRX, QA-TriR-FRXX and QA-TriR-FRXA) under refluxing at 95° C. for 2 days. The crude methanolic extract was combined and evaporated under reduced pressure and re-dissolved in a minimum of methanol and diluted with the equivalent volume of water, then partitioned using separation funnel against hexane, dichloromethane, ethyl acetate and n-butanol. The butanol layer was recollected and evaporated under reduced pressure and re-dissolved in the least amount of methanol and saturated with cold acetone to precipitate an enriched saponins crude fraction. This fraction was subjected to reparative chromatographic purifications by reversed phase using Phenomenex Luna C₁₈ columns (250×21.2 and 250×10 mm i.d.; 5 μm; for preparative and semi-preparative chromatography respectively) with an eluent system of water/acetonitrile containing 0.1% formic acid with the following compound-specific conditions: for QA-TriR-F, this fraction was separated on an Agilent semipreparative C₁₈-HPLC [(gradient, 90/10→30/70, over 35 min, 3 mL/min), (isocratic, 60:40, 1 mL/min)]; for QA-TriR-FR and QA-TriR-FRX, the fraction was separated as for QA-TriR-F except the gradient was 90/10→30/70 over 50 min, 3 mL/min; for QA-TriR-FRXX, this fraction was separated on a Agilent preparative C₁₈-HPLC with a gradient of 90/10→30/70, over 17 min, 25 mL/min; and for QA-TriR-FRXA, this fraction was separated on a preparative and semi-preparative C₁₈-HPLC with a gradient of 90/10→30/70, over 17 min, 25 mL/min, and by isocratic 60/40, over 30 min, 2 mL/min. Dried leaf weight and the purified amount of each isolated compound are detailed in Table 2.

NMR Analysis

10D and 20 NMR spectra were recorded on Bruker Avance 600 MHz spectrometer equipped with a BBFO Plus Smart probe and a triple resonance TCI cryoprobe, respectively (JIC, UK). The chemical shifts are relative to the residual signal solvent (MeOH-d4: δH 3.31; δC 49.15).

TABLE 2
Strains used for large-scale vacuum infiltrations, number of plants infiltrated,
dry leaf weight and amount of product purified for each compound.

| | | Number of | | |
|---|---|---|---|---|
| | Combination of Agrobacterium tumefaciens | vacuum- | | |
| | strains carrying the pEAQ-HT-DEST1 constructs | infiltrated | Dry leaf | Amount |
| Compound | for the following genes: | plants | weight | isolated |
| QA-TriR-F | AstHMGR (SEQ ID No 15), QsbAS (SEQ ID NO | 124 | 40 g | 1 mg |
| | 17), QsCYP716-C-28 (SEQ ID NO 19), | | | |
| | QsCYP716-C-16a (SEQ ID NO 21), QsCYP714- | | | |
| | C-23 (SEQ ID NO 23), CSIG2 (SEQ ID NO 27), | | | |
| | Qs-3-O-GalT (SEQ ID NO 29), Qs_0283850 | | | |
| | (SEQ ID NO 33), QsFucSyn (SEQ ID NO 11) | | | |
| | and Qs-28-O-FucT (SEQ ID NO 1) | | | |
| QA-TriR-FR | AstHMGR (SEQ ID No 15), QsbAS (SEQ ID NO | 112 | 46 g | 2.2 mg |
| | 17), QsCYP716-C-28 (SEQ ID NO 19), | | | |
| | QsCYP716-C-16α (SEQ ID NO 21), QsCYP714- | | | |
| | C-23 (SEQ ID NO 23), CSIG2 (SEQ ID NO 27), | | | |
| | Qs-3-O-GalT (SEQ ID NO 29), Qs_0283850 | | | |
| | (SEQ ID NO 33), QsFucSyn (SEQ ID NO 11), | | | |
| | Qs-28-O-FucT (SEQ ID NO 1) and Qs-28-O- | | | |
| | RhaT (Seq ID NO 3) | | | |
| QA-TriR- | AstHMGR (SEQ ID No 15), QsbAS (SEQ ID NO | 202 | 65 g | 1.6 mg |
| FRX | 17), QsCYP716-C-28 (SEQ ID NO 19), | | | |
| | QsCYP716-C-16a (SEQ ID NO 21), QsCYP714- | | | |
| | C-23 (SEQ ID NO 23), CsIG2 (SEQ ID NO 27), | | | |
| | Qs-3-O-GalT (SEQ ID NO 29), Qs_0283850 | | | |
| | (SEQ ID NO 33), QsFucSyn (SEQ ID NO 11), | | | |
| | Qs-28-O-FucT (SEQ ID NO 1), Qs-28-O-RhaT | | | |
| | (Seq ID NO 3) and Qs-28-O-XyIT3 (Seq ID NO | | | |
| | 5) | | | |
| QA-TriR- | AstHMGR (SEQ ID No 15), QsbAS (SEQ ID NO | 105 | 58 g | 13.2 mg |
| FRXX | 17), QsCYP716-C-28 (SEQ ID NO 19), | | | |
| | QsCYP716-C-16a (SEQ ID NO 21), QsCYP714- | | | |
| | C-23 (SEQ ID NO 23), CSIG2 (SEQ ID NO 27), | | | |
| | Qs-3-O-GalT (SEQ ID NO 29), Qs_0283850 | | | |
| | (SEQ ID NO 33), QsFucSyn (SEQ ID NO 11), | | | |
| | Qs-28-O-FucT (SEQ ID NO 1), Qs-28-O-RhaT | | | |
| | (Seq ID NO 3), Qs-28-O-XyIT3 (Seq ID NO 5) | | | |
| | and Qs-28-O-XyIT4 (Seq ID NO 7) | | | |
| QA-TriR- | AstHMGR (SEQ ID No 15), QsbAS (SEQ ID NO | 105 | 58 g | 13.2 mg |
| FRXA | 17), QsCYP716-C-28 (SEQ ID NO 19), | | | |
| | QsCYP716-C-16a (SEQ ID NO 21), QsCYP714- | | | |
| | C-23 (SEQ ID NO 23), CsIG2 (SEQ ID NO 27), | | | |
| | Qs-3-O-GalT (SEQ ID NO 29), Qs_0283850 | | | |
| | (SEQ ID NO 33), QsFucSyn (SEQ ID NO 11), | | | |
| | Qs-28-O-FucT (SEQ ID NO 1), Qs-28-O-RhaT | | | |
| | (Seq ID NO 3), Qs-28-O-XyIT3 (Seq ID NO 5), | | | |
| | Qs-28-O-ApiT4 (Seq ID NO 9) and QsAXS1 | | | |
| | (Seq ID NO 13) | | | |

### Synthesis of the UDP-α-D-Fucose Standard

Preparation of UDP-α-D-fucose standard was performed using a 1-pot enzymatic procedure as previously described (Errey et al., 2004). Briefly, pyruvate kinase (50 U), inorganic phosphatase (5 U), galactose-1-phosphate uridylyltransferase (75 U), glucose-1-phosphate uridylyltransferase (5 U) and galactose kinase (100 U) were combined in a buffer (50 mM HEPES, pH 8.0, 5 mM KCl, 10 mM MgCl₂) containing UTP (2 mg/mL), ATP (0.1 mg/mL), PEP (1.4 mg/mL), UDP-α-D-glucose (0.1 mg/mL) and D-fucose (1 mg/mL). The reaction (total volume 1 mL) was left at room temperature overnight. The following day, purification of UDP-α-D-fucose was performed by HPLC as detailed below. The sample was diluted 1:1 with methanol and applied on a Poros HQ 50 column (50×10 mm, column volume (CV)=3.9 mL). The column was equilibrated with 5 CV of 5 mM NH₄HCO₃ buffer at a flow rate of 8 ml/min. Following the injection of the sample, a linear gradient was run (8 mL/min) as follows: Solvent A [5 mM NH₄HCO₃], Solvent B [250 mM NH₄HCO₃]. Gradient: 0% [B] to 100% [B] over 15 CV and held for 5 CV. The column was equilibrated in 100% [B] for an additional 3 CV between each run. Detection of UDP-α-D-fucose was performed by monitoring absorption at 265 nm.

The identity of UDP-α-D-fucose was confirmed by high resolution mass spectrometry and ¹H NMR and found to be in accordance with the literature (Errey et al., 2004).

Sugar Nucleotide Extraction from N. benthamiana Leaves

Leaves of N. benthamiana plants (approximately 6 weeks old) were infiltrated with a solution of either 50 mM D-fucose (Glycon Biochemicals), or water. After 2 days, infiltrated leaves were harvested, and 2 g of leaf material was flash frozen in liquid N₂. Leaves were spiked with 2 μg of an internal standard (UDP-2-acetamido-2-deoxy-α-D-glucuronic acid (UDP-GlcNAcA)) and ground to a fine powder using a pestle and mortar. Sodium fluoride solution (10 mL 40 mM) was added and samples were incubated on ice for 1 hr with intermittent shaking/vortexing and three cycles of sonication (60 sec each, 4° C.). Samples were centrifuged at 29,000×g for 20 min at 4° C. and the supernatant was transferred to a glass round bottom flask, frozen and lyophilised overnight. The following day, samples were dissolved in 9% aqueous butan-1-ol (6 mL) and extracted with 90% butan-1-ol (2 mL). Samples were centrifuged at 2000×g for 10 min at 4° C. to aid separation of the layers, with the upper organic layer discarded each time. To completely remove lipophilic compounds the extraction was repeated 3 times. The lower aqueous layers were combined and transferred to a pear-shaped flask, frozen and lyophilised overnight. The dried samples were dissolved in 500 μL ammonium bicarbonate (5 mM) and sugar nucleotides were extracted using solid phase extraction (SPE) (SupelClean ENVI-Carb SPE tubes, 250 mg) as previously described (Räbinä et al., 2001).

Briefly, columns were conditioned with a solution of 80% acetonitrile and 0.1% trifluoroacetic acid (3 mL) followed by water (2 mL). Samples were loaded onto the column to adsorb the sugar nucleotides and the column was washed with water (2 mL), followed by 25% acetonitrile (2 mL) and 50 mM triethylammonium acetate (TEAA) buffer pH 7.0 (2 mL). Finally, sugar nucleotides were eluted with a solution of 25% acetonitrile in 50 mM TEAA buffer, pH 7.0 (1.5 mL). Samples were filtered through a 0.45 μm PTFE disc filters, frozen and lyophilised. Samples were dissolved in a solution of 0.3% formic acid, pH 9.0 with NH₄OH (50 μL, 5 mM) prior to analyses by LC-MS as detailed below. Standards of sugar nucleotides were used at a concentration of 10 μM.

Sugar Nucleotide Profiling of N. benthamiana Leaf Extracts.

Analysis of sugar nucleotide was performed as detailed in (Rejzek et al., 2017). Briefly, ESI-MS/MS analysis was performed using a Waters Xevo TQ-S system in negative ion mode (capillary voltage of 1.5 kV, 500° C. desolvation temperature, 1000 L/h desolvation gas, 150 L/h cone gas, and 7bar nebulizer pressure). Chromatography was performed using a ThermoFisher Hypercarb™ column (1×100 mm, particle size 3 μm) with a flow rate of 80 μL/min and the following mobile phase: Solvent A [0.3% Formic acid, pH 9.0 with NH₄OH], Solvent B [Acetonitrile]. Gradient: 2% [B] to 15% [B] from 0 to 20 min, 15% [B] to 50% [B] from 20-26 min, 50% [B] to 90% [B] from 26-27 min and held at 90% [B] until 30 min. The column was re-equilibrated from 90% [B] to 2% [B] from 30-31 min and held at 2% [B] until 50 mins.

Primers and Cloning of Spinach Genes

Spinach seeds were purchased from a local garden centre (Norwich, UK) and sown on seedling compost and germinated at 22° C. Leaves were harvested at approximately two weeks old and RNA was extracted using a Plant RNeasy kit (Qiagen) and used for synthesis of cDNA. Cloning of the yossoside biosynthetic genes SOAP3-6 and the SpolFSL was performed using the primers as detailed in Table 3. Genes were cloned into the binary expression vector pEAQ-HT-DEST1 and transformed into A. tumefaciens LBA4404 as described in the “primers and cloning” and “agroinfiltration of N. benthamiana leaves”. Transient expression and LC-MS/CAD analysis was performed as detailed in the “HPLC-CAD-MS of N. benthamiana leaf extracts” section. Peaks were quantified by measuring the peak area of the compound of interest by CAD and dividing by the peak of the internal standard (digitoxin 1.1 mg/g per dry leaf weight). The adjusted peak areas from all replicates (n=3) were then averaged. The pairwise percentage sequence identities were calculated using Clustal Omega (v 1.2.4).

TABLE 3
Primers
Primers used for cloning spinach genes.

| Name | Sequence |
|---|---|
| SOAP3_attB1F: | 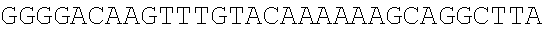 |
| | ATGATAGAAATCGGGTATATTGTAAAATG |
| SOAP3_attB2R: | 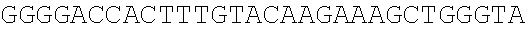 |
| | TTAGTCCCTGAGCTTATGTATAATG |
| SOAP4_attB1F: | 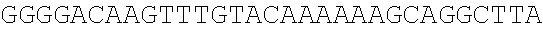 |
| | ATGATTTCAAAGAGCGCGAG |
| SOAP4_attB2R: | 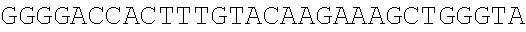 |
| | TTAAAATCGATGTAAAATAATGTGGGC |
| SOAP5_attB1F: | 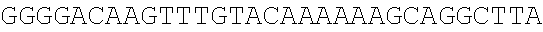 |
| | ATGGCAACTTCTCACATTCGC |
| SOAP5_attB2R: | 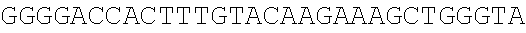 |
| | TTATAACCATCCCTTAACAACAGG |
| SOAP6_attB1F: | 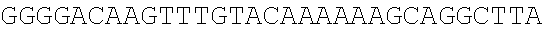 |
| | ATGACGGGAAAAGGAAGAACG |
| SOAP6_attB2R: | 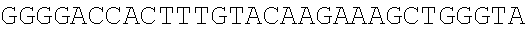 |
| | TTAGGAGGACGCAAGCCAGTTAATG |
| SpolFSL_attB1F: | 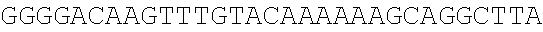 |
| | ATGGCTGAACAATCCAACTTTC |
| SpolFSL_attB2R: | 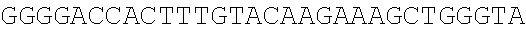 |
| | TTATTCATATGAAGAAACTTCGCTTC |

Gene specific sequences are shown in black, while the attB sites required for Gateway ® cloning are shown in grey.

### Abbreviations

AaFCD—Aggregatibacter actinomycetemcomitans NDP-4-keto-6-deoxyglucose 4-ketoreductase
Apif—D-Apiofuranose
Araf—L-Arabinofuranose
AstHMGR—Avena strigosa truncated 3-hydroxy-3-methyl-glutyryl-CoA reductase
ATCV-1—Acanthocystis turfacea chlorella virus 1 UDP-D-glucose 4,6-dehydratase
AtFCD—Anoxybacillus tepidamans NDP-4-keto-6-deoxyglucose 4-ketoreductase
DN20529_c0_g2_i8—Q. saponaria QA-Di α-1,3-L-rhamnosyltransferase
EcFCD—Escherichia coli NDP-4-keto-6-deoxyglucose 4-ketoreductase
FR—a disaccharide of a β-D-fucose and a α-L-rhamnose
FRX—a trisaccharide of a β-D-fucose, α-L-rhamnose and a β-D-xylose
FRXX—a tetrasaccharide of β-D-fucose, α-L-rhamnose, and two β-D-xylose
FRXA—a tetrasaccharide of β-D-fucose, α-L-rhamnose, β-D-xylose and a β-D-apiose
FRXX/A—a tetrasaccharide which is FRXX or FRXA.
Fucp—D-Fucopyranose
FucSyn—enzyme boosting the production of fucosylated saponins
FSL—FucSyn-Like
Galp—D-Galactopyranose
GlcpA—D-Glucopyranuronic acid
Glcp—D-Glucopyranose
Gyp—Gypsogenic acid
Gyp-TriX-G—3-O-{β-D-xylopyranosyl-(1->3)-[β-D-galactopyranosyl-(1->2)]-β-D-glucopyranosiduronic acid}-28-O-{β-D-glucopyranosyl ester}-gypsogenic acid
Gyp-TriX-GR—3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-glucopyranosyl ester}-gypsogenic acid
Gyp-TriX-GRX—3-O-{β-D-xylopyranosyl-(1->3)-[β-D-galactopyranosyl-(1->2)]-β-D-glucopyranosiduronic acid}-28-O-{β-D-xylopyranosyl-(1->4)-α-L-rhamnopyranosyl-(1->2)-β-D-glucopyranosyl ester}-gypsogenic acid
NSE—Sugar nucleotide interconverting enzyme
OS—2,3-oxidosqualene
OSC—oxidosqualene cyclase
QA—Quillaic acid
QA derivative
   QA-Di—3-O-{β-D-galactopyranosyl-(1->2)-β-D-glucopyranosiduronic acid}-quillaic acid
   QA-Di-F—3-O-{β-D-galactopyranosyl-(1->2)-β-D-glucopyranosiduronic acid}-28-O-{β-D-fucopyranosyl ester}-quillaic acid
   QA-Di-FR—3-O-{β-D-galactopyranosyl-(1->2)-β-D-glucopyranosiduronic acid}-28-O-{α-L-rhamnopyranosyl-(1->2)-β-D-fucopyranosyl ester}-quillaic acid
   QA-Di-FRX—3-O-{β-D-galactopyranosyl-(1->2)-β-D-glucopyranosiduronic acid}-28-O-{β-D-xylopyranosyl-(1->4)-α-L-rhamnopyranosyl-(1->2)-β-D-fucopyranosyl ester}-quillaic acid
   QA-Di-FRXA—3-O-{β-D-galactopyranosyl-(1->2)-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid
   QA-Di-FRXX—3-O-{β-D-galactopyranosyl-(1->2)-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid
   QA-Mono—3-O-{β-D-glucopyranosiduronic acid}-quillaic acid
   QA-Mono-F—3-O-{β-D-glucopyranosiduronic acid}-28-O-{β-D-fucopyranosyl ester}-quillaic acid
   QA-Mono-FR—3-O-{β-D-glucopyranosiduronic acid}-28-O-{α-L-rhamnopyranosyl-(1->2)-β-D-fucopyranosyl ester}-quillaic acid
   QA-Mono-FRX—3-O-{β-D-glucopyranosiduronic acid}-28-O-{β-D-xylopyranosyl-(1->4)-α-L-rhamnopyranosyl-(1->2)-β-D-fucopyranosyl ester}-quillaic acid
   QA-Mono-FRXA—3-O-{β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid
   QA-Mono-FRXX—3-O-{β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid
   QA-TriR—3-O-{α-L-rhamnopyranosyl-(1->3)-[3-D-galactopyranosyl-(1->2)]-β-D-glucopyranosiduronic acid}-quillaic acid
   QA-TriR-F—3-O-{α-L-rhamnopyranosyl-(1->3)-[3-D-galactopyranosyl-(1->2)]-3-D-glucopyranosiduronic acid}-28-O-{β-D-fucopyranosyl ester}-quillaic acid
   QA-TriR-FR—3-O-{α-L-rhamnopyranosyl-(1->3)-[3-D-galactopyranosyl-(1->2)]-3-D-glucopyranosiduronic acid}-28-O-{α-L-rhamnopyranosyl-(1->2)-β-D-fucopyranosyl ester}-quillaic acid
   QA-TriR-FRX—3-O-{α-L-rhamnopyranosyl-(1->3)-[3-D-galactopyranosyl-(1->2)]-β-D-glucopyranosiduronic acid}-28-O-{β-D-xylopyranosyl-(1->4)-α-L-rhamnopyranosyl-(1->2)-β-D-fucopyranosyl ester}-quillaic acid
   QA-TriR-FRXA—3-O-{α-L-rhamnopyranosyl-(1->3)-[3-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid
   QA-TriR-FRXX—3-O-{α-L-rhamnopyranosyl-(1->3)-[3-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid
   QA-TriX—3-O-{β-D-xylopyranosyl-(1->3)-[β-D-galactopyranosyl-(1->2)]-β-D-glucopyranosiduronic acid}-quillaic acid
   QA-TriX-F—3-O-{β-D-xylopyranosyl-(1->3)-[β-D-galactopyranosyl-(1->2)]-β-D-glucopyranosiduronic acid}-28-O-{β-D-fucopyranosyl ester}-quillaic acid
   QA-TriX-FR—3-O-{β-D-xylopyranosyl-(1->3)-[β-D-galactopyranosyl-(1->2)]-3-D-glucopyranosiduronic acid}-28-O-{α-L-rhamnopyranosyl-(1->2)-β-D-fucopyranosyl ester}-quillaic acid
   QA-TriX-FRX—3-O-{β-D-xylopyranosyl-(1->3)-[3-D-galactopyranosyl-(1->2)]-3-D-glucopyranosiduronic acid}-28-O-{β-D-xylopyranosyl-(1->4)-α-L-rhamnopyranosyl-(1->2)-β-D-fucopyranosyl ester}-quillaic acid
   QA-TriX-FRXA—3-O-{β-D-xylopyranosyl-(1->3)-[3-D-galactopyranosyl-(1->2)]-3-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid
   QA-TriX-FRXX—3-O-{β-D-xylopyranosyl-(1->3)-[3-D-galactopyranosyl-(1->2)]-3-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid
   QA-TriX-G—3-O-{β-D-xylopyranosyl-(1->3)-[3-D-galactopyranosyl-(1->2)]-3-D-glucopyranosiduronic acid}-28-O-{β-D-glucopyranosyl ester}-quillaic acid
   QA-TriX-GR—3-O-{β-D-xylopyranosyl-(1->3)-[β-D-galactopyranosyl-(1->2)]-3-D-glucopyranosiduronic acid}-28-O-{α-L-rhamnopyranosyl-(1->2)-β-D-glucopyranosyl ester}-quillaic acid
   QA-TriX-GRX—3-O-{β-D-xylopyranosyl-(1->3)-[3-D-galactopyranosyl-(1->2)]-3-D-glucopyranosiduronic acid}-28-O-{β-D-xylopyranosyl-(1->4)-α-L-rhamnopyranosyl-(1->2)-β-D-glucopyranosyl ester}-quillaic acid
   QA-Tri(X/R)—QA glycosylated at C-3 position with a branched trisaccharide which is either QA-TriX or QA-TriR
   QA-Tri(X/R)-F—QA glycosylated at C-28 and C-3 positions, which is either QA-TriX-F or QA-TriR-F
   QA-Tri(X/R)-FR—QA glycosylated at C-28 and C-3 positions, which is either QA-TriX-FR or QA-TriR-FR
   QA-Tri(X/R)-FRX—QA glycosylated at C-28 and C-3 positions, which is either QA-TriX-FRX or QA-TriR-FRX
   QA-Tri(X/R)-FRXA—QA glycosylated at C-28 and C-3 positions, which is either QA-TriX-FRXA or QA-TriR-FRXA
   QA-Tri(X/R)-FRXX—QA glycosylated at C-28 and C-3 positions, which is either QA-TriX-FRXX or QA-TriR-FRXX
   QA-Tri(X/R)-FRX(X/A)—QA glycosylated at C-28 and C-3 positions, which is either QA-TriX-FRXX, QA-TriX-FRXA, QA-TriR-FRXX or QA-TriR-FRXA
   QA-F—QA mono-glycosylated at the C-28 position.
   QA-FR—QA di-glycosylated at the C-28 position.
   QA-FRX—QA tri-glycosylated at the C-28 position.
   QA-FRXA—QA tetra-glycosylated at the C-28 position.
   QA-FRXX—QA tetra-glycosylated at the C-28 position.
   QA-FRX(X/A)—QA glycosylated at the C-28 position, which is either QA-FRXX or QA-FRXA.
Qs_0283850—Q. saponaria QA-Di α-1,3-L-rhamnosyltransferase
Qs_0283870—Q. saponaria QA-Di β-1,3-D-xylosyltransferase
Qs-28-O-ApiT4—Quillaic acid 28-O-fucoside [1,2]-rhamnoside [1,4] xyloside [1,3]
apiosyltransferase
Qs-28-O-FucT—Quillaic acid 28-O-fucosyltransferase
Qs-28-O-RhaT—Quillaic acid 28-O-fucoside [1,2]-rhamnosyltransferase
Qs-28-O-XylT3—Quillaic acid 28-O-fucoside [1,2]-rhamnoside [1,4] xylosyltransferase
Qs-28-O-XylT4—Quillaic acid 28-O-fucoside [1,2]-rhamnoside [1,4] xyloside [1,3] xylosyltransferase
Qs-3-O-GalT—Q. saponaria QA-Mono β-1,2-D-galactosyltransferase
Qs-3-O-RhaT—Q. saponaria QA-Di α-1,3-L-rhamnosyltransferase
Qs-3-O-RhaT/XylT—Q. saponaria QA-Di dual β-1,3-D-xylosyltransferase/α-1,3-L-rhamnosyltransferase
Qs-3-O-XylT—Q. saponaria QA-Di β-1,3-D-xylosyltransferase
QsAXS1—UDP-D-apiose/UDP-D-xylose synthase
QsbAS—Q. saponaria β-amyrin synthase
QsCSL1—Q. saponaria cellulose synthase-like enzyme (quillaic acid 3-O-glucuronosyltransferase)
QsCsIG2—Q. saponaria cellulose synthase-like enzyme (quillaic acid 3-O-glucuronosyltransferase)
QsCYP716-C-28—Q. saponaria quillaic acid C-28 oxidase
QsCYP716-C-16a—Q. saponaria quillaic acid C-16a oxidase
QsCYP714-C-23—Q. saponaria quillaic acid C-23 oxidase
QsFSL-1—Enzyme from Q. saponaria boosting the production of fucosylated saponins
QsFSL-2—Enzyme from Q. saponaria boosting the production of fucosylated saponins
QsFucSyn—Enzyme from Q. saponaria boosting the production of fucosylated saponins
QsUGT_A6—synonymous with Qs-28-O-RhaT
QsUGT_A7—synonymous with Qs-28-O-XylT3
QsUGT_D2—synonymous with Qs-28-O-ApiT4
QsUGT_D3—synonymous with Qs-28-O-XylT4
QsUGT_L2—synonymous with Qs-28-O-FucT
Rhap—L-Rhamnopyranose
RHM—Rhamnose synthase
SDR—Short chain dehydrogenase/reductase superfamily
SOAP3—Spinacia oleracea Medicagenic acid C-2P oxidase. Also known as CYP72A255.
SOAP4—Spinacia oleracea Medicagenic acid C-23 oxidase. Also known as CYP72A654.
SOAP5—Spinacia oleracea Medicagenic acid 3-O-glucuronosyltransferase
SOAP6—Spinacia oleracea Medicagenic acid-3-O-GIcA C-28 D-fucosyltransferase, also known as UGT74BB2
SoFSL-1—Enzyme from S. officinalis boosting the production of fucosylated saponins
SpolFSL—Spinacia oleracea FucSyn-like enzyme
tH MGR—Avena strigosa (diploid oat) truncated 3-hydroxy, 3-methylbutyryl-CoA reductase
UDP-sugar—Uridine diphosphate sugar
UGT—UDP-dependent glycosyltransferases
Xylp—D-Xylopyranose

### REFERENCES

Arias M A et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144.
Coler R N et al. (2011) Development and Characterization of Synthetic Glucopyranosyl Lipid Adjuvant System as a Vaccine Adjuvant. PLoS ONE 6(1): e16333. doi:10.1371/journal.pone.0016333
de Costa F, Barber C J S, Kim Y B, Reed D W, Zhang H, Fett-Neto A G, Covello P S. 2017. Molecular cloning of an ester-forming triterpenoid: UDP-glucose 28-O-glucosyltransferase involved in saponin biosynthesis from the medicinal plant Centella asiatica. Plant Sci 262: 9-17.
Errey J C, Mukhopadhyay B, Kartha K P, Field R A. 2004. Flexible enzymatic and chemo-enzymatic approaches to a broad range of uridine-diphospho-sugars. Chem Commun (Camb)(23): 2706-2707.
Faust T, Theurer C, Eger K, Kreis W. 1994. Synthesis of Uridine 5′-(α-D-Fucopyranosyl Diphosphate) and (Digitoxigenin-3β-yl)-β-D-Fucopyranoside and Enzymatic β-D-Fucosylation of Cardenolide Aglycones in Digitalis lanata1. Bioorganic Chemistry 22(2): 140-149.
Jozwiak, A., Sonawane, P., Panda, S., Garagounis, C., Papadopoulou, K. K., Abebie, B., Massalha, H., Almekias-Siegl, E., Scherg, T., Aharoni, A. 2020 Plant terpenoid metabolism co-opts a component of the cell wall biosynthesis machinery. Nat Chem Biol, 16(7): 740-748.
Kautsar S A, Suarez Duran H G, Blin K, Osbourn A, Medema M H. 2017. plantiSMASH: automated identification, annotation and expression analysis of plant biosynthetic gene clusters. Nucleic Acids Res
Kensil, C. R., Patel, U., Lennick, Marciani D. 1991, Separation and characterization of saponins with adjuvant activity from Quillaja saponaria Molina cortex, J. Immunol, 146 (2) 431-437
Kumar S, Stecher G, Tamura K. 2016. MEGA7: Molecular Evolutionary Genetics Analysis Version 7.0 for Bigger Datasets. Mol Biol Evol 33(7): 1870-1874.
Parakkottil Chothi M, Duncan G A, Armirotti A, Abergel C, Gurnon J R, Van Etten J L, Bernardi C, Damonte G, Tonetti M. 2010. Identification of an L-Rhamnose Synthetic Pathway in Two Nucleocytoplasmic Large DNA Viruses. Journal of Virology 84(17): 8829-8838.
Räbinä J, Mäki M, Savilahti E M, Järvinen N, Penttilä L, Renkonen R. 2001. Analysis of nucleotide sugars from cell lysates by ion-pair solid-phase extraction and reversed-phase high-performance liquid chromatography. Glycoconjugate Journal 18(10): 799-805.
Ragupathi G, Gardner J, Livingston P, Gin D 2011 Natural and Synthesis saponin adjuvant QS-21 for vaccines against cancer. Expert Rev. Vaccines, 10(4) 463-470.
Reed J, Stephenson M J, Miettinen K, Brouwer B, Leveau A, Brett P, Goss R J M, Goossens A, O'Connell M A, Osbourn A. 2017. A translational synthetic biology platform for rapid access to gram-scale quantities of novel drug-like molecules. Metab Eng 42: 185-193.
Rejzek M, Hill L, Hems E S, Kuhaudomlarp S, Wagstaff B A, Field R A. 2017. Profiling of Sugar Nucleotides. Methods Enzymol 597: 209-238
Ross, J., Li, Y., Lim, E. K., Bowles, D. J., 2001. Higher plant glycosyltransferases. Genome Biol. 2, 1-6. https://doi.org/10.1186/gb-2001-2-2-reviews3004
Sainsbury F, Thuenemann E C, Lomonossoff G P. 2009. pEAQ: versatile expression vectors for easy and quick transient expression of heterologous proteins in plants. Plant Biotechnol J 7(7): 682-693.
Saitou N, Nei M. 1987. The neighbor-joining method: a new method for reconstructing phylogenetic trees. Mol Biol Evol 4(4): 406-425.
Sasaki N, Nishizaki Y, Ozeki Y, Miyahara T. 2014. The role of acyl-glucose in anthocyanin modifications. Molecules 19(11): 18747-18766.
Stephenson M. J. et al., 2018, Transient Expression in Nicotiana Benthamiana Leaves for Triterpene Production at a Preparative Scale. Journal of visualized experiments: JoVE, (138), p. 58169.
Wang P., et al., 2005, Synthesis of the potent immunostimulatory adjuvant QS-21A., J A M Chem Soc, 127(10) pp 3256-3257

1) A method of making QA-FRX(X/A), wherein the FRX(X/A) chain is added at the C-28 position of QA, the method comprising:
(i) (a) combining QA with UDP-α-D-fucose and the enzyme Qs-28-O-FucT (SEQ ID NO 2) or an enzyme with a sequence with at least 70% sequence identity to form QA-F; and/or
(b) combining QA with UDP-4-keto, 6-deoxy-D-glucose, the enzyme Qs-28-O-FucT (SEQ ID NO 2) or an enzyme with a sequence with at least 70% sequence identity, and the enzyme QsFucSyn (SEQ ID NO 12) or an enzyme with a sequence with at least 45% sequence identity to form QA-F;
(ii) combining QA-F with UDP-β-L-rhamnose and the enzyme Qs-28-O-RhaT (SEQ ID NO 4) or an enzyme with a sequence with at least 70% sequence identity to form QA-FR;
(iii) combining QA-FR with UDP-α-D-xylose and the enzyme Qs-28-O-XylT3 (SEQ ID NO 6) or an enzyme with a sequence with at least 70% sequence identity to form QA-FRX; and
(iv) combining QA-FRX with UDP-α-D-xylose and the enzyme Qs-28-O-XylT4 (SEQ ID NO 8) or an enzyme with a sequence with at least 70% sequence identity to form QA-FRXX, and/or combining QA-FRX with UDP-α-D-apiose and the enzyme Qs-28-O-ApiT4 (SEQ ID NO 10) or an enzyme with a sequence with at least 70% sequence identity to form QA-FRXA.

2) A method of making QA-Mono-FRX(X/A), QA-Di-FRX(X/A) and/or QA-Tri(X/R)-FRX(X/A), wherein the Mono, Di or Tri(X/R) chain is added at the C-3 position and the FRX(X/A) chain is added at the C-28 position of QA, the method comprising:
(i) combining QA with UDP-α-D-glucuronic acid and the enzyme QsCSL1 (SEQ ID NO 26) or QsCsIG2 (SEQ ID NO 28) or an enzyme with a sequence with at least 70% sequence identity to form QA-Mono; optionally
(ii) combining QA-Mono with UDP-α-D-galactose and the enzyme Qs-3-O-GalT (SEQ ID NO 30) or an enzyme with a sequence with at least 70% sequence identity to form QA-Di; optionally
(iii) combining QA-Di with UDP-β-L-rhamnopyranose and the enzyme DN20529_c0_g2_i8 (SEQ ID NO 36) or Qs_0283850 (SEQ ID NO 34), or Qs-3-O-RhaT/XylT (SEQ ID NO 32) or an enzyme with a sequence with at least 70% sequence identity to form QA-TriR, and/or combining QA-Di with UDP-α-D-xylopyranose and the enzyme Qs_0283870 (SEQ ID NO 38) or Qs-3-O-RhaT/XylT (SEQ ID NO 32) or an enzyme with a sequence with at least 70% sequence identity to form QA-TriX;
(iv) (a) combining QA-Mono, QA-Di and/or QA-Tri(R/X) with UDP-α-D-fucose and the enzyme Qs-28-O-FucT (SEQ ID NO 2) or an enzyme with a sequence with at least 70% sequence identity to form QA-Mono-F, QA-Di-F and/or QA-Tri(R/X)-F, and/or
(b) combining QA-Mono, QA-Di and/or QA-Tri(R/X) with UDP-4-keto, 6-deoxy-D-glucose, the enzyme Qs-28-O-FucT (SEQ ID NO 2) or an enzyme with a sequence with at least 70% sequence identity, and the enzyme QsFucSyn (SEQ ID NO 12) or an enzyme with a sequence with at least 45% sequence identity to form QA-Mono-F, QA-Di-F and/or QA-Tri(R/X)-F;
(v) combining QA-Mono-F, QA-Di-F and/or QA-Tri(R/X)-F with UDP-β-L-rhamnose and the enzyme Qs-28-O-RhaT (SEQ ID NO 4) or an enzyme with a sequence with at least 70% sequence identity to form QA-Mono-FR, QA-Di-FR and/or QA-Tri(R/X)-FR;
(vi) combining QA-Mono-FR, QA-Di-FR and/or QA-Tri(R/X)-FR with UDP-α-D-xylose and the enzyme Qs-28-O-XylT3 (SEQ ID NO 6) or an enzyme with a sequence with at least 70% sequence identity to form QA-Mono-FRX, QA-Di-FRX and/or QA-Tri(R/X)-FRX; and
(vii) combining QA-Mono-FRX, QA-Di-FRX and/or QA-Tri(R/X)-FRX with UDP-α-D-xylose and the enzyme Qs-28-O-XylT4 (SEQ ID NO 8) or an enzyme with a sequence with at least 70% sequence identity to form QA-Mono-FRXX, QA-Di-FRXX and/or QA-Tri(R/X)-FRXX, and/or combining QA-Mono-FRX, QA-Di-FRX and/or QA-Tri(R/X)-FRX with UDP-α-D-apiose and the enzyme Qs-28-O-ApiT4 (SEQ ID NO 10) or an enzyme with a sequence with at least 70% sequence identity to form QA-Mono-FRXA, QA-Di-FRXA and/or QA-Tri(R/X)-FRXA.

3) A method of making a biosynthetic 3-O-{α-L-rhamnopyranosyl-(1->3)-[3-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid (QA-TriR-FRXA) in a host, which method comprises the steps of:
a) expressing genes required for the biosynthesis of QA-TriR, and
b) introducing a nucleic acid encoding:
i. the enzyme Qs-28-O-FucT (SEQ ID NO 2) or an enzyme with a sequence with at least 70% sequence identity to SEQ ID NO 2;
ii. the enzyme Qs-28-O-RhaT (SEQ ID NO 4) or an enzyme with a sequence with at least 70% sequence identity to SEQ ID NO 4;
iii. the enzyme Qs-28-O-XylT3 (SEQ ID NO 6) or an enzyme with a sequence with at least 70% sequence identity to SEQ ID NO 6; and
iv. the enzyme Qs-28-O-ApiT4 (SEQ ID NO 10) or an enzyme with a sequence with at least 70% sequence identity to SEQ ID NO 10;
into the host.

4) A method of making a biosynthetic 3-O-{α-L-rhamnopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriR-FRXX) in a host, which method comprises the steps of:
a) expressing genes required for the biosynthesis of QA-TriR, and
b) introducing a nucleic acid encoding:
i. the enzyme Qs-28-O-FucT (SEQ ID NO 2) or an enzyme with a sequence with at least 70% sequence identity to SEQ ID NO 2;
ii. the enzyme Qs-28-O-RhaT (SEQ ID NO 4) or an enzyme with a sequence with at least 70% sequence identity to SEQ ID NO 4;
iii. the enzyme Qs-28-O-XylT3 (SEQ ID NO 6) or an enzyme with a sequence with at least 70% sequence identity to SEQ ID NO 6; and
iv. the enzyme Qs-28-O-XylT4 (SEQ ID NO 8) or an enzyme with a sequence with at least 70% sequence identity to SEQ ID NO 8;
into the host.

5) A method of making a biosynthetic 3-O-{β-D-xylopyranosyl-(1->3)-[β-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid (QA-TriX-FRXA) in a host, which method comprises the steps of:
a) expressing genes required for the biosynthesis of QA-TriX, and
b) introducing a nucleic acid encoding:
i. the enzyme Qs-28-O-FucT (SEQ ID NO 2) or an enzyme with a sequence with at least 70% sequence identity to SEQ ID NO 2;
ii. the enzyme Qs-28-O-RhaT (SEQ ID NO 4) or an enzyme with a sequence with at least 70% sequence identity to SEQ ID NO 4;
iii. the enzyme Qs-28-O-XylT3 (SEQ ID NO 6) or an enzyme with a sequence with at least 70% sequence identity to SEQ ID NO 6; and
iv. the enzyme Qs-28-O-ApiT4 (SEQ ID NO 10) or an enzyme with a sequence with at least 70% sequence identity to SEQ ID NO 10;
into the host.

6) A method of making a biosynthetic 3-O-{β-D-xylopyranosyl-(1->3)-[β-D-galactopyranosyl-(1->2)]-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid (QA-TriX-FRXX) in a host, which method comprises the steps of:
a) expressing genes required for the biosynthesis of QA-TriX, and
b) introducing a nucleic acid encoding:
i. the enzyme Qs-28-O-FucT (SEQ ID NO 2) or an enzyme with a sequence with at least 70% sequence identity to SEQ ID NO 2;
ii. the enzyme Qs-28-O-RhaT (SEQ ID NO 4) or an enzyme with a sequence with at least 70% sequence identity to SEQ ID NO 4;
iii. the enzyme Qs-28-O-XylT3 (SEQ ID NO 6) or an enzyme with a sequence with at least 70% sequence identity to SEQ ID NO 6; and
iv. the enzyme Qs-28-O-XylT4 (SEQ ID NO 8) or an enzyme with a sequence with at least 70% sequence identity to SEQ ID NO 8;
into the host.

7) A method of making a biosynthetic QA-Tri(X/R)-FRX(X/A)) in a host, which method comprises the steps of:
a) expressing genes required for the biosynthesis of QA-TriX and/or QA-TriR, and
b) introducing a nucleic acid encoding:
i. the enzyme Qs-28-O-FucT (SEQ ID NO 2) or an enzyme with a sequence with at least 70% sequence identity to SEQ ID NO 2;
ii. the enzyme Qs-28-O-RhaT (SEQ ID NO 4) or an enzyme with a sequence with at least 70% sequence identity to SEQ ID NO 4;
iii. the enzyme Qs-28-O-XylT3 (SEQ ID NO 6) or an enzyme with a sequence with at least 70% sequence identity to SEQ ID NO 6; and
iv. the enzyme Qs-28-O-XylT4 (SEQ ID NO 8) or an enzyme with a sequence with at least 70% sequence identity to SEQ ID NO 8 and/or the enzyme Qs-28-O-ApiT4 (SEQ ID NO 10) or an enzyme with a sequence with at least 70% sequence identity to SEQ ID NO 10;
into the host.

8) The method of claim 3, claim 4 or claim 7, wherein the biosynthesis of QA-TriR in step a) is obtained by introducing a nucleic acid encoding (i) (a) the enzyme QsCSL1 (SEQ ID NO 26) or an enzyme with a sequence with at least 70% sequence identity to SEQ ID NO 26, or (b) the enzyme QsCsIG2 (SEQ ID NO 28) or an enzyme with a sequence with at least 70% sequence identity to SEQ ID NO 28; (ii) the enzyme Qs-3-O-GalT (SEQ ID NO 30) or an enzyme with a sequence with at least 70% sequence identity to SEQ ID NO 30 and (iii) (a) the enzyme DN20529_c0_g2_i8 (SEQ ID NO 36) or an enzyme with a sequence with at least 70% sequence identity to SEQ ID NO 36, or (b) the enzyme Qs_0283850 (SEQ ID NO 34) or an enzyme with a sequence with at least 70% sequence identity to SEQ ID NO 34, or (c) the enzyme Qs-3-O-RhaT/XylT (SEQ ID NO 32) or an enzyme with a sequence with at least 70% sequence identity to SEQ ID NO 32.

9) The method of claim 5, claim 6 or claim 7, wherein the biosynthesis of QA-TriX in step a) is obtained by introducing a nucleic acid encoding (i) (a) the enzyme QsCSL1 (SEQ ID NO 26) or an enzyme with a sequence with at least 70% sequence identity to SEQ ID NO 26, or (b) the enzyme QsCsIG2 (SEQ ID NO 28) or an enzyme with a sequence with at least 70% sequence identity to SEQ ID NO 28; (ii) the enzyme Qs-3-O-GalT (SEQ ID NO 30) or an enzyme with a sequence with at least 70% sequence identity to SEQ ID NO 30, and (iii) (a) the enzyme Qs_0283870 (SEQ ID NO 38) or an enzyme with a sequence with at least 70% sequence identity to SEQ ID NO 38, or (b) the enzyme Qs-3-O-RhaT/XylT (SEQ ID NO 32) or an enzyme with a sequence with at least 70% sequence identity to SEQ ID NO 32.

10) The method of any one of claims 3 to 9, wherein
amino acid SEQ ID NO 2 is encoded by nucleic acid SEQ ID NO 1;
amino acid SEQ ID NO 4 is encoded by nucleic acid SEQ ID NO 3;
amino acid SEQ ID NO 6 is encoded by nucleic acid SEQ ID NO 5;
amino acid SEQ ID NO 8 is encoded by nucleic acid SEQ ID NO 7;
amino acid SEQ ID NO 10 is encoded by nucleic acid SEQ ID NO 9.

11) The method of any one of claims 2, 8, 9 or 10, wherein:
amino acid SEQ ID NO 26 is encoded by nucleic acid SEQ ID NO 25;
amino acid SEQ ID NO 28 is encoded by nucleic acid SEQ ID NO 27;
amino acid SEQ ID NO 30 is encoded by nucleic acid SEQ ID NO 29;
amino acid SEQ ID NO 32 is encoded by nucleic acid SEQ ID NO 31;
amino acid SEQ ID NO 34 is encoded by nucleic acid SEQ ID NO 33;
amino acid SEQ ID NO 36 is encoded by nucleic acid SEQ ID NO 35;
amino acid SEQ ID NO 38 is encoded by nucleic acid SEQ ID NO 37.

12) A fucosyltransferase enzyme according to SEQ ID NO 2 (Qs-28-O-FucT) or an enzyme with a sequence with at least 70% sequence identity.

13) A rhamnosyltransferase enzyme according to SEQ ID NO 4 (Qs-28-O-RhaT) or an enzyme with a sequence with at least 70% sequence identity.

14) A xylosyltransferase enzyme according to SEQ ID NO 6 (Qs-28-O-XylT3) or an enzyme with a sequence with at least 70% sequence identity.

15) A xylosyltransferase enzyme according to SEQ ID NO 8 (Qs-28-O-XylT4) or an enzyme with a sequence with at least 70% sequence identity.

16) An apiosyltransferase enzyme according to SEQ ID NO 10 (Qs-28-O-ApiT4) or an enzyme with a sequence with at least 70% sequence identity.

17) A nucleic acid which encodes any of the enzymes as claimed in any one of claims 12 to 16.

18) A nucleic acid according to claim 17, further encoding any enzyme as claimed in claim 8 or 9.

19) A vector comprising the nucleic acid according to claim 17 or claim 18.

20) A host cell comprising the nucleic acid according to claim 17 or claim 18.

21) A host cell transformed with the vector according to claim 19.

22) A host cell according to claim 20 or 21, wherein the host cell is a plant cell or a microbial cell.

23) A biological system of a plant or a microorganism comprising host cells according to claim 21 or claim 22.

24) A biological system according to claim 23, wherein the biological system is yeast or Nicotiana benthamiana.

25) An oxidoreductase enzyme according to SEQ ID NO 12 (QsFucSyn) or an enzyme with a sequence with at least 45% sequence identity.

26) The oxidoreductase enzyme according to claim 25, wherein the enzyme with at least 45% sequence identity to SEQ ID NO 12 (QsFucSyn) is one of QsFSL-1 (SEQ ID No. 48), QsFSL-2 (SEQ ID No 50) or SoFSL-1 (SEQ ID No 52).

27) A nucleic acid which encodes the enzymes according to claim 25 or claim 26.

28) A method according to any one of claim 1 or claim 2, with step (i)(b) (claim 1) or step (iv)(b) (claim 2), wherein the enzyme with at least 45% sequence identity to SEQ ID NO 12 (QsFucSyn) is according to claim 26 or is SpolFSL (SEQ ID NO 54).

29) A method according to any one of claims 3 to 11, wherein the step b) further introduces a nucleic acid encoding at least one of the enzymes according to claim 25 or claim 26, or encoding the enzyme SpolFSL (SEQ ID NO 54).

30) A method according to any one of claim 1 or claim 2, with step (i)(b) (claim 1) or step (iv)(b) (claim 2), wherein UDP-D-glucose is combined with the enzyme ATCV-1 (SEQ ID NO 40) or an enzyme with a sequence with at least 55% sequence identity to form said UDP-4-keto, 6-deoxy-D-glucose,

31) The method according to claim 29, wherein the step b) further introduces a nucleic acid encoding the enzyme ATCV-1 (SEQ ID NO 40) or an enzyme with a sequence with at least 55% sequence identity.

32) A UDP-apiose/UDP-xylose synthase enzyme according to SEQ ID NO 14 (QsAXS1) or an enzyme with a sequence with at least 70% sequence identity.

33) A nucleic acid encoding the enzyme according to claim 32.

34) A method according to claim 1 or claim 2, with step (i)(b) (claim 1) or step (iv)(b) (claim 2), wherein the step (i)(b) (claim 1) or step (iv)(b) (claim 2) further includes combining with the enzyme according to claim 32.

35) A method according to any one of claims 1 to 11, 28 to 31, and 34, wherein the method further includes the step of isolating the QA derivative.

36) The QA derivative obtained according to the method of claim 35.

37) The use of the QA derivative according to claim 36 as an adjuvant

38) The use according to claim 37, wherein the adjuvant is a liposomal formulation.

39) The use according to claim 37 or claim 38, wherein the adjuvant further comprises a TLR4 agonist.

40) The use according to claim 39, wherein the TLR4 agonist is 3D-MPL.

41) An adjuvant composition comprising the QA derivative according to claim 36.
